(19) United States
(12) Patent Application Publication
(10) Pub. No.: US 2009/0227602 A1
(43) Pub. Date: Sep. 10, 2009

(54) COMPOSITIONS AND TREATMENTS FOR INHIBITING KINASE AND/OR HMG-COA REDUCTASE

(76) Inventors: John Griffin, Atherton, CA (US); Guido Lanza, San Francisco, CA (US); Jessen Yu, Mountain View, CA (US)

Correspondence Address: WILSON SONSINI GOODRICH & ROSATI, 650 PAGE MILL ROAD, PALO ALTO, CA, 94304-1050, US

(21) Appl. No.: 12/090,434

(22) PCT Filed: Oct. 30, 2006
(86) PCT No.: PCT/US06/42558

§ 371 Date: Mar. 13, 2009

Related U.S. Application Data
(60) Provisional application No. 60/731,422, filed on Oct. 28, 2005.

(51) Int. Cl. A61K 31/4439; C07D 401/14; A61P 37/06

(52) U.S. Cl. 514/256; 544/333; 546/274.1; 514/341

(57) ABSTRACT

The present invention provides compositions of matter, kits and methods for their use in the treatment of MAP kinase-related conditions and/or HMG-CoA reductase-related conditions. In particular, the invention provides compositions for treating inflammatory and/or cardiovascular conditions in an animal subject by inhibiting p38αMAP kinase and/or HMG-CoA reductase, as well as providing formulations and modes of administering such compositions. The invention further provides methods for the rational design of inhibitors of MAP kinase, HMG-CoA reductase, or both for use in the practice of the present invention.

### RELATED APPLICATIONS

This application claims priority to U.S. Provisional Patent Applications Ser. No. 60/731,422, filed Oct. 28, 2005, such application being hereby incorporated by reference in its entirety, for all purposes.

### BACKGROUND

The pro-inflammatory cytokines, such as tumor necrosis factor-α (TNF-α) and interleukin-1β (IL-1β), contribute to the pathogenesis of various allergic, inflammatory and autoimmune diseases. Consequently, multiple therapeutic approaches have been aimed at reducing the expression and/or activity of such pro-inflammatory cytokines. Examples of these include the use of IL-1 receptor antagonists, TNF-α converting enzyme inhibitors, and inhibitors of certain enzymes that play a role in signal transduction pathways associated with inflammation, including responses to and expression of TNF-α and IL-1β.

Immunomodulatory and inflammatory effects also play a role in cardiovascular conditions, such as atherogenesis and its associated cardiovascular risks, such as atherosclerosis, thrombosis, myocardial infarction, ischemic stroke, ischemic-reperfusion injury and peripheral vascular diseases. For example, inflammatory responses, including those involving TNF-α and IL-1β, play a role in the initiation, growth and disruption of atheroslerotic plaques. Treatments of such cardiovascular conditions typically address hypercholesterolemia, for example, by inhibiting the enzymes involved in cholesterol biosynthesis. Statins, for example, inhibit 3-hydroxy-3-methylglutaryl CoA (HMG-CoA) reductase, the rate-limiting enzyme in the cholesterol biosynthesis pathway.

With heart disease being the most prevalent illness of industrialized counties, and inflammatory conditions affecting millions of individuals worldwide, there remains a need for compounds that can treat one or both of these types of conditions. These compounds can form the basis for pharmaceutical compositions useful in the prevention and treatment of atherogenesis and/or inflammatory conditions in humans and other mammals. Moreover, the interplay between inflammatory and cardiovascular conditions means that compounds or combinations of compounds addressing both may be particularly beneficial.

### BRIEF SUMMARY OF THE INVENTION

In one aspect, the present invention provides compounds and compositions that show MAP kinase inhibitory activity and/or HMG-CoA reductase inhibitory activity. Some embodiments are compounds comprising novel analogs of MAP kinase inhibitors. Some embodiments are compounds comprising novel analogs of HMG-CoA reductase inhibitors. Some embodiments are compounds comprising novel series of substituted imidazoles, substituted pyrazoles, or substituted pyrroles. Some embodiments are compounds comprising novel series of substituted indoles, substituted pyridines, substituted pyrimidines, substituted quinolines, pyranopyridines, pyridazines, pyrrolopyridines, naphthyridines, benzenes, ethylenes, or isoquinolines. Some embodiments are compounds comprising structures modified to favor and/or enforce a closed ring structure, e.g, a δ-lactam or a des-oxo-structure. Some embodiments are combinations comprising two more compounds described herein and/or two or more forms of a compound described herein.

In another aspect, the present invention provides methods of treating a MAP kinase- and/or an HMG-CoA reductase-related condition by administering an effective amount of a compound or combination of compounds to a subject. In some embodiments, known inhibitors of HMG-CoA reductase are used to inhibit a MAP kinase, e.g., p38αMAP kinase, in the treatment of a MAP kinase-related condition or in the treatment of both a MAP kinase- and an HMG-CoA reductase-related condition. In other embodiments, novel compounds that inhibit both a MAP kinase and HMG-CoA reductase are superior to compounds that target a MAP kinase but not HMG-CoA reductase or to compounds that target HMG-CoA reductase and not MAP kinase, for example, in treating a MAP kinase- and/or an HMG-CoA reductase-related condition. In some embodiments, novel combinations of compounds or forms of compounds are used to treat MAP kinase- and/or HMG-CoA reductase-related conditions that are inflammatory conditions. For example, in some embodiments, combinations comprising a statin lactone and a salt form of a hydroxy acid statin are used to treat skin and/or vascular inflammatory conditions. In preferred embodiments, such combinations provide synergistic effects in treating inflammation.

In another aspect, the present invention provides pharmaceutical compositions, formulations and modes of administering one or more compounds, e.g., compounds of the present invention, for use in methods of treating a MAP kinase-related and/or an HMG-CoA reductase-related condition, including inflammatory conditions. For example, in some embodiments, a statin lactone can be formulated with a hydroxy acid form of the same or different statin, a pharmaceutically acceptable salt thereof, or with another active agent. For example, in some embodiments, a statin lactone can be formulated with a non-statin anti-inflammatory agent. Such combination formulations are administered orally or topically in preferred embodiments, e.g., in the treatment of inflammatory conditions.

In one aspect, the present invention provides compositions that show MAP kinase inhibitory and/or 3-hydroxy-3-methyl glutaryl-coenzyme A reductase (HMG-CoA reductase) inhibitory activity. Some embodiments are compositions comprising novel analogs of MAP kinase inhibitors. Some embodiments are compositions comprising novel analogs of HMG-CoA reductase inhibitors. Some embodiments are compositions comprising structures modified to favor and/or enforce a closed ring structure, e.g., a δ-lactam or a des-oxo-structure.

In another aspect, the present invention provides methods of treating an inflammatory condition by administering an effective amount of a pharmaceutical composition, e.g., a composition of the present invention, to a subject. In other embodiments, the invention provides methods which target both a MAP kinase, e.g., p38αMAP kinase and HMG-CoA reductase for inhibition that are superior to methods that that target a MAP kinase but not HMG-CoA reductase or to methods that target HMG-CoA reductase and not MAP kinase, for example, in the treatment of a MAP kinase- and/or an HMG-CoA reductase-related condition.

In another aspect, the present invention relates to the use, in the treatment of a MAP kinase-related conditions that are inflammatory diseases and disorders associated with inflammation, of pharmaceutical compositions comprising combinations of a lactone form of a “statin” inhibitor of the enzyme 3-hydroxy-3-methyl glutaryl-coenzyme A reductase (HMG-CoA reductase) with one or more additional pharmacologically active agents. Some embodiments are the use of a pharmaceutical composition comprising a combination of a statin lactone and a hydroxy acid statin salt, e.g., as a therapy to treat inflammatory diseases and disorders associated with inflammation, preferably vascular diseases and disorders. Some embodiments are the use of a pharmaceutical composition comprising a combination of a statin lactone and a hydroxy acid statin salt, e.g., as a topical therapy to treat inflammatory diseases and disorders of the skin. Some embodiments are the use of a pharmaceutical composition comprising a combination of a statin lactone and a non-statin anti-inflammatory agent, e.g., as a therapy to treat inflammatory diseases and disorders. In some embodiments, a combination of a statin lactone and another active agent provides a synergistic effect in treating a MAP kinase-related condition, e.g., an inflammatory condition.

One aspect of the present invention provides compositions and methods of inhibiting a MAP kinase comprising administering an effective amount of at least one compound comprising formula XX:

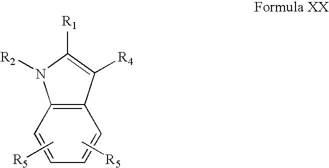

wherein R₁ is

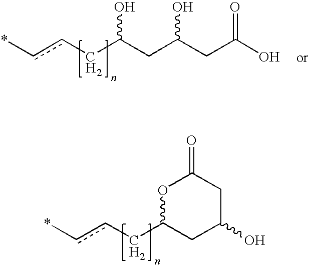

n being 0 or any integer;

R₂ is optionally substituted alkyl, cycloalkyl, aryl, or heteroaryl;

R₄ is optionally substituted

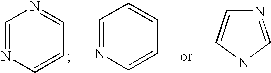

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula XX:

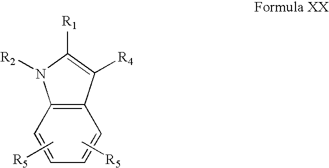

wherein R₁ is

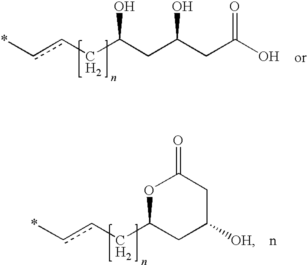

being 0 or any integer;

R₂ is optionally substituted alkyl, cycloalkyl, aryl, or heteroaryl;

R₄ is optionally substituted

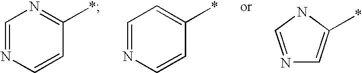

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides compositions and methods of inhibiting a MAP kinase comprising administering an effective amount of at least one compound comprising formula XXI:

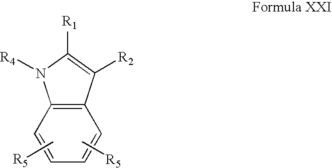

wherein R₁ is

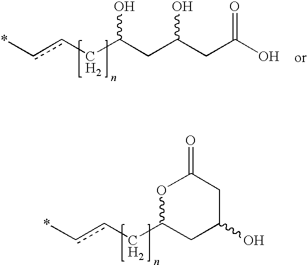

n being 0 or any integer;

R₂ is optionally substituted alkyl, cycloalkyl, aryl, or heteroaryl;

R₄ is optionally substituted

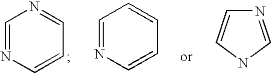

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula XXI:

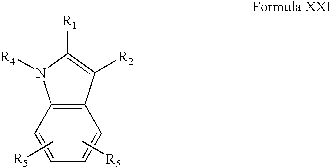

wherein R₁ is

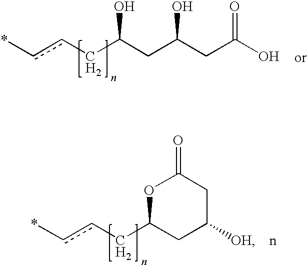

being 0 or any integer;

R₂ is optionally substituted alkyl, cycloalkyl, aryl, or heteroaryl;

R₄ is optionally substituted

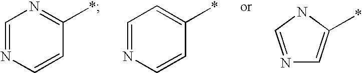

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides compositions and methods of inhibiting a MAP kinase comprising administering an effective amount of at least one compound comprising formula XXII:

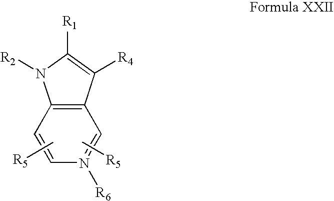

wherein R₁ is

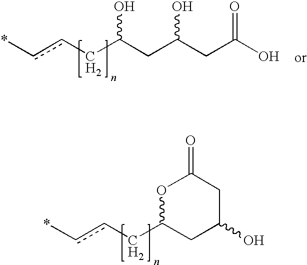

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present/invention provides a compound comprising formula XXII:

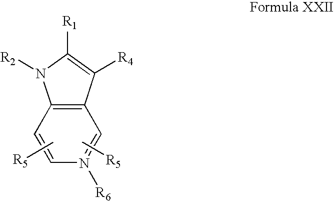

wherein R₁ is

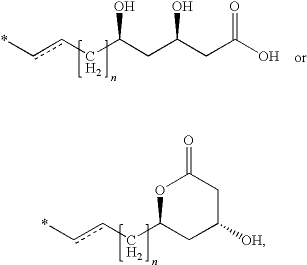

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXIII:

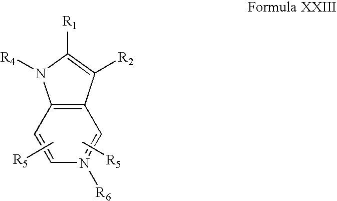

wherein R₁ is

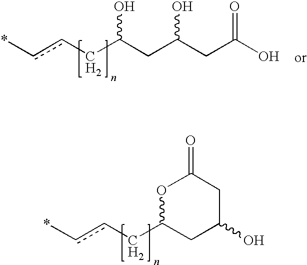

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXIII:

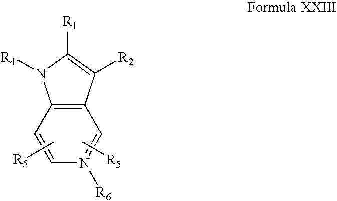

wherein R₁ is

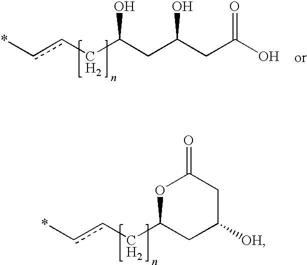

n being 0 or any integer;

R₂ is optionally substituted alkyl, cycloalkyl, aryl, or heteroaryl;

R₄ is optionally substituted alkyl, cycloalkyl, aryl, or heteroaryl

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXIV:

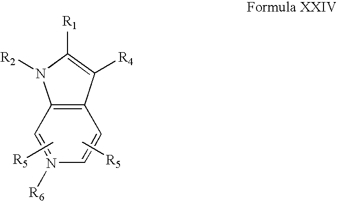

wherein R₁ is

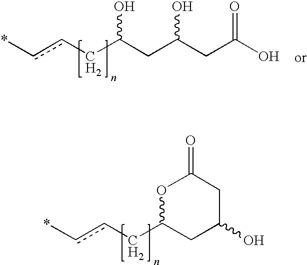

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXIV:

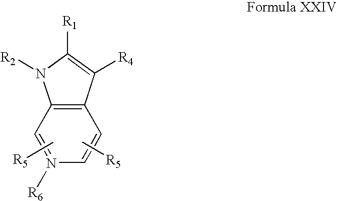

wherein R₁ is

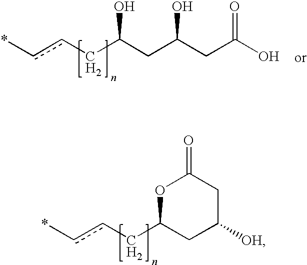

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXV:

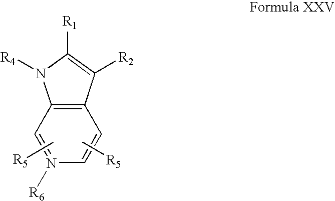

wherein R₁ is

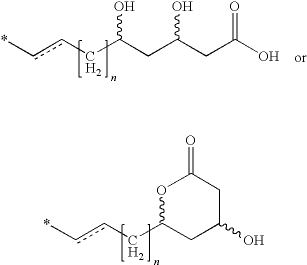

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXV:

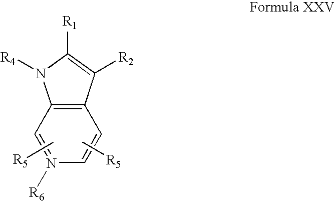

wherein R₁ is

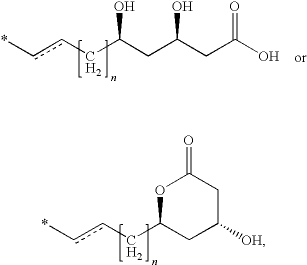

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXVI:

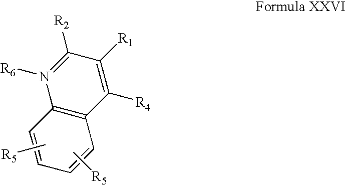

wherein R₁ is

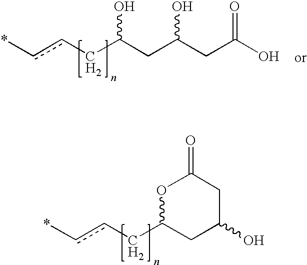

n being 0 or any integer;

R₂ is optionally substituted alkyl, cycloalkyl, aryl, or heteroaryl;

R₄ is optionally substituted

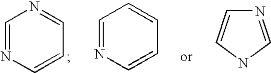

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

Another aspect of the present invention provides a compound comprising formula XXVI:

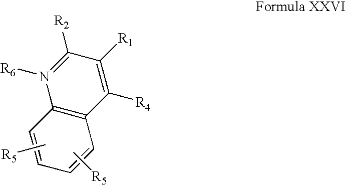

wherein R₁ is

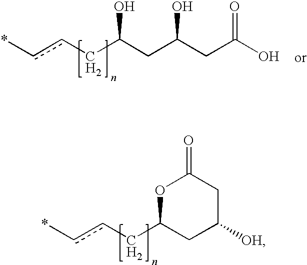

n being 0 or any integer;

R₂ is optionally substituted alkyl, cycloalkyl, aryl, or heteroaryl;

R₄ is optionally substituted

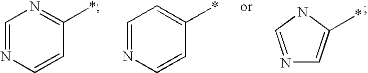

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

One aspect of the present invention provides compounds and methods of inhibiting a MAP kinase comprising administering an effective amount of at least one compound comprising formula XXVII:

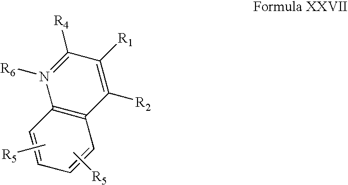

wherein R₁ is

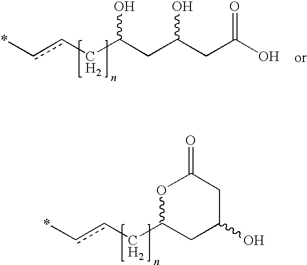

n being 0 or any integer;

R₂ is optionally substituted alkyl, cycloalkyl, aryl, or heteroaryl;

R₄ is optionally substituted

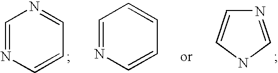

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

Another aspect of the present invention provides a compound comprising formula XXVII:

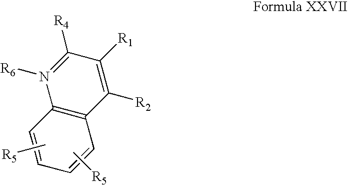

wherein R₁ is

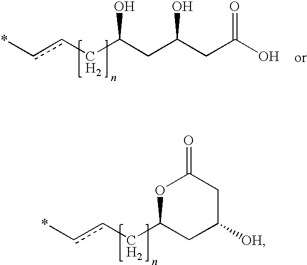

n being 0 or any integer;

R₂ is optionally substituted alkyl, cycloalkyl, aryl, or heteroaryl;

R₄ is optionally substituted

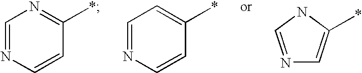

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

One aspect of the present invention provides compounds and methods of inhibiting a MAP kinase comprising administering an effective amount of at least one compound comprising formula XXVIII:

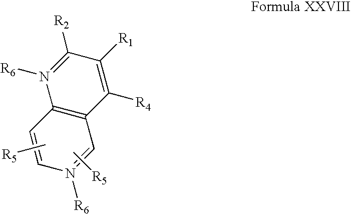

wherein R₁ is

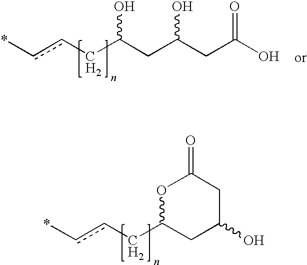

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXVIII:

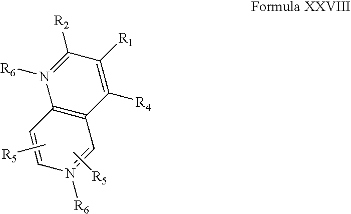

wherein R₁ is

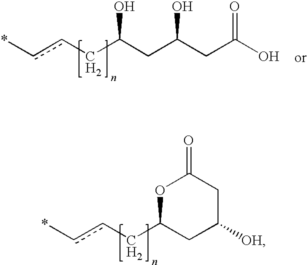

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, optionally substituted amino; and

R₆ is either a lone pair of electrons or an oxygen atom.

One aspect of the present invention provides compounds and methods of inhibiting a MAP kinase comprising administering an effective amount of at least one compound comprising formula XXIX:

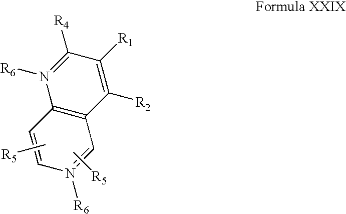

wherein R₁ is

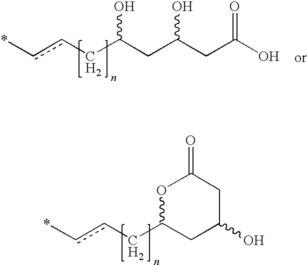

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXIX:

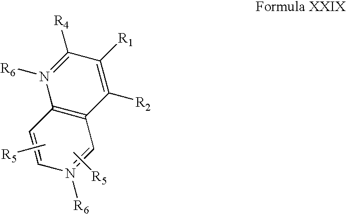

wherein R₁ is

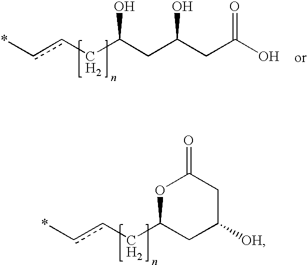

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXX:

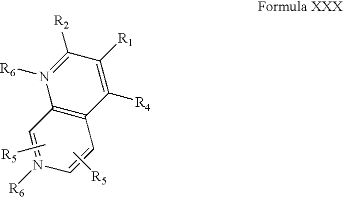

wherein R₁ is

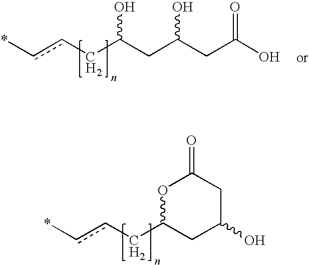

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXX:

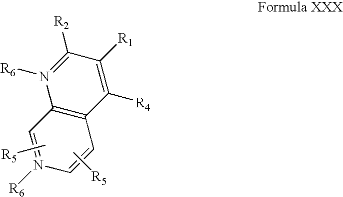

wherein R₁ is

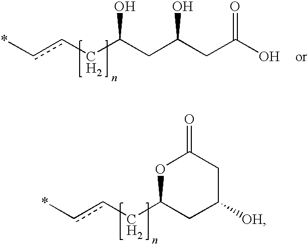

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXXI:

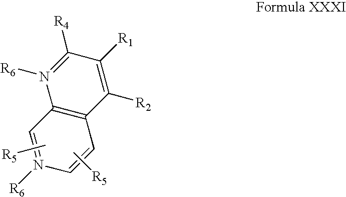

wherein R₁ is

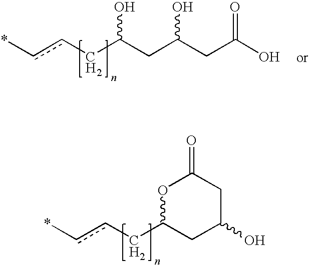

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXXI:

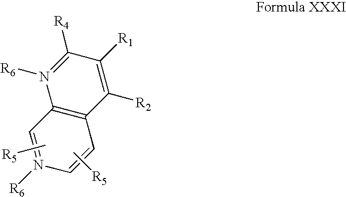

wherein R₁ is

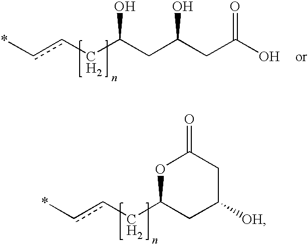

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Some embodiments provide a compound comprising formula XXXII:

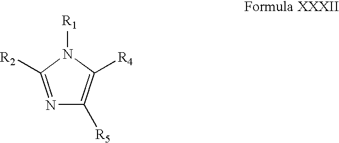

wherein R₁ is

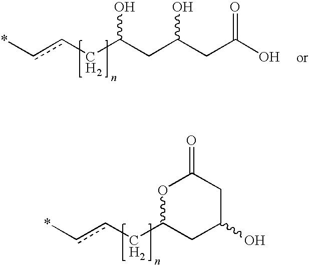

n being 0 or any integer;

R₂ is optionally substituted alkyl, cycloalkyl, aryl, or heteroaryl;

R₄ is optionally substituted

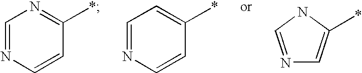

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXII:

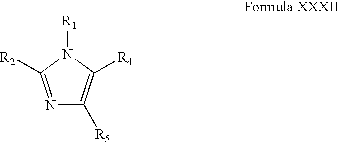

wherein R₁ is

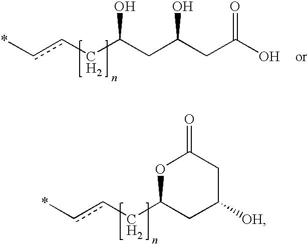

n being 0 or any integer;

R₂ is optionally substituted alkyl, cycloalkyl, aryl, or heteroaryl;

R₄ is optionally substituted

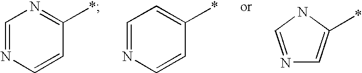

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXIII:

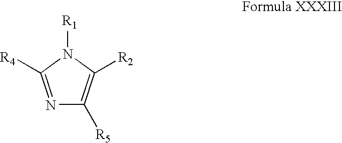

wherein R₁ is

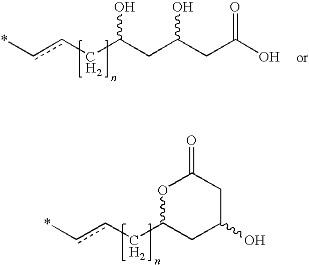

n being 0 or any integer;

R₂ is optionally substituted alkyl, cycloalkyl, aryl, or heteroaryl;

R₄ is optionally substituted

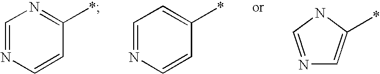

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXIII:

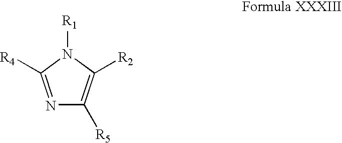

wherein R₁ is

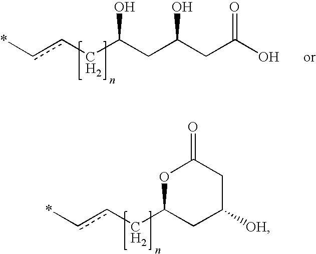

n being 0 or any integer;

R₂ is optionally substituted alkyl, cycloalkyl, aryl, or heteroaryl;

R₄ is optionally substituted

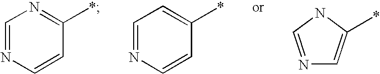

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXIV:

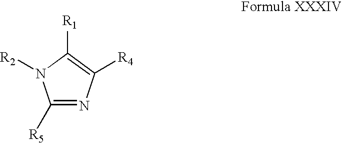

wherein R₁ is

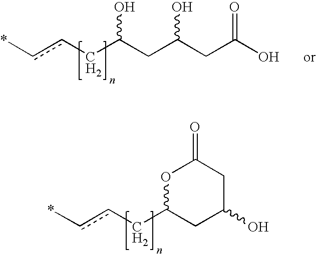

n being 0 or any integer;

R₂ is optionally substituted alkyl, cycloalkyl, aryl, or heteroaryl;

R₄ is optionally substituted

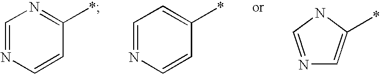

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXIV:

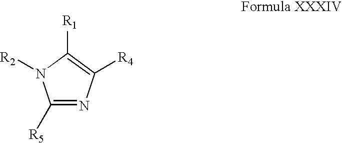

wherein R₁ is

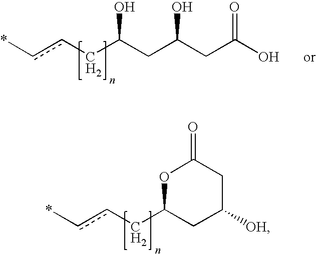

n being 0 or any integer;

R₂ is optionally substituted alkyl, cycloalkyl, aryl, or heteroaryl;

R₄ is optionally substituted

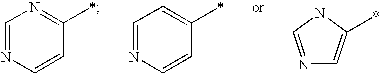

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXV:

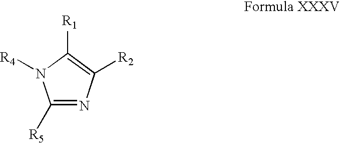

wherein R₁ is

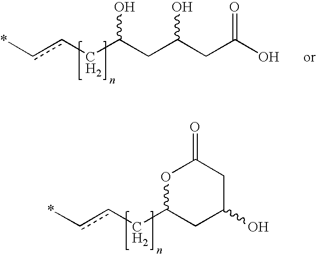

n being 0 or any integer;

R₂ is optionally substituted alkyl, cycloalkyl, aryl, or heteroaryl;

R₄ is optionally substituted

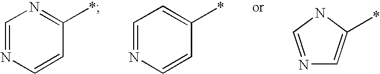

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXV:

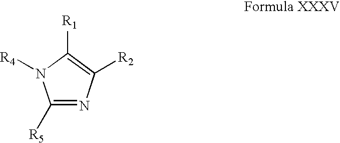

wherein R₁ is

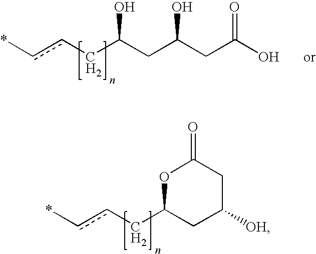

n being 0 or any integer;

R₂ is optionally substituted alkyl, cycloalkyl, aryl, or heteroaryl;

R₄ is optionally substituted

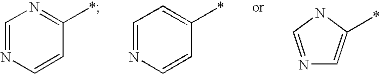

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXVI:

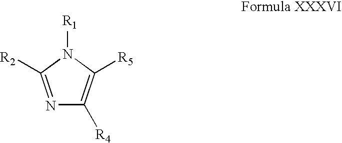

wherein R₁ is

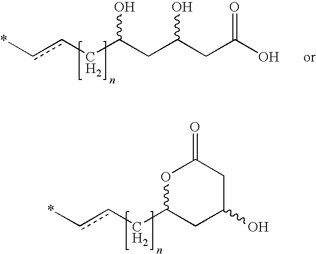

n being 0 or any integer;

R₂ is optionally substituted alkyl, cycloalkyl, aryl, or heteroaryl;

R₄ is optionally substituted

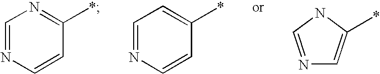

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXVI:

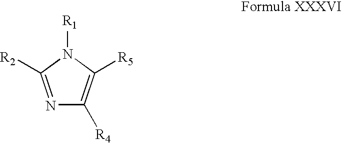

wherein R₁ is

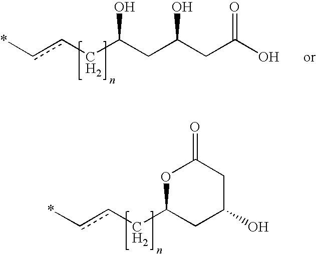

n being 0 or any integer;

R₂ is optionally substituted alkyl, cycloalkyl, aryl, or heteroaryl;

R₄ is optionally substituted

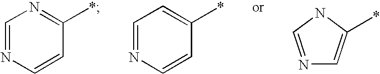

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXVII:

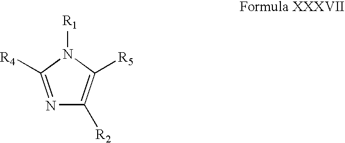

wherein R₁ is

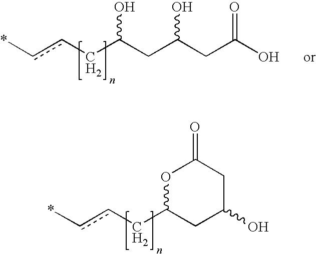

n being 0 or any integer;

R₂ is optionally substituted alkyl, cycloalkyl, aryl, or heteroaryl;

R₄ is optionally substituted

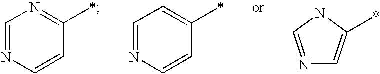

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXVII:

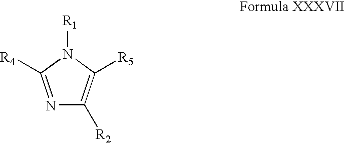

wherein R₁ is

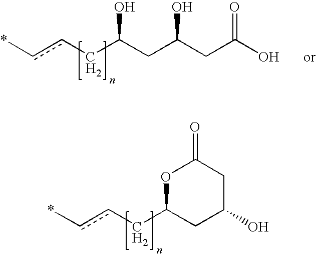

n being 0 or any integer;

R₂ is optionally substituted alkyl, cycloalkyl, aryl, or heteroaryl;

R₄ is optionally substituted

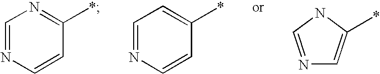

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXVIII:

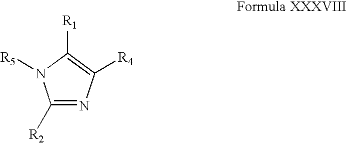

wherein R₁ is

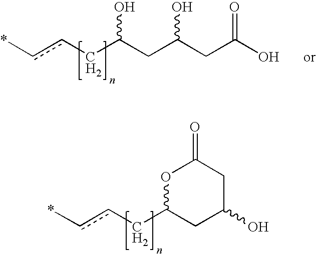

n being 0 or any integer;

R₂ is optionally substituted alkyl, cycloalkyl, aryl, or heteroaryl;

R₄ is optionally substituted

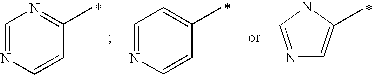

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXVIII:

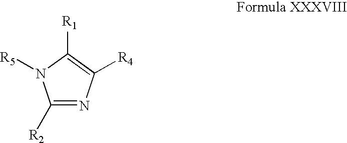

wherein R₁ is

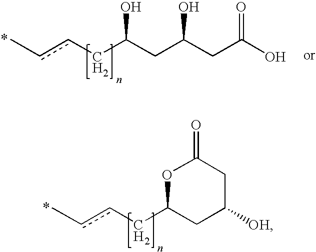

n being 0 or any integer;

R₂ is optionally substituted alkyl, cycloalkyl, aryl, or heteroaryl;

R₄ is optionally substituted

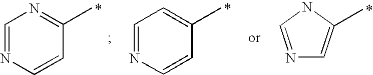

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXIX:

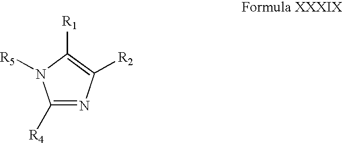

wherein R₁ is

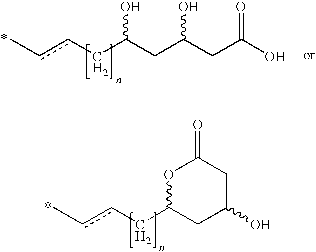

n being 0 or any integer;

R₂ is optionally substituted alkyl, cycloalkyl, aryl, or heteroaryl;

R₄ is optionally substituted

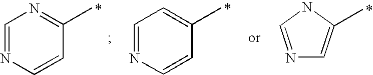

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXIX:

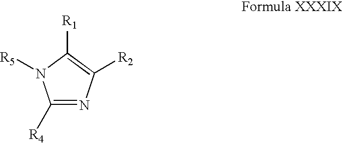

wherein R₁ is

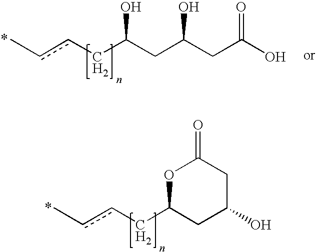

n being 0 or any integer;

R₂ is optionally substituted alkyl, cycloalkyl, aryl, or heteroaryl;

R₄ is optionally substituted

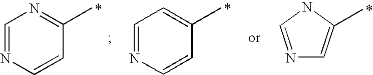

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXX:

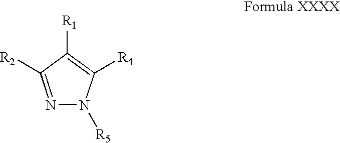

wherein R₁ is

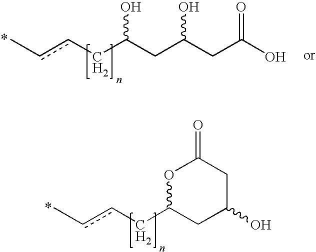

n being 0 or any integer;

R₂ is optionally substituted alkyl, cycloalkyl, aryl, or heteroaryl;

R₄ is optionally substituted

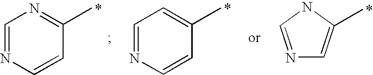

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXX

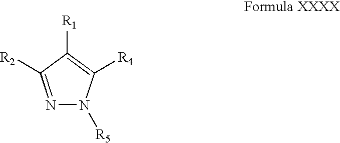

wherein R₁ is

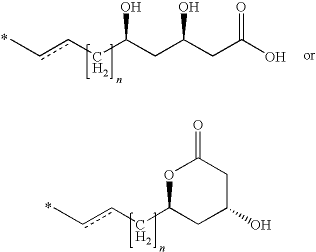

n being 0 or any integer;

R₂ is optionally substituted alkyl, cycloalkyl, aryl, or heteroaryl;

R₄ is optionally substituted

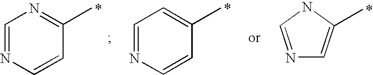

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXI:

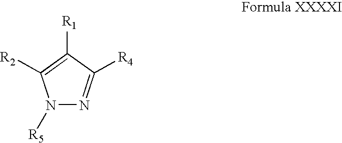

wherein R₁ is

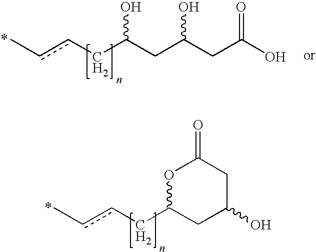

n being 0 or any integer;

R₂ is optionally substituted alkyl, cycloalkyl, aryl, or heteroaryl;

R₄ is optionally substituted

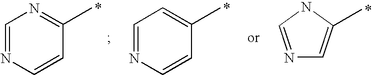

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXI:

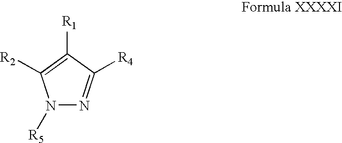

wherein R₁ is

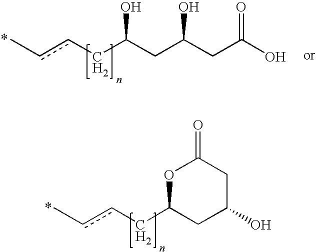

n being 0 or any integer;

R₂ is optionally substituted alkyl, cycloalkyl, aryl, or heteroaryl;

R₄ is optionally substituted

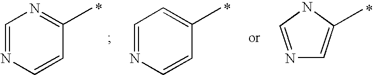

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXII:

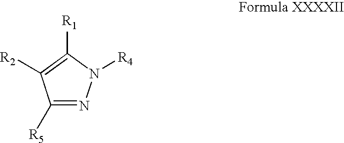

wherein R₁ is

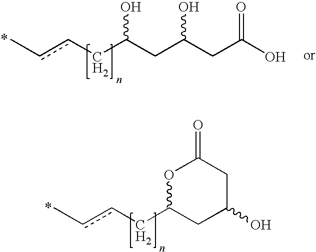

n being 0 or any integer;

R₂ is optionally substituted alkyl, cycloalkyl, aryl, or heteroaryl;

R₄ is optionally substituted

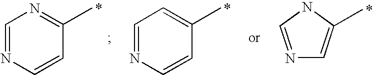

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXII:

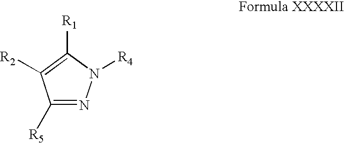

wherein R₁ is

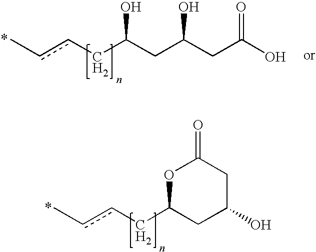

n being 0 or any integer;

R₂ is optionally substituted alkyl, cycloalkyl, aryl, or heteroaryl;

R₁ is optionally substituted

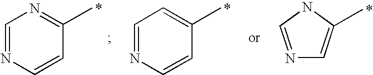

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXIII:

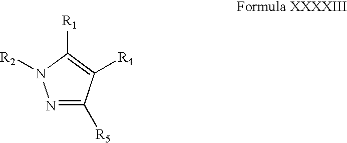

wherein R₁ is

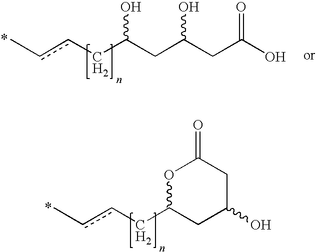

n being 0 or any integer;

R₂ is optionally substituted alkyl, cycloalkyl, aryl, or heteroaryl;

R₄ is optionally substituted

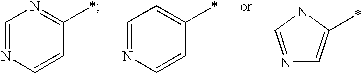

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXIII:

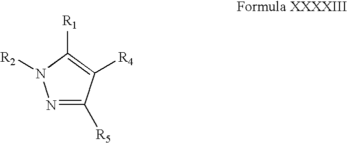

wherein R₁ is

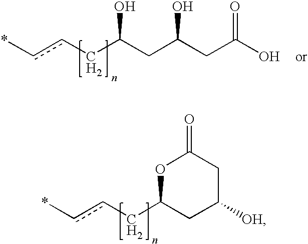

n being 0 or any integer;

R₂ is optionally substituted alkyl, cycloalkyl, aryl, or heteroaryl;

R₄ is optionally substituted

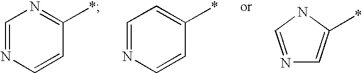

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXIV:

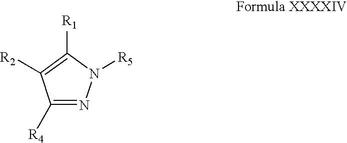

wherein R₁ is

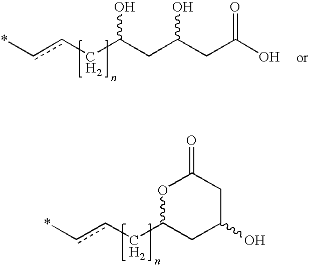

n being 0 or any integer;

R₂ is optionally substituted alkyl, cycloalkyl, aryl, or heteroaryl;

R₄ is optionally substituted

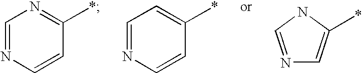

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXIV:

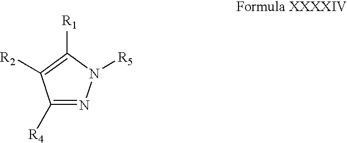

wherein R₁ is

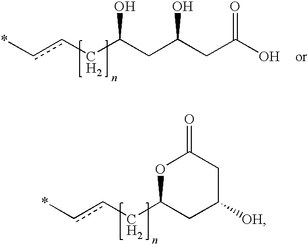

n being 0 or any integer;

R₂ is optionally substituted alkyl, cycloalkyl, aryl, or heteroaryl;

R₄ is optionally substituted

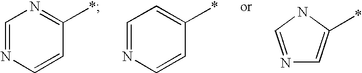

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXV:

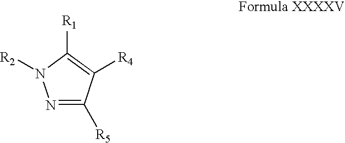

wherein R₁ is

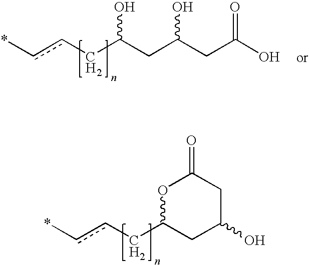

n being 0 or any integer;

R₂ is optionally substituted alkyl, cycloalkyl, aryl, or heteroaryl;

R₄ is optionally substituted

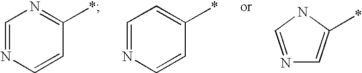

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXV:

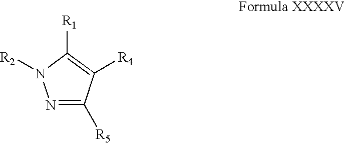

wherein R₁ is

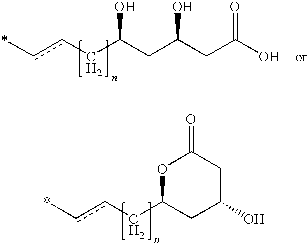

n being 0 or any integer;

R₂ is optionally substituted alkyl, cycloalkyl, aryl, or heteroaryl;

R₄ is optionally substituted

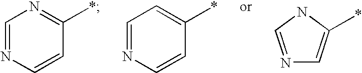

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXVI:

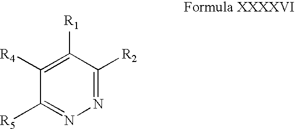

wherein R₁ is

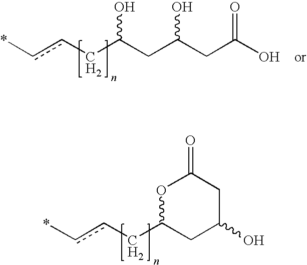

n being 0 or any integer;

R₂ is optionally substituted alkyl, cycloalkyl, aryl, or heteroaryl;

R₄ is optionally substituted

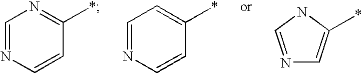

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXVI:

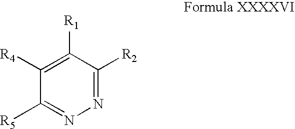

wherein R₁ is

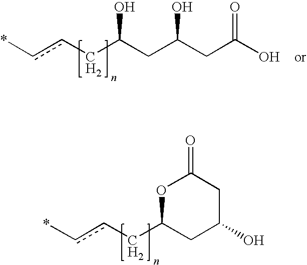

n being 0 or any integer;

R₂ is optionally substituted alkyl, cycloalkyl, aryl, or heteroaryl;

R₄ is optionally substituted

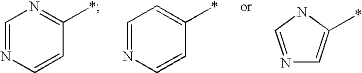

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXVII:

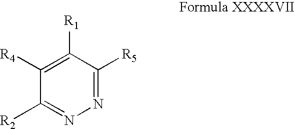

wherein R₁ is

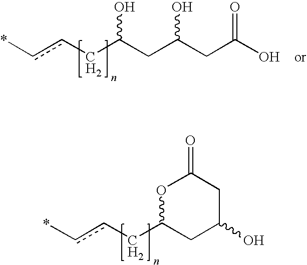

n being 0 or any integer;

R₂ is optionally substituted alkyl, cycloalkyl, aryl, or heteroaryl;

R₄ is optionally substituted

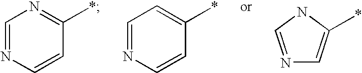

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXVII:

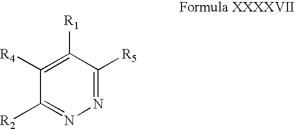

wherein R₁ is

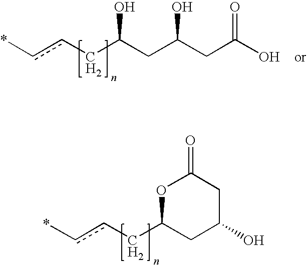

n being 0 or any integer;

R₂ is optionally substituted alkyl, cycloalkyl, aryl, or heteroaryl;

R₄ is optionally substituted

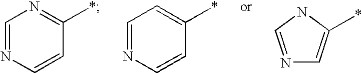

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXVIII:

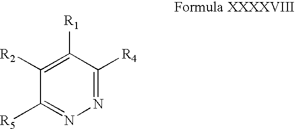

wherein R₁ is

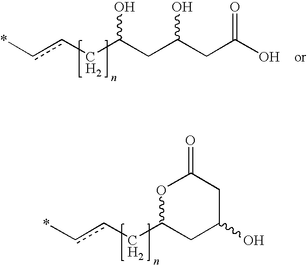

n being 0 or any integer;

R₂ is optionally substituted alkyl, cycloalkyl, aryl, or heteroaryl;

R₄ is optionally substituted

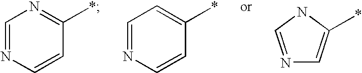

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXVIII:

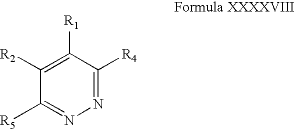

wherein R₁ is

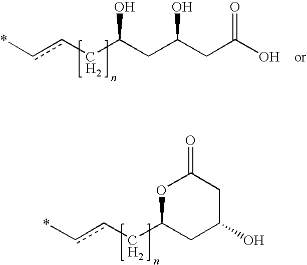

n being 0 or any integer;

R₂ is optionally substituted alkyl, cycloalkyl, aryl, or heteroaryl;

R₄ is optionally substituted

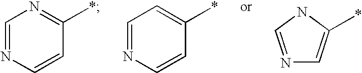

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXIX:

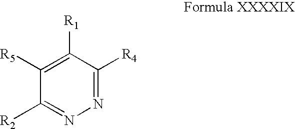

wherein R₁ is

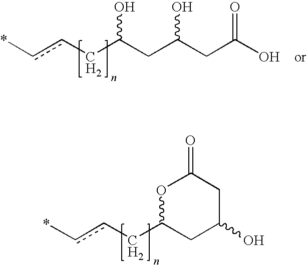

n being 0 or any integer;

R₂ is optionally substituted alkyl, cycloalkyl, aryl, or heteroaryl;

R₄ is optionally substituted

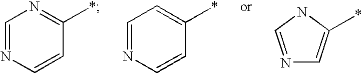

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXIX.

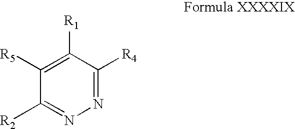

wherein R₁ is

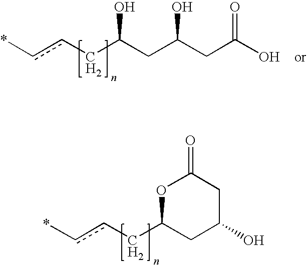

n being 0 or any integer;

R₂ is optionally substituted alkyl, cycloalkyl, aryl, or heteroaryl;

R₄ is optionally substituted

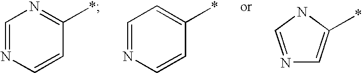

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula L:

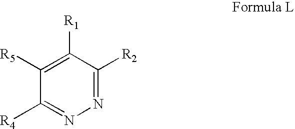

wherein R₁ is

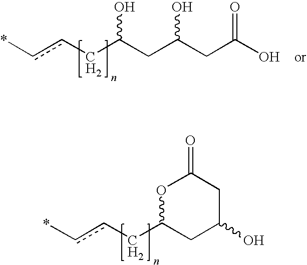

n being 0 or any integer;

R₂ is optionally substituted alkyl, cycloalkyl, aryl, or heteroaryl;

R₄ is optionally substituted

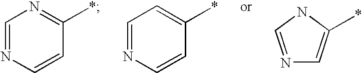

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula L:

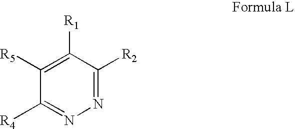

wherein R₁ is

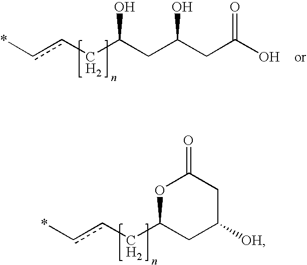

n being 0 or any integer;

R₂ is optionally substituted alkyl, cycloalkyl, aryl, or heteroaryl;

R₄ is optionally substituted

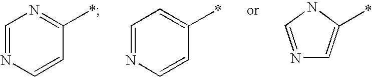

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LI:

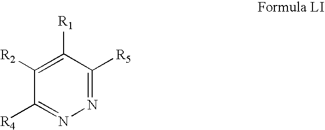

wherein R₁ is

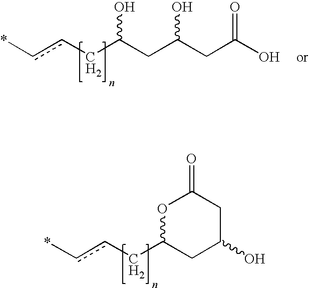

n being 0 or any integer;

R₂ is optionally substituted alkyl, cycloalkyl, aryl, or heteroaryl;

R₄ is optionally substituted

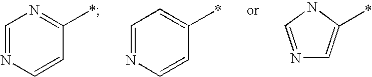

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LI:

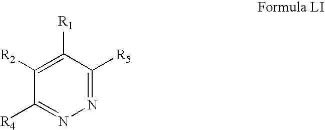

wherein R₁ is

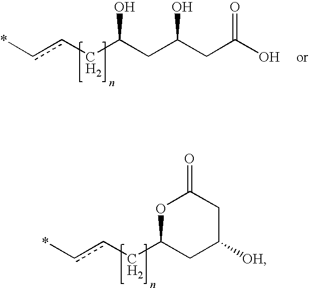

n being 0 or any integer;

R₂ is optionally substituted alkyl, cycloalkyl, aryl, or heteroaryl;

R₄ is optionally substituted

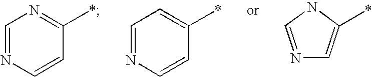

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LII:

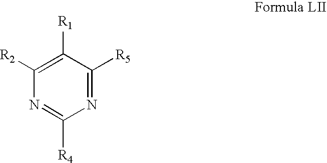

wherein R₁ is

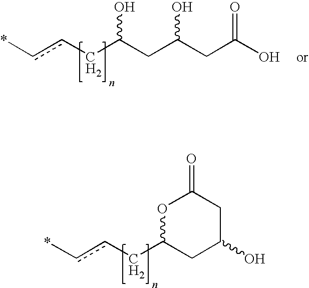

n being 0 or any integer;

R₂ is optionally substituted alkyl, cycloalkyl, aryl, or heteroaryl;

R₄ is optionally substituted

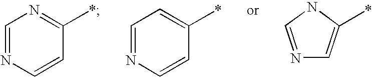

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LII:

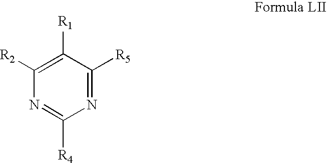

wherein R₁ is

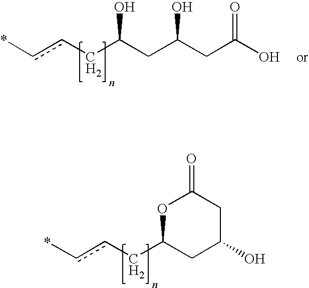

n being 0 or any integer;

R₂ is optionally substituted alkyl, cycloalkyl, aryl, or heteroaryl;

R₄ is optionally substituted

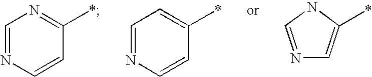

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LIII:

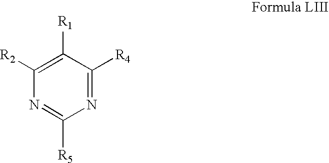

wherein R₁ is

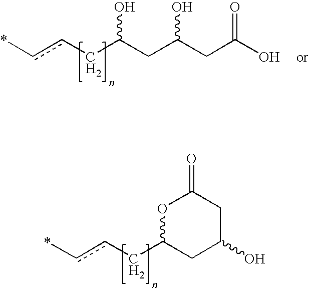

n being 0 or any integer;

R₂ is optionally substituted alkyl, cycloalkyl, aryl, or heteroaryl;

R₄ is optionally substituted

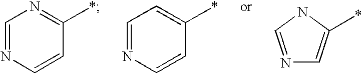

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LIII:

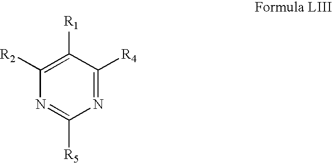

wherein R₁ is

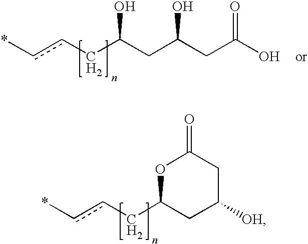

n being 0 or any integer;

R₂ is optionally substituted alkyl, cycloalkyl, aryl, or heteroaryl;

R₄ is optionally substituted

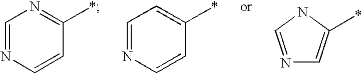

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LIV:

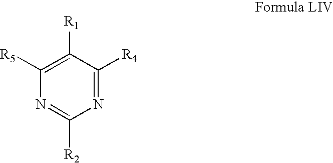

wherein R₁ is

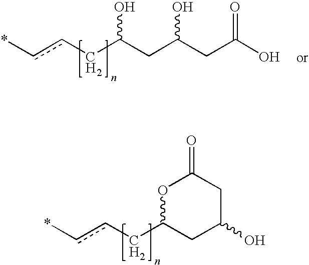

n being 0 or any integer;

R₂ is optionally substituted alkyl, cycloalkyl, aryl, or heteroaryl;

R₄ is optionally substituted

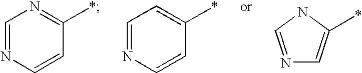

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LIV:

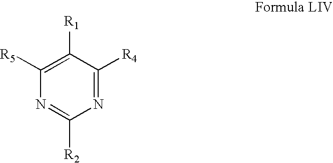

wherein R₁ is

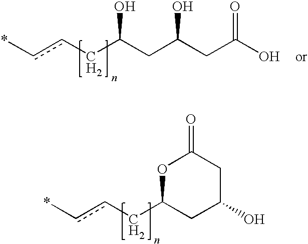

n being 0 or any integer;

R₂ is optionally substituted alkyl, cycloalkyl, aryl, or heteroaryl;

R₄ is optionally substituted

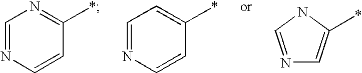

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

One aspect of the present invention provides methods of inhibiting a MAP kinase comprising administering an effective amount of at least one compound comprising formula LV:

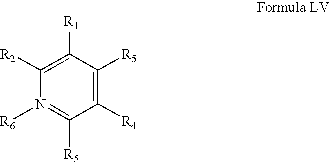

wherein R₁ is

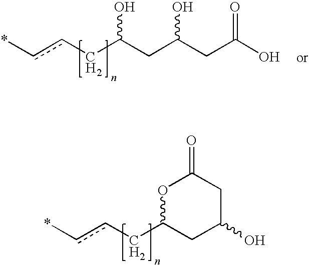

n being 0 or any integer;

R₂ is optionally substituted alkyl, cycloalkyl, aryl, or heteroaryl;

R₄ is optionally substituted

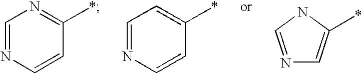

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LV:

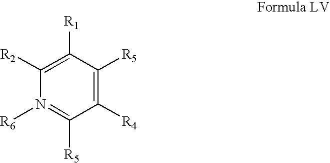

wherein R₁ is

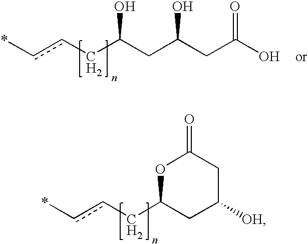

n being 0 or any integer;

R₂ is optionally substituted alkyl, cycloalkyl, aryl, or heteroaryl;

R₄ is optionally substituted

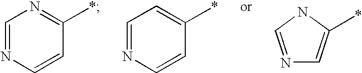

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LVI:

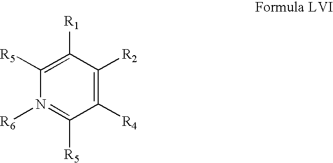

wherein R₁ is

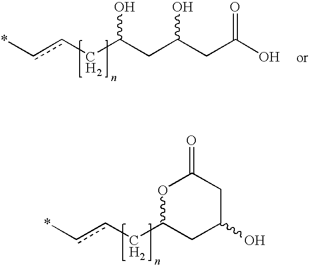

n being 0 or any integer;

R₂ is optionally substituted alkyl, cycloalkyl, aryl, or heteroaryl;

R₄ is optionally substituted

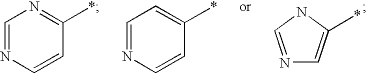

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LVI:

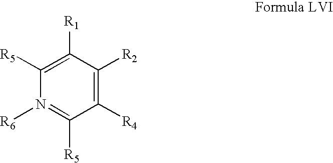

wherein R₁ is

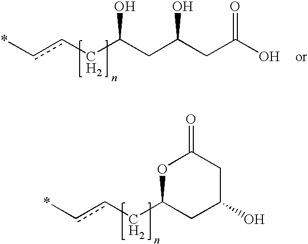

n being 0 or any integer;

R₂ is optionally substituted alkyl, cycloalkyl, aryl, or heteroaryl;

R₄ is optionally substituted

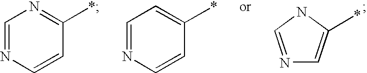

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LVII:

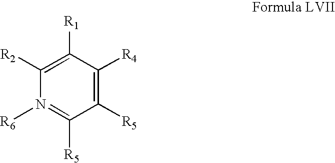

wherein R₁ is

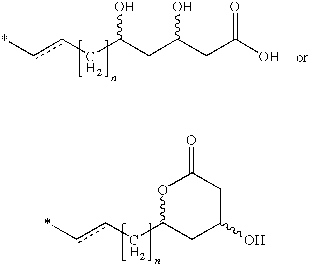

n being 0 or any integer;

R₂ is optionally substituted alkyl, cycloalkyl, aryl, or heteroaryl;

R₄ is optionally substituted

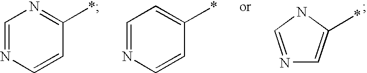

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LVII:

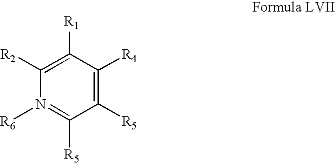

wherein R₁ is

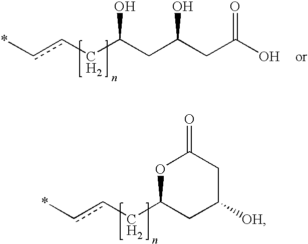

n being 0 or any integer;

R₂ is optionally substituted alkyl, cycloalkyl, aryl, or heteroaryl;

R₄ is optionally substituted

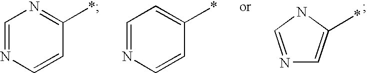

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LVIII:

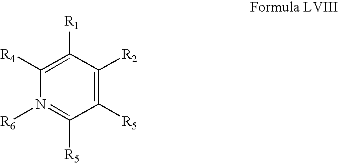

wherein R₁ is

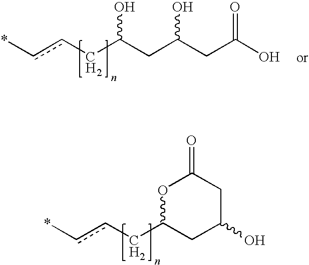

n being 0 or any integer;

R₂ is optionally substituted alkyl, cycloalkyl, aryl, or heteroaryl;

R₄ is optionally substituted

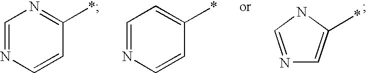

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LVIII:

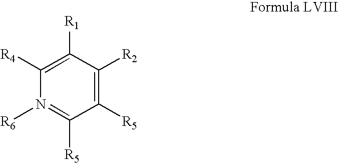

wherein R₁ is

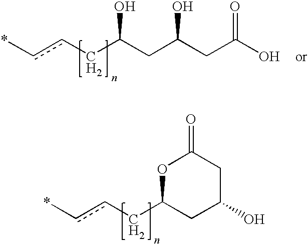

n being 0 or any integer;

R₂ is optionally substituted alkyl, cycloalkyl, aryl, or heteroaryl;

R₄ is optionally substituted

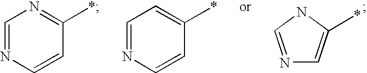

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LIX:

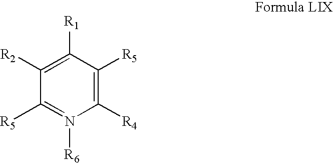

wherein R₁ is

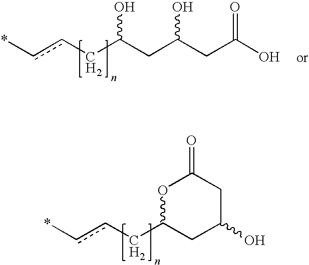

n being 0 or any integer;

R₂ is optionally substituted alkyl, cycloalkyl, aryl, or heteroaryl;

R₄ is optionally substituted

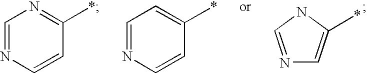

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LIX:

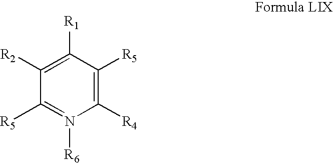

wherein R₁ is

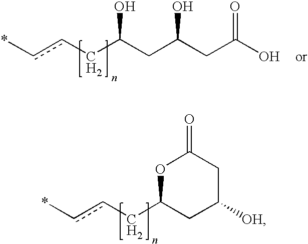

n being 0 or any integer;

R₂ is optionally substituted alkyl, cycloalkyl, aryl, or heteroaryl;

R₄ is optionally substituted

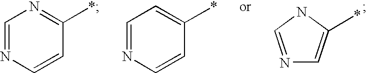

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LX:

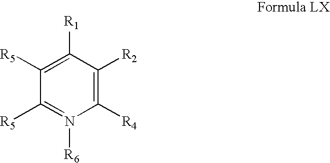

wherein R₁ is

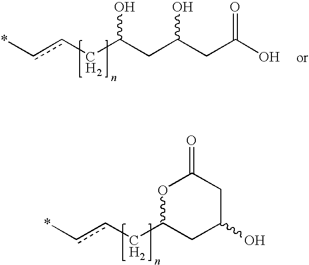

n being 0 or any integer;

R₂ is optionally substituted alkyl, cycloalkyl, aryl, or heteroaryl;

R₄ is optionally substituted

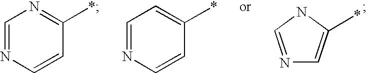

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LX:

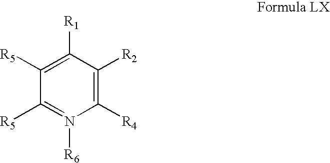

wherein R₁ is

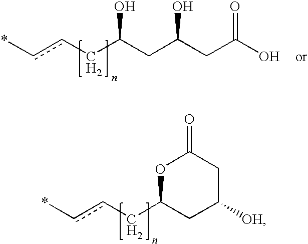

n being 0 or any integer;

R₂ is optionally substituted alkyl, cycloalkyl, aryl, or heteroaryl;

R₄ is optionally substituted

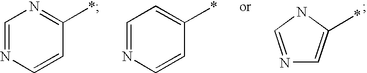

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXI:

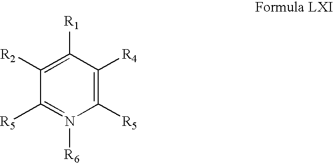

wherein R₁ is

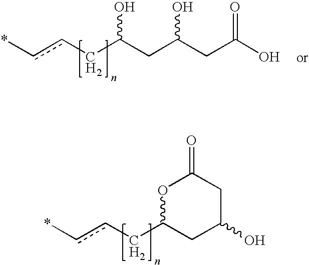

n being 0 or any integer;

R₂ is optionally substituted alkyl, cycloalkyl, aryl, or heteroaryl;

R₄ is optionally substituted

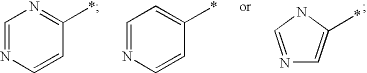

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXI:

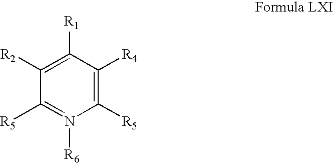

wherein R₁ is

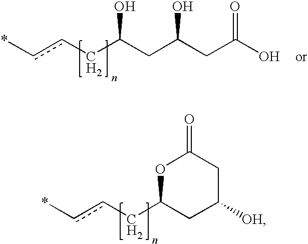

n being 0 or any integer;

R₂ is optionally substituted alkyl, cycloalkyl, aryl, or heteroaryl;

R₄ is optionally substituted

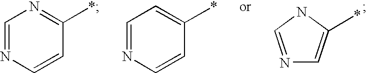

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXII:

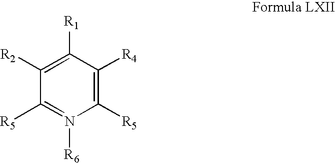

wherein R₁ is

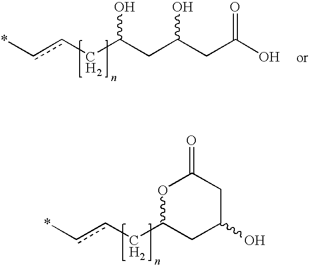

n being 0 or any integer;

R₂ is optionally substituted alkyl, cycloalkyl, aryl, or heteroaryl;

R₄ is optionally substituted

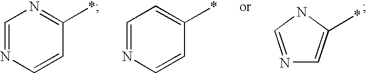

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXII:

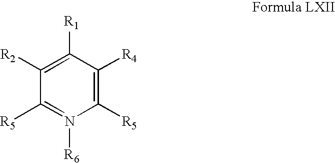

wherein R₁ is

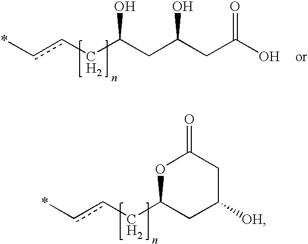

n being 0 or any integer;

R₂ is optionally substituted alkyl, cycloalkyl, aryl, or heteroaryl;

R₄ is optionally substituted

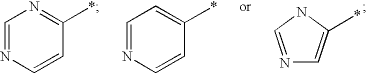

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXIII:

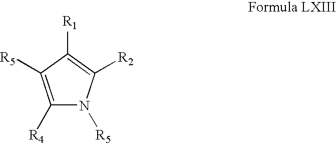

wherein R₁ is

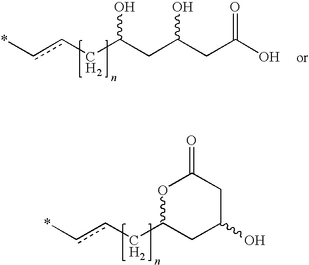

n being 0 or any integer;

R₂ is optionally substituted alkyl, cycloalkyl, aryl, or heteroaryl;

R₄ is optionally substituted

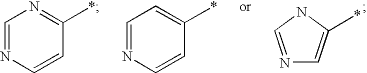

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXIII:

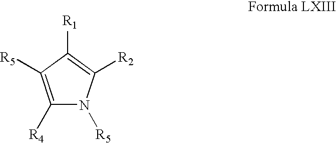

wherein R₁ is

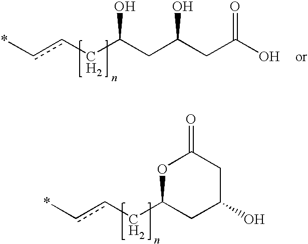

n being 0 or any integer;

R₂ is optionally substituted alkyl, cycloalkyl, aryl, or heteroaryl;

R₄ is optionally substituted

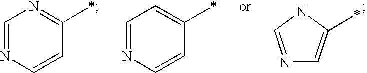

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXIV:

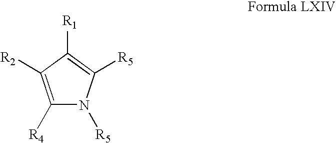

wherein R₁ is

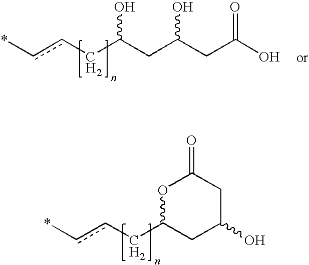

n being 0 or any integer;

R₂ is optionally substituted alkyl, cycloalkyl, aryl, or heteroaryl;

R₄ is optionally substituted

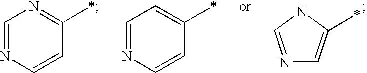

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXIV:

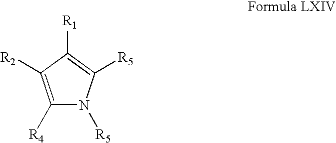

wherein R₁ is

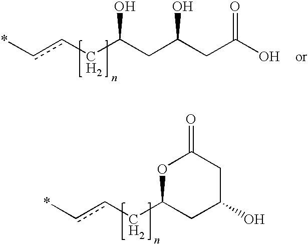

n being 0 or any integer;

R₂ is optionally substituted alkyl, cycloalkyl, aryl, or heteroaryl;

R₄ is optionally substituted

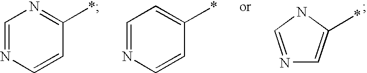

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXV:

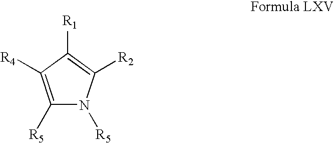

wherein R₁ is

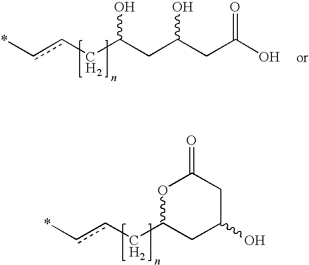

n being 0 or any integer;

R₂ is optionally substituted alkyl, cycloalkyl, aryl, or heteroaryl;

R₄ is optionally substituted

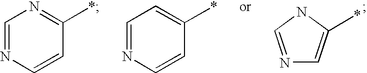

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXV:

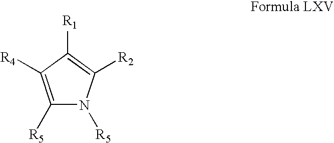

wherein R₁ is

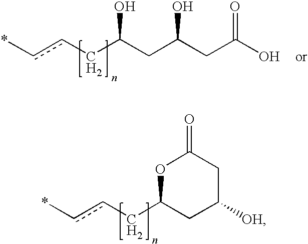

n being 0 or any integer;

R₂ is optionally substituted alkyl, cycloalkyl, aryl, or heteroaryl;

R₄ is optionally substituted

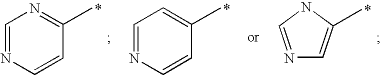

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXVII:

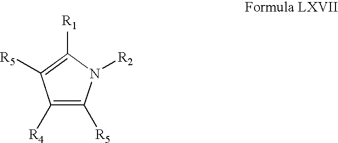

wherein R₁ is

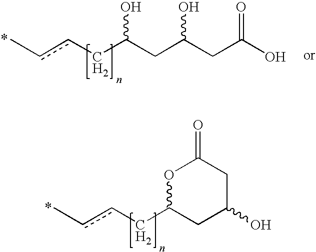

n being 0 or any integer;

R₂ is optionally substituted alkyl, cycloalkyl, aryl, or heteroaryl;

R₄ is optionally substituted

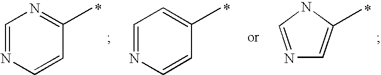

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXVII:

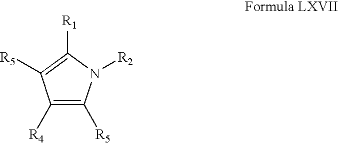

wherein R₁ is

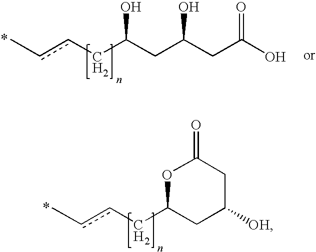

n being 0 or any integer;

R₂ is optionally substituted alkyl, cycloalkyl, aryl, or heteroaryl;

R₄ is optionally substituted

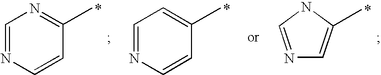

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXVIII:

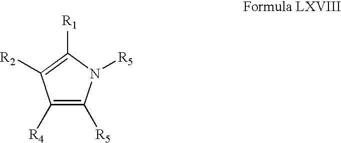

wherein R₁ is

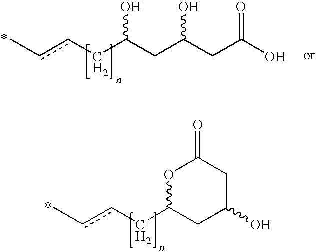

n being 0 or any integer;

R₂ is optionally substituted alkyl, cycloalkyl, aryl, or heteroaryl;

R₄ is optionally substituted

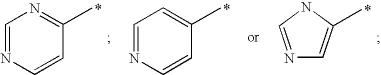

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXVIII:

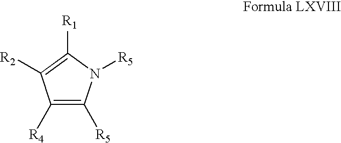

wherein R₁ is

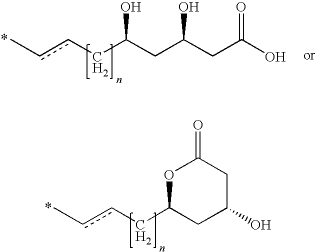

n being 0 or any integer;

R₂ is optionally substituted alkyl cycloalkyl, aryl, or heteroaryl;

R₄ is optionally substituted

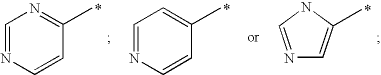

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXIX:

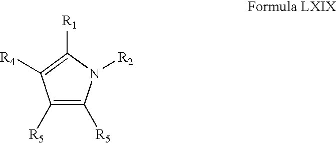

wherein R₁ is

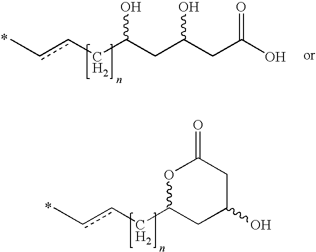

n being 0 or any integer;

R₂ is optionally substituted alkyl, cycloalkyl, aryl, or heteroaryl;

R₄ is optionally substituted

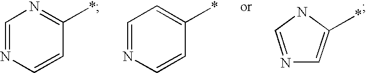

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXIX:

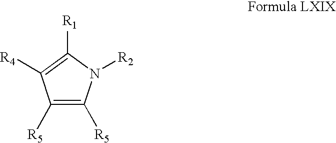

wherein R₁ is

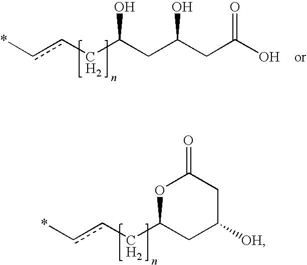

n being 0 or any integer;

R₂ is optionally substituted alkyl, cycloalkyl, aryl, or heteroaryl;

R₄ is optionally substituted

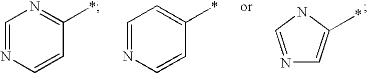

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXI:

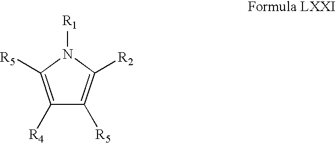

wherein R₁ is

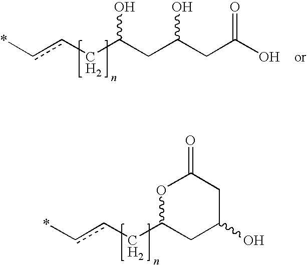

n being 0 or any integer;

R₂ is optionally substituted alkyl, cycloalkyl, aryl, or heteroaryl;

R₄ is optionally substituted

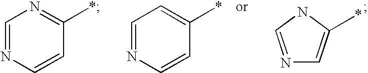

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXI:

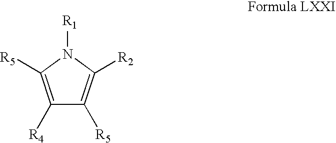

wherein R₁ is

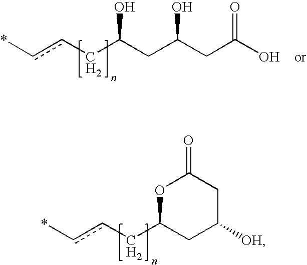

n being 0 or any integer;

R₂ is optionally substituted alkyl, cycloalkyl, aryl, or heteroaryl;

R₄ is optionally substituted

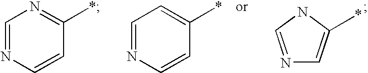

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXII:

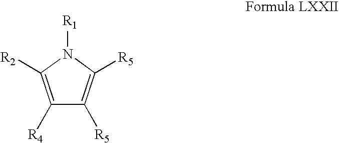

wherein R₁ is

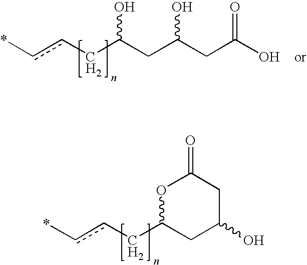

n being 0 or any integer;

R₂ is optionally substituted alkyl, cycloalkyl, aryl, or heteroaryl;

R₄ is optionally substituted

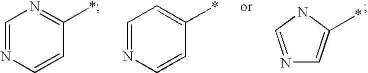

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXII:

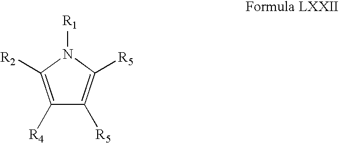

wherein R₁ is

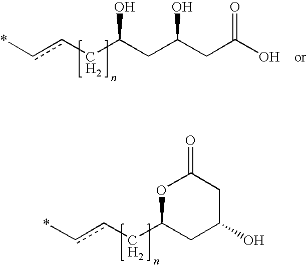

n being 0 or any integer;

R₂ is optionally substituted alkyl, cycloalkyl, aryl, or heteroaryl;

R₄ is optionally substituted

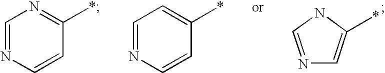

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXIII:

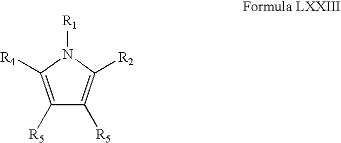

wherein R₁ is

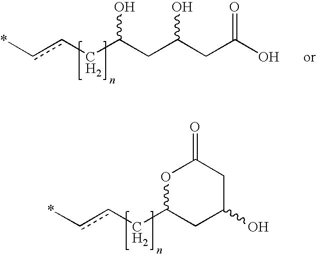

n being 0 or any integer;

R₂ is optionally substituted alkyl, cycloalkyl, aryl, or heteroaryl;

R₄ is optionally substituted

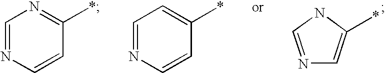

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXIII:

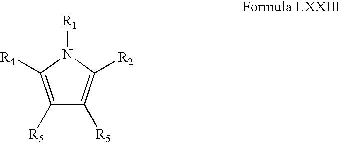

wherein R₁ is

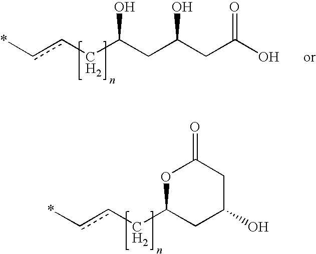

n being 0 or any integer;

R₂ is optionally substituted alkyl, cycloalkyl, aryl, or heteroaryl;

R₄ is optionally substituted

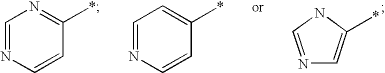

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXX:

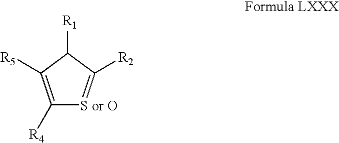

wherein R₁ is

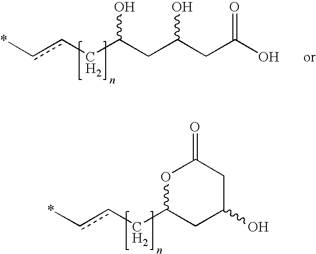

n being 0 or any integer;

R₂ is optionally substituted alkyl, cycloalkyl, aryl, or heteroaryl;

R₄ is optionally substituted

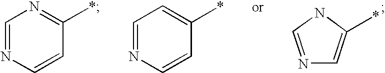

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXX:

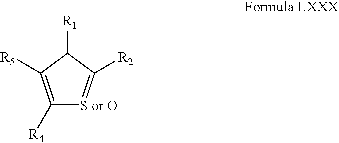

wherein R₁ is

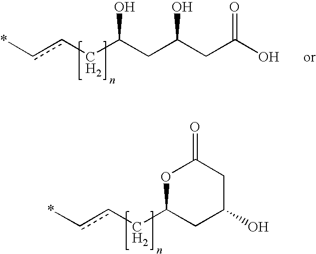

n being 0 or any integer;

R₂ is optionally substituted alkyl, cycloalkyl, aryl, or heteroaryl;

R₄ is optionally substituted

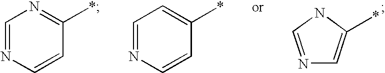

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXI:

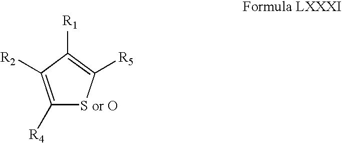

wherein R₁ is

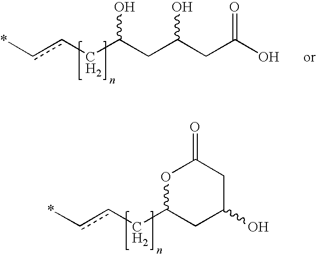

n being 0 or any integer;

R₂ is optionally substituted alkyl, cycloalkyl, aryl, or heteroaryl;

R₄ is optionally substituted

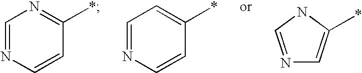

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXI:

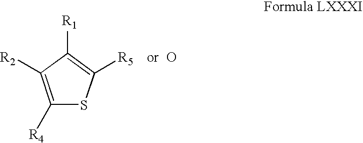

wherein R₁ is

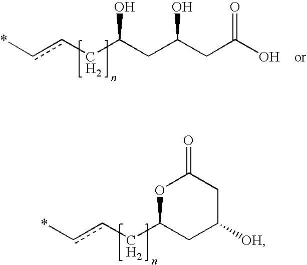

n being 0 or any integer;

R₂ is optionally substituted alkyl, cycloalkyl, aryl, or heteroaryl;

R₄ is optionally substituted

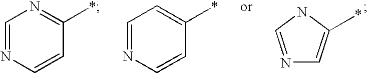

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXII:

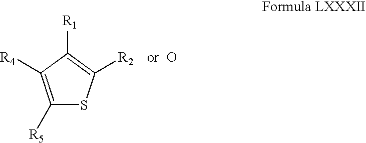

wherein R₁ is

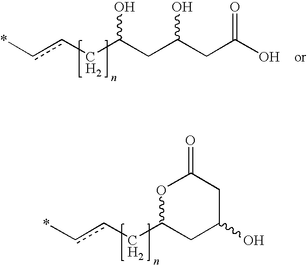

n being 0 or any integer;

R₂ is optionally substituted alkyl, cycloalkyl, aryl, or heteroaryl;

R₄ is optionally substituted

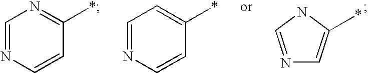

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXII:

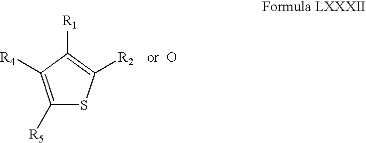

wherein R₁ is

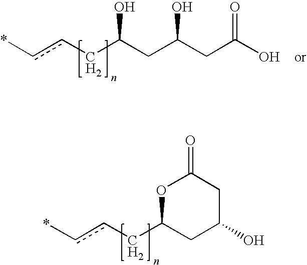

n being 0 or any integer;

R₂ is optionally substituted alkyl, cycloalkyl, aryl, or heteroaryl;

R₄ is optionally substituted

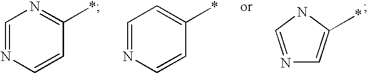

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXIV:

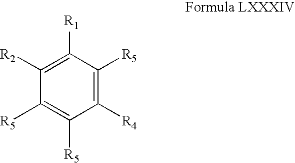

wherein R₁ is

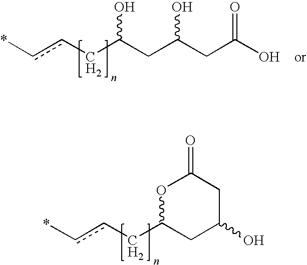

n being 0 or any integer;

R₂ is optionally substituted alkyl, cycloalkyl, aryl, or heteroaryl;

R₄ is optionally substituted

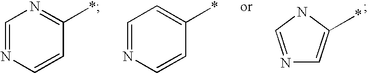

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXIV:

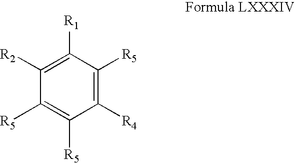

wherein R₁ is

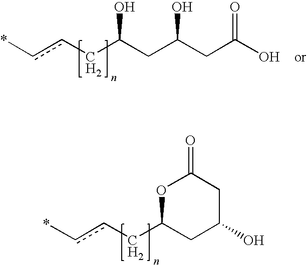

n being 0 or any integer;

R₂ is optionally substituted alkyl, cycloalkyl, aryl, or heteroaryl;

R₄ is optionally substituted

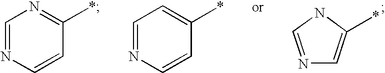

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXV:

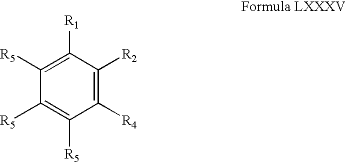

wherein R₁ is

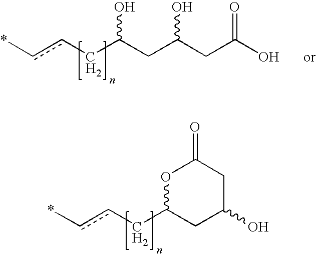

n being 0 or any integer;

R₂ is optionally substituted alkyl, cycloalkyl, aryl, or heteroaryl;

R₄ is optionally substituted

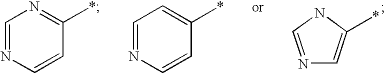

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXV:

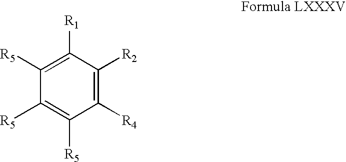

wherein R₁ is

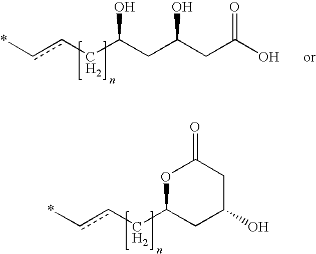

n being 0 or any integer;

R₂ is optionally substituted alkyl, cycloalkyl, aryl, or heteroaryl;

R₁ is optionally substituted

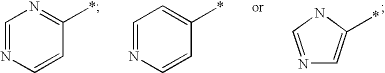

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXVI:

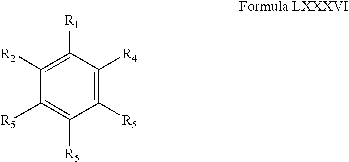

wherein R₁ is

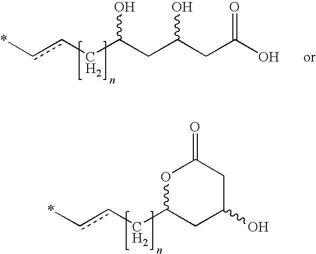

n being 0 or any integer;

R₂ is optionally substituted alkyl, cycloalkyl, aryl, or heteroaryl;

R₄ is optionally substituted

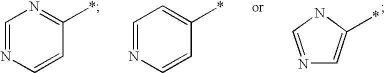

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXVI:

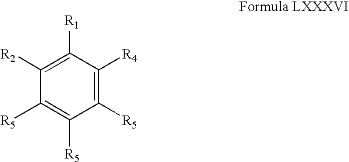

wherein R₁ is

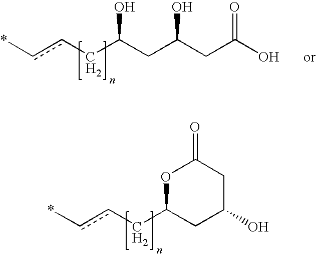

n being 0 or any integer;

R₂ is optionally substituted alkyl, cycloalkyl, aryl, or heteroaryl;

R₄ is optionally substituted

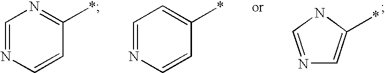

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXVII:

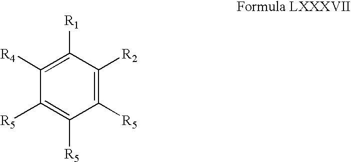

wherein R₁ is

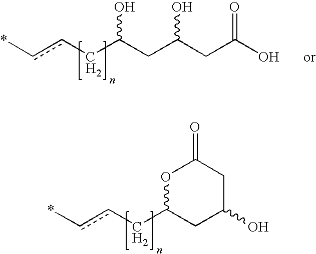

n being 0 or any integer;

R₂ is optionally substituted alkyl, cycloalkyl, aryl, or heteroaryl;

R₄ is optionally substituted

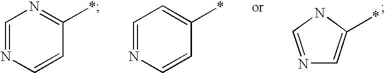

and R₅ is m each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXVII:

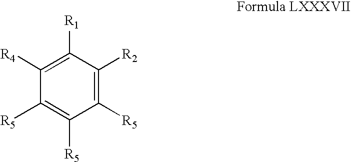

wherein R₁ is

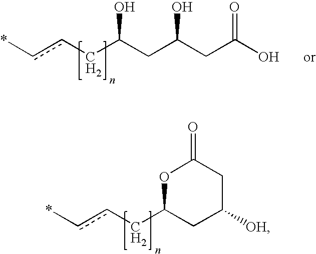

n being 0 or any integer;

R₂ is optionally substituted alkyl, cycloalkyl, aryl, or heteroaryl;

R₄ is optionally substituted

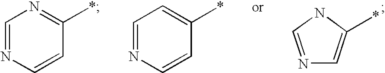

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXVIII:

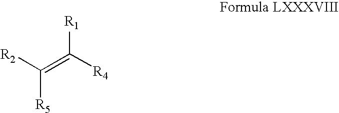

wherein R₁ is

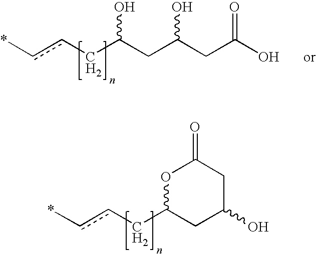

n being 0 or any integer;

R₂ is optionally substituted alkyl, cycloalkyl, aryl, or heteroaryl;

R₄ is optionally substituted

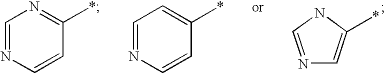

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXVIII:

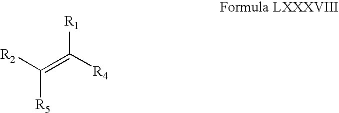

wherein R₁ is

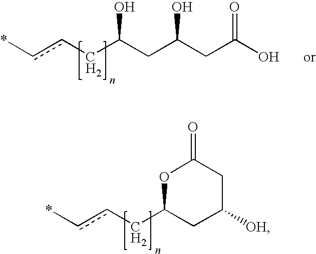

n being 0 or any integer;

R₂ is optionally substituted alkyl, cycloalkyl, aryl, or heteroaryl;

R₄ is optionally substituted

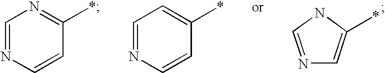

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXIX:

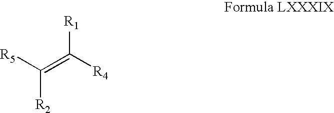

wherein R₁ is

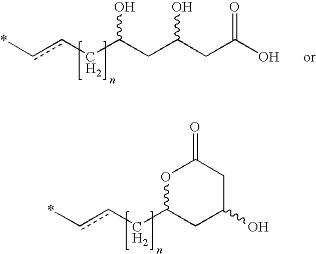

n being 0 or any integer;

R₂ is optionally substituted alkyl, cycloalkyl, aryl, or heteroaryl;

R₄ is optionally substituted

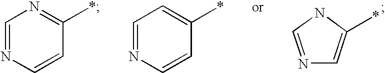

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXIX:

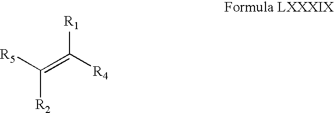

wherein R₁ is

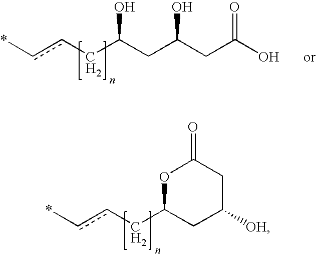

n being 0 or any integer;

R₂ is optionally substituted alkyl, cycloalkyl, aryl, or heteroaryl;

R₄ is optionally substituted

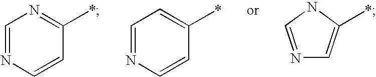

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXX:

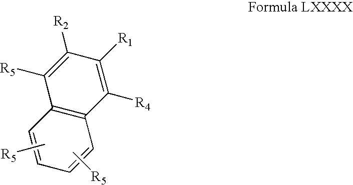

wherein R₁ is

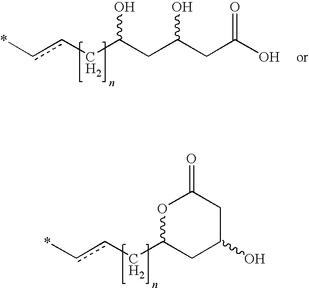

n being 0 or any integer;

R₂ is optionally substituted alkyl, aryl, or heteroaryl;

R₄ is optionally substituted

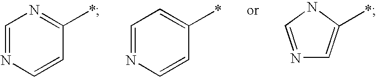

and R₅ is in each instance hydrogen, optionally substituted lower alkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXX:

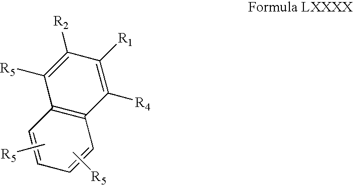

wherein R₁ is

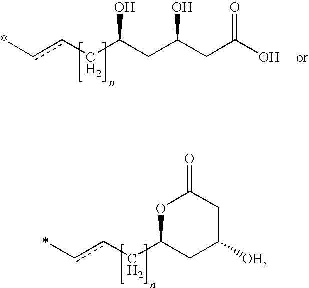

n being 0 or any integer;

R₂ is optionally substituted alkyl, cycloalkyl, aryl, or heteroaryl;

R₄ is optionally substituted

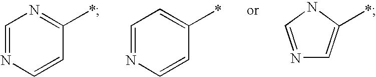

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXI:

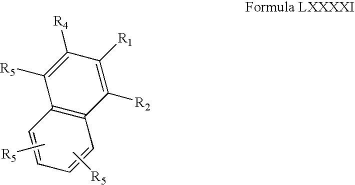

wherein R₁ is

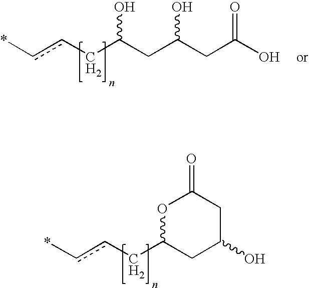

n being 0 or any integer;

R₂ is optionally substituted alkyl, cycloalkyl, aryl, or heteroaryl;

R₄ is optionally substituted

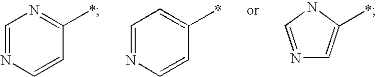

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXXI:

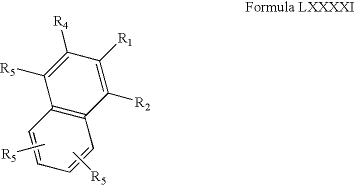

wherein R₁ is

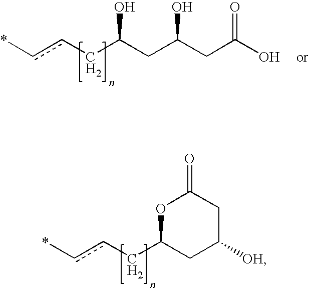

n being 0 or any integer;

R₂ is optionally substituted alkyl, cycloalkyl, aryl, or heteroaryl;

R₄ is optionally substituted

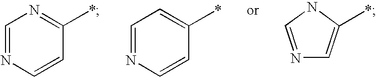

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXII:

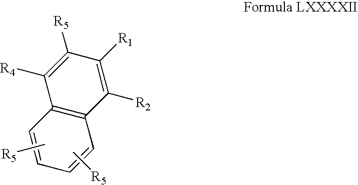

wherein R₁ is

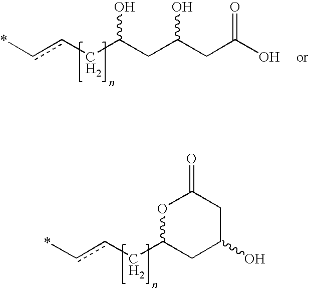

n being 0 or any integer;

R₂ is optionally substituted alkyl, cycloalkyl, aryl, or heteroaryl;

R₄ is optionally substituted

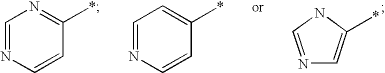

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXXII:

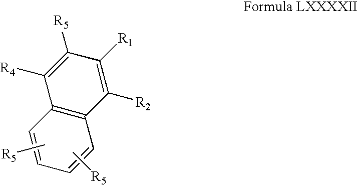

wherein R₁ is

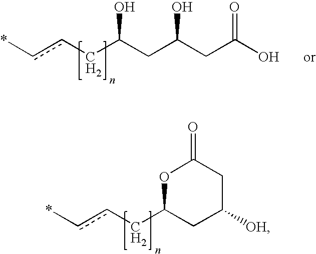

n being 0 or any integer;

R₂ is optionally substituted alkyl, cycloalkyl, aryl, or heteroaryl;

R₄ is optionally substituted

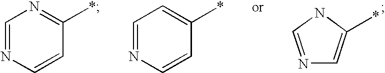

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXIII:

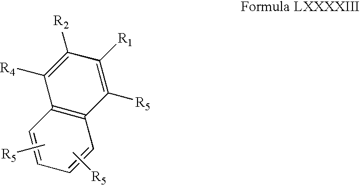

wherein R₁ is

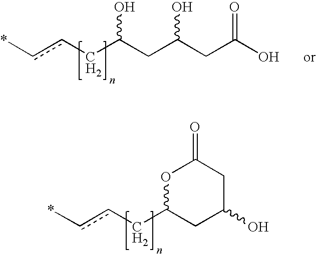

n being 0 or any integer;

R₂ is optionally substituted alkyl, cycloalkyl, aryl, or heteroaryl;

R₄ is optionally substituted

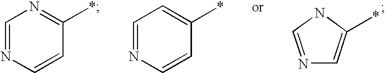

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXXIII:

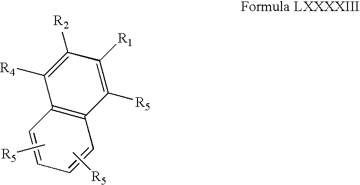

wherein R₁ is

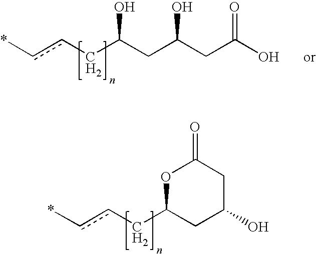

n being 0 or any integer;

R₂ is optionally substituted alkyl, cycloalkyl, aryl, or heteroaryl;

R₄ is optionally substituted

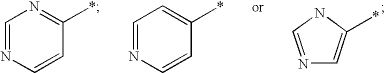

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXIV:

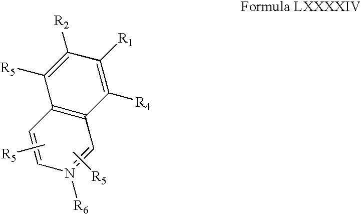

wherein R₁ is

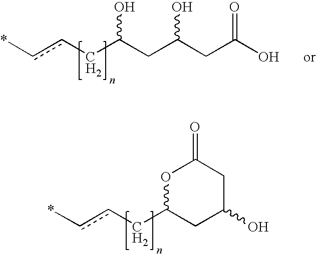

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXIV:

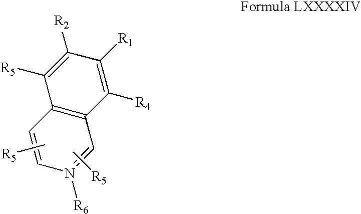

wherein R₁ is

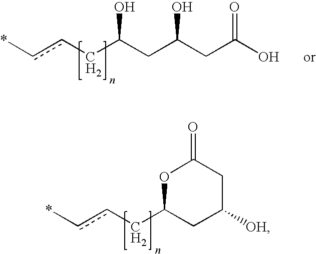

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXV:

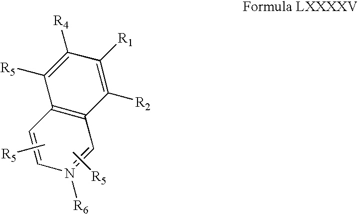

wherein R₁ is

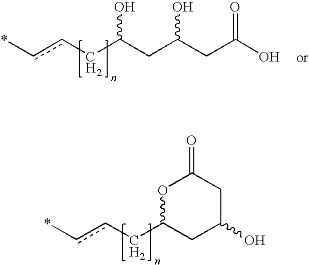

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXV:

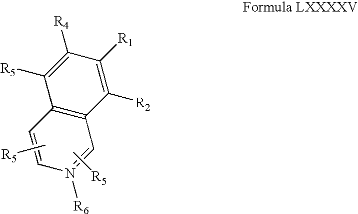

wherein R₁ is

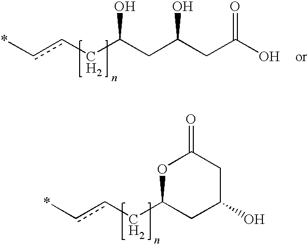

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXVI:

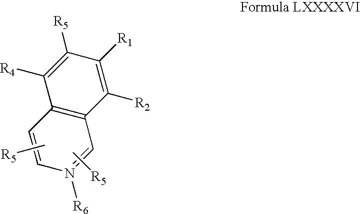

wherein R₁ is

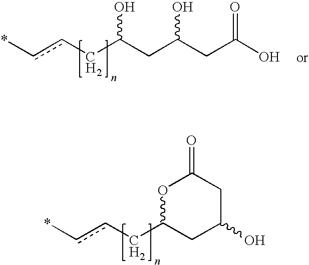

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXVI:

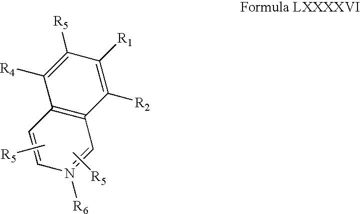

wherein R₁ is

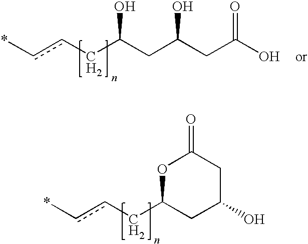

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXVII:

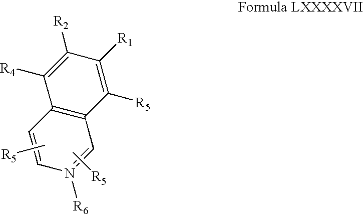

wherein R₁ is

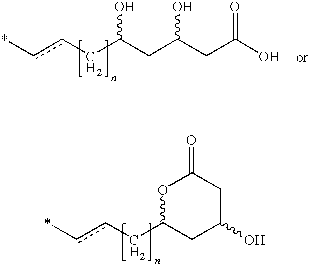

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXVII:

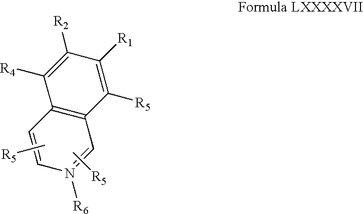

wherein R₁ is

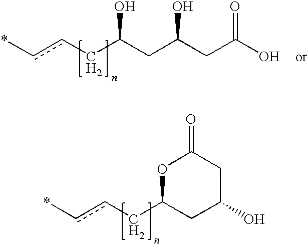

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXVIII:

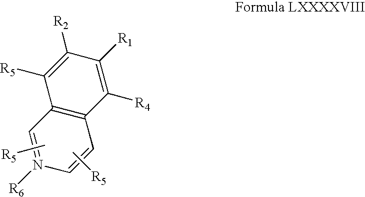

wherein R₁ is

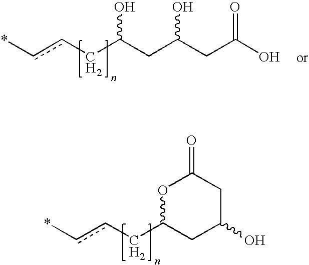

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXVIII:

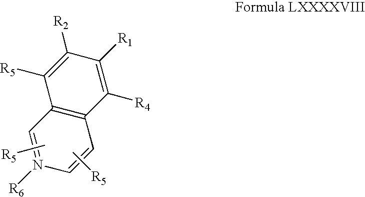

wherein R₁ is

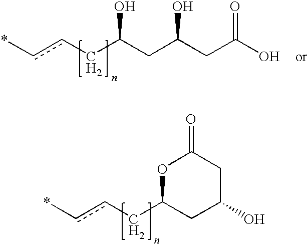

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXIX:

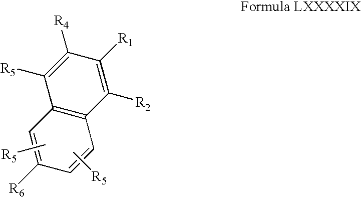

wherein R₁ is

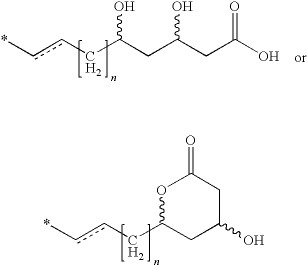

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXIX:

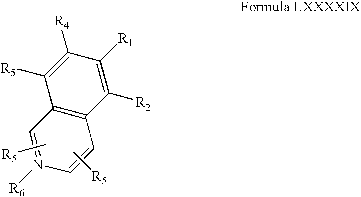

wherein R₁ is

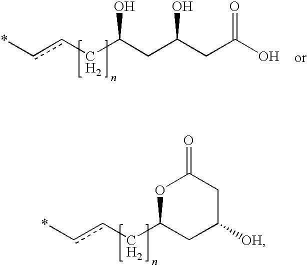

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula C:

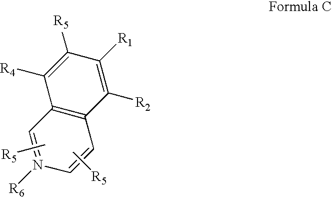

wherein R₁ is

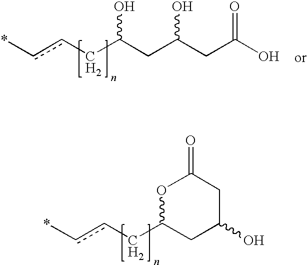

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted ammo; and

R₆ is either a lone pair of electrons or an oxygen atom.

Another aspect of the present invention provides a compound comprising formula C:

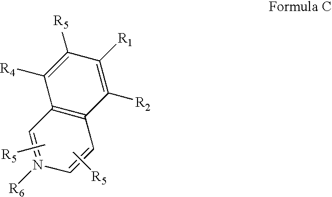

wherein R₁ is

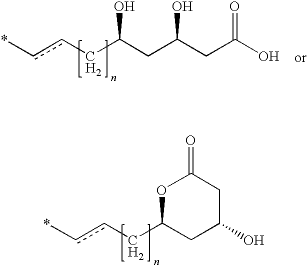

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula CI:

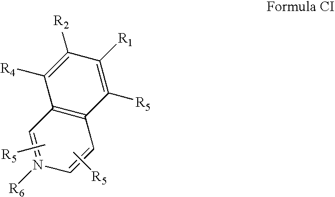

wherein R₁ is

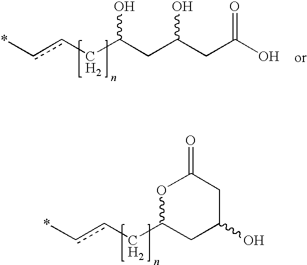

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula CI:

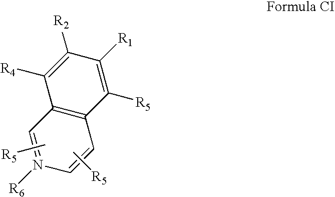

wherein R₁ is

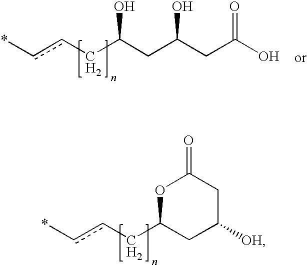

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula CII:

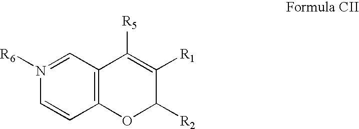

wherein R₁ is

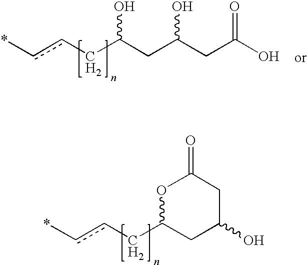

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula CII:

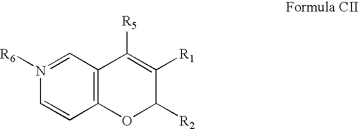

wherein R₁ is

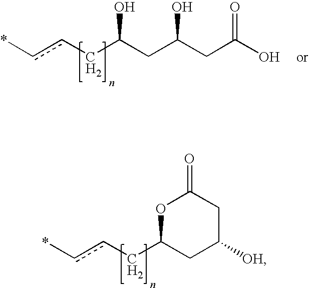

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, optionally substituted amino; and

R₆ is either a lone pair of electrons or an oxygen atom.

One aspect of the present invention provides methods of inhibiting a MAP kinase comprising administering an effective amount of at least one compound comprising formula CIII:

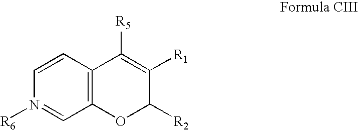

wherein R₁ is

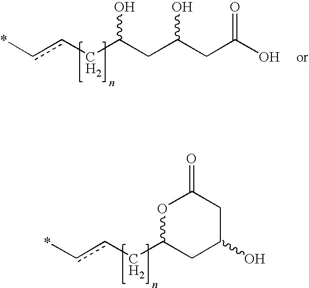

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula CIII:

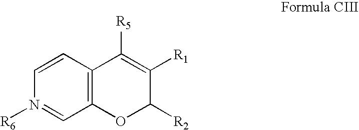

wherein R₁ is

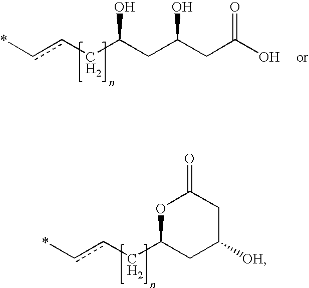

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

In some embodiments, the inhibited MAP kinase is p38 MAP kinase. In some embodiments, the method further comprises inhibiting an HMG CoA reductase. In some embodiments, the administering treats a MAP kinase-related condition. In some embodiments, the administering treats a MAP kinase-related condition and an HMG CoA reductase-related condition. In some embodiments, the administering treats an inflammatory condition.

Still another aspect of the instant invention provides a pharmaceutical composition comprising an effective amount of at least one compound as recited above with a pharmaceutically acceptable carrier.

Still other aspects of the instant invention provide methods of treating a condition in a subject in need thereof comprising administering to the subject an effective amount of at least one compound as recited above.

### BRIEF DESCRIPTION OF THE FIGURES

FIG. 1 illustrates some of the pathways involved in inflammatory signaling cascades and the interruption of certain of these pathways by a MAP kinase inhibitor.

FIG. 2a illustrates some of the pathways involved in cholesterol biosynthesis and some of the atherogenic mechanisms of hypercholesterolemia, as well as the interruption of certain of these pathways by an HMG-CoA reductase inhibitor.

FIG. 2b illustrates some of the pathways involved in processing amyloid precursor protein, the role played by cholesterol in such pathways, as well as the interruption of certain of these pathways by an HMG-CoA reductase inhibitor.

FIG. 3 illustrates a treatment approach in which compositions of the present invention produce a benefit in both MAP kinase-related and HMG-CoA reductase-related conditions.

FIG. 4 illustrates certain preferred compounds of the present invention.

FIG. 5 illustrates certain preferred compounds of the present invention.

FIG. 6 illustrates certain preferred compounds of the present invention.

FIG. 7 illustrates certain preferred compounds of the present invention.

### DETAILED DESCRIPTION OF THE INVENTION

### I. Kinase and/or HMG-CoA Reductase Inhibitors

One aspect of the present invention relates to compounds that inhibit protein kinases, e.g., protein kinases involved in inflammatory signaling cascades. In some embodiments, these compounds can inhibit mitogen-activated protein kinases (MAP kinases). For example, these compounds can inhibit p38 MAP kinases and/or stress-activated protein kinases/Jun N-terminal kinases (SAPKs/JNKs). In some embodiments, these compounds can inhibit p38αMAP kinase. In preferred embodiments, such compounds exert anti-inflammatory effects in vitro and in vivo, e.g., as described in more detail below.

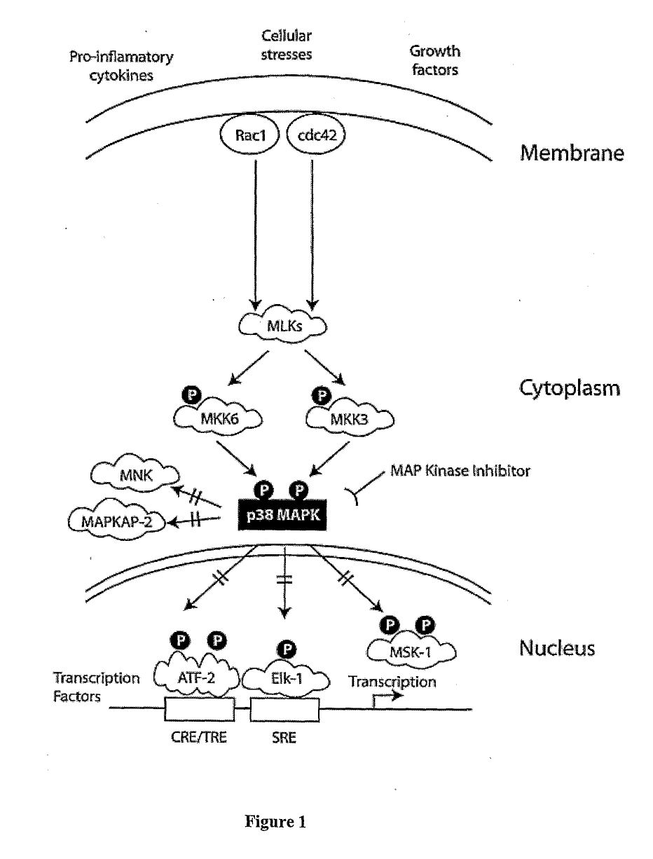

FIG. 1 illustrates some of the pathways involved in inflammatory signaling cascades and the interruption of certain of these pathways by a MAP kinase inhibitor. This figure provides an overview only, and is in no way intended to be limiting with respect to the present invention. For example, those skilled in the art will readily appreciate variations and modifications of the scheme illustrated.

As FIG. 1 illustrates, inflammatory signaling cascades transmit signals from outside a cell membrane 101 to the cytoplasm 102 and ultimately the nucleus 103. Pro-inflammatory cytokines 104 (e.g., TNF-α and IL-1), as well as cellular stresses 105 and growth factors 106, initiate a signal transduction cascade leading to the activation of several serine/threonine kinases, including MKK3, MKK6 and p38 MAP kinase. Chakravarty et al, Annual Reports in Medicinal Chemistry, Chapter 18, Elsevier Science (2002). As is known in the art, p38 MAP kinases exist in at least four isoforms, p38α (expressed in all tissues), p38β (expressed in all tissues), p38γ (primarily expressed in skeletal tissue), and p38δ (primarily expressed in the lungs, kidneys, testes, pancreas and small intestine). One or more of these MAP kinases can be inhibited by a compound of the instant invention, or a combination comprising one or more such compounds, e.g., by interaction with their shared Thr-Gly-Tyr dual phosphorylation activation motif and/or with their highly conserved amino acid sequences, e.g., the conserved binding pocket for ATP. In preferred embodiments, p38αMAP kinase is inhibited, p38αMAP kinase serving as the primary MAP kinase associated with the pro-inflammatory cytokines. As such, p38αMAP kinase presents a target for small molecule therapeutics aimed at reducing cytokine production and treating associated inflammatory and/or autoimmune conditions.

As FIG. 1 illustrates, activation of p38 MAP kinase by upstream kinases leads to phosphorylation of downstream substrates, including MNK and MAPKAP-2, as well as transcription factors ATF-2, Elk-1, and MSK-1, which control transcription and production of pro-inflammatory cytokines. FIG. 1 also illustrates points of action of an inhibitor that can reduce downstream effects of p38 MAP kinase, illustrated by double bars. For example, inhibition of p38αMAP kinase using a compound of the present invention, or a composition comprising one or more such compounds, can reduce phosphorylation of MNK, MAPKAP-2, ATF-2, Elk-1 and/or MSK-1, reducing production of pro-inflammatory cytokines, in certain embodiments, as discussed in detail below.

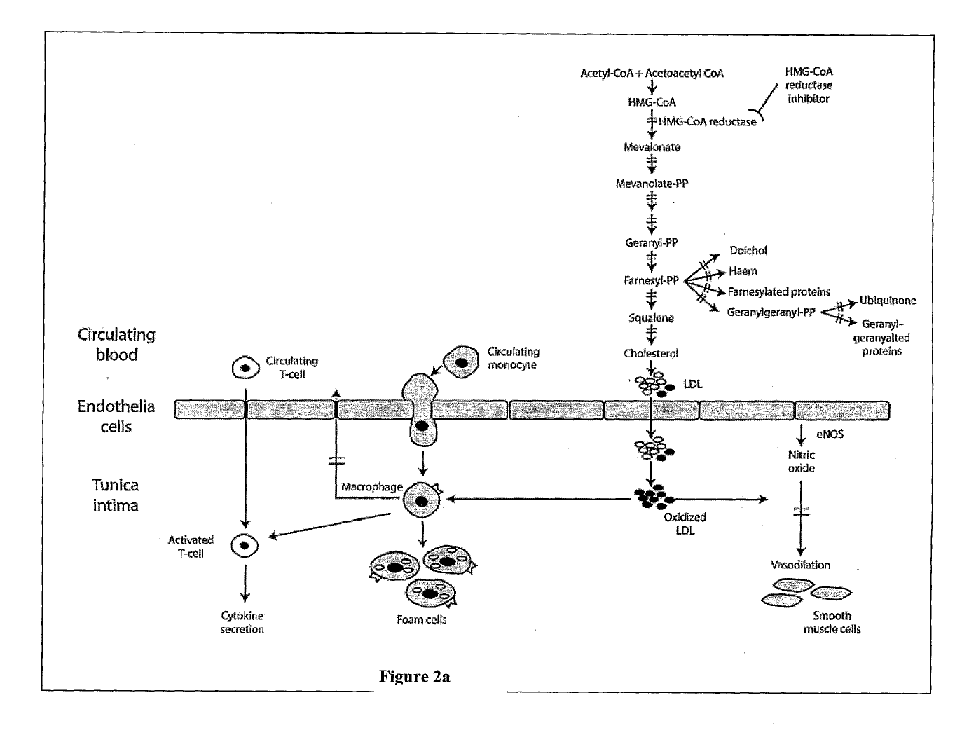

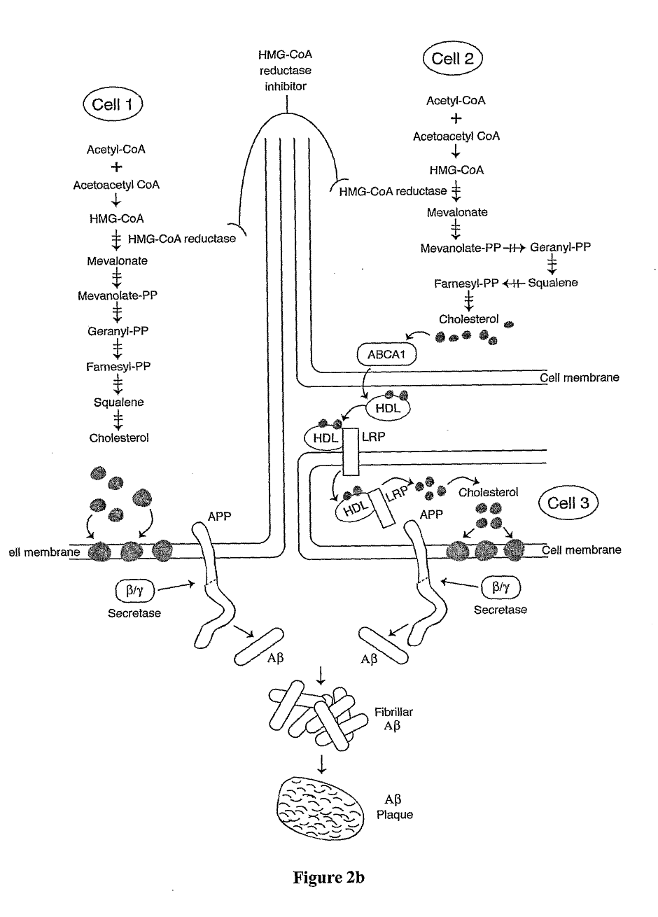

A second aspect of the present invention relates to compounds that can inhibit the enzyme 3-hydroxy-3-methyl glutaryl-coenzyme A reductase (HMG-CoA reductase). These compounds can lower cholesterol levels in vitro and in vivo. FIG. 2a illustrates some of the pathways involved in cholesterol biosynthesis and some of the atherogenic mechanisms of hypercholesteremia, as well as the interruption of certain of these pathways by an HMG-CoA reductase inhibitor. This figure provides an overview only, and is in no way intended to be limiting. For example, those skilled in the art will readily appreciate variations and modifications of the scheme illustrated, and more detailed descriptions can be found in standard texts on biochemistry, metabolism, pathophysiology, and the like.

As is known in the art, HMG-CoA reductase catalyzes the committed, rate-limiting step of terpene and cholesterol synthesis in mammalian cells. It thus represents a target for small molecule therapeutics (e.g., the “statins”) aimed at reducing atherogenesis and its associated cardiovascular risks. HMG-CoA reductase acts on 3-hydroxy-3-methyl-glutaryl CoA (HMG-CoA) to produce mevalonate. Mevalonate is converted into cholesterol, which is carried in the blood mainly in two specialized particles known as low-density lipoprotein (LDL) and high-density lipoprotein (HDL). The pathway also produces other non-sterol isoprenoid products, such as farnesol, dolichol, and ubiquinone.

As illustrated in FIG. 2a, LDL adheres to the arterial wall and is progressively oxidized. Palinski et al., J. Am. Soc. Nephrol., 13: 1673-1681 (2002). Extensively oxidized LDL is taken up by macrophages to form foam cells, a key feature of atherosclerosis. This leads to recruitment of monocytes and T-cells and secretion of cytokines in immune response cascades. The double bars indicate currently known effects of HMG-CoA reductase inhibitors (e.g., statins) on these processes, not only in reducing the production of cholesterol, but also in modulating immune responses through the actions of other metabolites such as farnesyl pyrophosphate and geranylgeranyl pyrophosphate. For example, geranylgeranyl-PP decreases endothelial cell nitric oxide synthase (eNOS) expression, inhibiting nitric oxide-induced vasodilation. Inhibition of HMG CoA reductase using a compound of the present invention, or a composition comprising one or more such compounds, can also produce these effects, in certain embodiments, as discussed in detail below.

A compound of the present invention, or a composition comprising one or more such compounds, can increase HDL levels (“good cholesterol”) in some embodiments. HDL plays a role in carrying excess cellular cholesterol in what is known as the reverse cholesterol transport pathway. Generally, HDL is a complex of protein, lipids and cholesterol, which “scours” the walls of blood vessels to remove excess cholesterol. In reverse cholesterol transport, peripheral tissues (e.g., vessel-wall macrophages) remove excess cholesterol through ABCA1 to apolipoprotein A-I, forming pre-β-HDL. Lecithin-cholesterol acyltransferase then esterifies free cholesterol to cholesteryl esters, converting pre-β-HDL to mature spherical α-HDL. Forrester, J. S., Makkar, R., Shah, P. K. Circulation 111: 1847-1854 (2005), incorporated herein by reference. A compound of the present invention, or a combination comprising one or more such compounds, can decrease serum LDL/HDL ratios, in some embodiments.

Several steps in the cholesterol biosynthesis pathway have been implicated in Alzheimer's disease-related processes. Alzheimer's has been linked to several proteins of the cholesterol biosynthesis pathway. As is known in the art, neuronal cells obtain cholesterol in two ways: through de novo synthesis or by internalization through endosomal mechanisms. Cells which utilize the former synthesize cholesterol de novo in the endoplasmic reticulum and thereafter transport it to the cell membrane. Cells that utilize the latter internalize cholesterol synthesized by other neuronal cells such as astrocytes. For example, cholesterol secreted via the ATP-binding cassette transporter 1 (ABCA1) transporter protein is taken up by brain HDL, containing apoliproteins E and J. Cholesterol-containing brain HDL can be internalized by neuronal cells through an extracellular membrane receptor, called low-density lipoprotein-related receptor (LRP). Uptake is further assisted by LRP8 and very-low-density lipoprotein receptor (VLDLR). Polymorphisms in genes encoding cholesterol pathway proteins are putative risk factors for Alzheimer's. Such cholesterol pathway proteins include, e.g., the transport molecule apolipoprotein E, the uptake molecules LRP, LRP8, and VLDLR, as well as ABCA1 (a catabolism-related molecule), and Cyp46 (an oxysterol producer). Wolozin, W., Cholesterol, statins and dementia (review), Curr. Op. Lipidol. 15:667-672 (2004).

The pathology of Alzheimer's disease is characterized by the presence of neuritic plaques composed largely of β-amyloid (Aβ) protein fragments. Aβ is produced when membrane bound amyloid precursor protein (APP) is cleaved by proteolytic enzymes, β-secretase and γ-secretase. Soluble Aβ fragments cluster with one another to form oligomers, then fibrillar Aβ aggregates, and eventually neuritic Aβ plaques.

FIG. 2b illustrates some of the pathways involved in processing amyloid precursor protein, the role played by cholesterol in such pathways, as well as the interruption of certain of these pathways by an HMG-CoA reductase inhibitor. This figure provides an overview only, and is in no way intended to be limiting. For example, those skilled in the art will readily appreciate variations and modifications of the scheme illustrated, and more detailed descriptions can be found in standard texts on biochemistry, metabolism, pathophysiology, and the like.

As shown in FIG. 2b, a cholesterol-rich membrane is required for proteolysis of APP, which subsequently leads to the production of Aβ and eventual Aβ plaque formation. Wolozin, W., Cholesterol, statins and dementia (review), Curr. Op. Lipidol. 15:667-672 (2004). Cell 1 of FIG. 2b shows a neuronal cell in the process of synthesizing its own cholesterol de novo and then transporting it to the cell membrane to allow APP processing. Cell 2 shows a neuronal cell in the process of synthesizing and secreting it through the ABCA1 transporter protein where brain HDL protein binds cholesterol. Cell 3 shows a neuronal cell in the process of internalizing the HDL-cholesterol complex by way of LRP. The subsequent transport of cholesterol to Cell 3's membrane allows APP processing to occur. FIG. 2b also illustrates how inhibition of HMG CoA reductase in Cell 1 and Cell 2 using an HMG CoA reductase inhibitor can produce an inhibitory effect on cholesterol synthesis and thereby affect APP processing. The double bars indicate currently known effects of HMG-CoA reductase inhibitors (e.g., statins) on these processes. For example, HMG-CoA reductase inhibitors have been found to reduce β-secretase proteolysis of APP in cultured human cells overexpressing APP, while applying solubilized cholesterol to such cells resulted in a significant increase in Aβ products. In addition, reducing cellular cholesterol levels in hippocampal neurons has been shown to inhibit Aβ formation. Reiss, A. B. et al., Cholesterol in neurologic disorders of the elderly: stroke and Alzheimer's disease (review), Neurobiology of Aging 25:977-89 (2004). Inhibition of HMG CoA reductase using a compound of the present invention, or a composition comprising one or more such compounds, can also produce these effects, in certain embodiments, as discussed in detail below.

A third aspect of this invention relates to compounds that inhibit both MAP kinase and HMG-CoA reductase activities. Such compounds can inhibit both inflammatory responses and cholesterol biosynthetic pathways in vitro and in vivo, and can exert, for example, anti-inflammatory, lipid-modulating, and anti-atherogenic properties in vivo. Further, such compounds can provide superior benefits in treating HMG-CoA reductase-related conditions, such as cardiovascular disease, compared with treatments that inhibit HMG-CoA reductase but not MAP kinase, due to the interplay between inflammatory and cardiovascular disorders. In other embodiments, such compounds can provide superior benefits in treating MAP kinase-related conditions, such as inflammation, compared with treatments that inhibit MAP kinase but not HMG-CoA reductase, again due to the interplay between inflammatory and cardiovascular conditions.

A fourth aspect of this invention relates to combinations of two or more compounds or forms of compounds that inhibit MAP kinase and/or HMG-CoA reductase activities, e.g., to produce one or more of the effects described above. Such combinations find particular use in treating inflammatory conditions. Without being limited to a particular hypothesis, theory or mechanism, HMG-CoA reductase and MAP kinase may both play a role in certain inflammatory conditions, making the use of combination therapies particularly effective. For example, HMG-CoA reductase and MAP kinase both have been implicated in inflammatory conditions of the skin.

Acne is an example of a skin inflammatory condition involving activities of both MAP kinase and HMG-CoA reductase. Acne results from the formation of a comedone followed by pericomedonian inflammation (or folliculitis). A comedone (or blackhead) forms when a pilo-sebaceous duct is obstructed and/or when there is increased production of sebum by a sebaceous gland. Formation of the comedone is followed by inflammation, e.g., resulting from bacterial proliferation due to seborrhoeic retention and/or overproduction of sebum. Typically, the bacteria are diphtheroid anaerobic bacteria such as Propionibacteria (acnes, granulosum, avidum). In addition to inflammatory pathways, pathways involving HMG-CoA reductase may also be involved. For example, it is known in the art that cholesterol and the metabolites thereof play a role in cohesion of epidermal cells, particularly corneocytes (cells constituting the stratum corneum).

As another example, psoriasis is a chronic hyperproliferative skin condition wherein the subject exhibits inflammation, as well as excess proliferation of epidermal cells (scaling). The cause is thought to be an abnormal immune response to some element of the skin prompted by malfunctioning T cells. It is known in the art that multiple cellular events occur at the response site including increased cell adhesion molecule expression, upregulation of cytokines and growth factors, and penetration of the tissue by lymphocytes. It is also known in the art that HMG-CoA reductase inhibitors downregulate expression of cell adhesion molecules, inhibit the interaction between adhesion molecules required for leukocyte infiltration into inflammation sites, suppress the expression of T-helper-1 chemokine receptors on T cells, and inhibit the expression of proinflammatory cytokines. Namazi, M. R., Experimental Dermatology, 13:337-39 (2004). As another example, is known in the art that a form of eczema, atopic dermatitis, is mediated by the inflammatory mediators IFN-γ and/or TNF-α.

HMG-CoA reductase and/or MAP kinase may also play a role in muscoskeletal inflammatory conditions, such as arthritis, psoriatic arthritis, osteoarthritis, rheumatoid arthritis, and osteoporosis. For example, pathological bone resorption or erosion in osteoporosis and rheumatoid arthritis requires the activation of osteoclasts (large multinucleate cells formed from differentiated macrophages) and TNF-α; IFN-γ and IL-1 have been implicated in triggering excess osteoclast activity. Roux, S. Bone loss. Factors that regulate osteoclast differentiation: an update (review), Arthritis Res., 2(6):451-456 (2000); Evans et al., Nitric oxide and bone (review), J Bone Miner Res. March; 11(3):300-5 (1996).

HMG-CoA reductase and/or MAP kinase may also play a role in respiratory inflammatory conditions. For example, the inflammatory mediators IFN-γ and/or TNF-α are known in the art to mediate asthma and mucocutaneous inflammatory conditions such as allergic rhinitis.

HMG-CoA reductase and/or MAP kinase may also play a role in gastrointestinal and urinogenital inflammatory conditions. For example, gastrointestinal inflammatory conditions, such as inflammatory bowel disease (including ulcerative colitis and Crohn's disease), celiac disease, intestinal infections, enterocolitis, and gastritis, exhibit chronic spontaneous relapsing enteropathies mediated by IFN-γ and TNF-α. Further, it is known in the art that urogenital inflammatory disorders are mediated by IFN-γ and TNF-α.

HMG-CoA reductase and/or MAP kinase may also play a role in autoimmune diseases. For example, according to recent reports, HMG-CoA reductase inhibitors may have a beneficial effect on autoimmune disorders, such as multiple sclerosis (MS). Stuve, O., et al., The potential therapeutic role of statins in central nervous system autoimmune disorders (review), Cell Mol Life Sci. 2003 November; 60(11):2483-91. Generally, MS is mediated by proinflammatory CD4 T (Th1) cells that recognize specific myelin proteins associated with MHC class II molecules on antigen presenting cells (APCs). It is known in the art that inhibitors of HMG-CoA reductase inhibit the production of iNOS, TNF-α, IL-1beta and IL-6 by microglia and astrocytes, both APCs. HMG-CoA reductase inhibitors also inhibit IFN-γ-inducible class II expression on APCs, e.g., by inhibiting transcription of the IFN-γ-inducible promoter, which may result in suppression of antigen presentation by APCs. Some HMG-CoA reductase inhibitors also bind lymphocyte function-associated antigen-1 (LFA-1), a beta2-integrin and prevent interaction with its ligand, ICAM-1, as well as T cell activation, suggesting a beneficial effect on MS independent of an inhibition of HMG-CoA reductase.

HMG-CoA reductase and/or MAP kinase may also play a role in graft rejection after organ or tissue transplantation. For example, HMG-CoA reductase inhibitors have been shown to significantly reduce the incidence of organ rejection, transplant vasculopathy, and natural killer (NK) cell cytotoxicity in recipients of heart transplants (Kobashigawa et al., Dual roles of HMG-CoA reductase inhibitors in solid organ transplantation: lipid lowering and immunosuppression (review), Kidney Int. Suppl., December; 52:S112-5 (1995)) and kidney transplants (Katznelson, S. et al., The effect of pravastatin on acute rejection after kidney transplantation—a pilot study (review), Transplantation, May 27; 61(10):1469-74 (1997)). Additionally, such inhibitors have been shown to decrease the progression of transplant vasculopathy and to increase patient survival (Wenke, K. et al., Simvastatin reduces graft vessel disease and mortality after heart transplantation: a four-year randomized trial, Circulation, September 2; 96(5):1398-402. (1997)), suggesting a possible drug class effect. It is known in the art that treatment of heart and kidney transplant patients with HMG-CoA reductase inhibitors significantly inhibits NK cell cytotoxicity beyond that obtained with the baseline regimen, consisting of prednisone, azathioprine, and cyclosporine. For example, it is known in the art that clinically relevant concentrations of simvastatin, which are not immunosuppressive themselves, significantly enhance inhibition of human T-cell responses by cyclosporin A in vitro. It has been suggested that synergism between the inhibitors and cyclosporin A could potentially be the basis for the immunosuppression uniquely observed in transplant patients. Katznelson, S. et al., Effect of HMG-CoA reductase inhibitors on chronic allograft rejection (Review), Kidney Int Suppl. 1999 July; 71:S117-21 (1999).

Accordingly, inhibition of HMG CoA reductase and/or MAP kinase, preferably inhibition of both, by a combination of compounds or forms of compounds of the present invention can also produce the aforementioned effects, in certain embodiments, as discussed in detail below.

Further, those of skill in the art will recognize that certain compounds of the present invention may exhibit the phenomena of tautomerism, conformational isomerism, geometric isomerism and/or optical isomerism. It should be understood that the invention encompasses any tautomeric, conformational isomeric, optical isomeric and/or geometric isomeric forms of the MAP kinase and/or HMG-CoA reductase inhibitors described herein, as well as mixtures of these various different forms. For example, optically active compounds of the present invention may be administered in enantiomerically pure (or substantially pure) form or as a mixture of detrorotatory and levorotatory enantiomers, such as in a racemic mixture. It will also be appreciated that compounds disclosed herein can exist in different crystalline forms, including, e.g., polymorphs. The invention encompasses these different crystalline forms, mixtures of different crystalline forms, and pure or substantially pure crystalline forms.

The compounds disclosed in this invention can be produced by methods known in the art as they are derivatives of classes of compounds known in the art.

The present invention relates to these compounds, to pharmaceutical formulations comprising one of more of these compounds, e.g., in combination formulations, and to the use of such compounds and/or the corresponding acids in treating MAP kinase-related and/or HMG-CoA reductase-related conditions, as described in more detail below.

The compounds disclosed in this invention can be produced by methods known in the art as they are derivatives of classes of compounds known in the art. For example, the synthesis of statins is described in Roth et al., J. Med. Chem., 34:357-366 (1991); Krause et al., J. Drug Dev., 3(Suppl. 1):255-257 (1990); and Karanewsky, et al., J. Med. Chem. 33:2952-2956 (1990).

In certain embodiments, the compounds of the present invention can be made using commercially available compounds as starting materials. For example, lactone forms can be prepared from commercially available salts of HMG-CoA reductase inhibitors. For instance, commercially available calcium or sodium salts of atorvastatin, fluvastatin and rosuvastatin may be converted to their protonated free acid forms by extracting the salt forms from weakly acidic aqueous media into an aprotic organic solvent such as ethyl acetate. By stirring the free acid forms in this or another aprotic organic solvent (such as toluene) approximately at or above room temperature, spontaneous conversion to the lactone form occurs over a timeframe of about hours to about days. The lactone forms may be conveniently purified by any methods known in the art, including by column, preparative thin-layer, rotating, or high-pressure chromatography on silica gel columns using standard eluting solvent systems such as about 5:1 (v:v) acetone:ethyl acetate.

In other embodiments, compounds of the present invention can be made from modifying intermediates of synthesis pathways of known statins. For example, a group can be replaced by reactive groups such as an amino, halogen, or hydroxy group, or a metal derivative such as sodium, magnesium, or lithium, and these groups further reacted. Further, those skilled in art will recognize that compounds of the present invention synthesized by various art-known methods will give cis/trans isomers, E/Z forms, diastereomers, and optical isomers, all of which are included in the present invention.

Another aspect of the present invention relates to analogs of known lipophilic MAP kinase and/or HMG-CoA reductase inhibitors, e.g. statins, having structures modified to favor and/or enforce a closed ring structure, for example, a ring structure or cyclic form that is not hydrolyzed or not substantially hydrolyzed to its carboxylic acid or carboxylate forms. “Not hydrolyzed” and “not substantially hydrolyzed,” along with their grammatical conjugations, include situations where some of the compound is hydrolyzed while some is not hydrolyzed. Preferably, at least about 50%, at least about 75%, at least about 90%, and more preferably at least about 95% of the compound is in a ring structure of cyclic form at equilibrium, in situations where the compound is not substantially hydrolyzed. Preferably, at least about 70%, at least about 80%, at least about 90%, and more preferably at least about 95%, and even more preferably at least about 98% of the compound is in a ring structure or cyclic form at equilibrium, in situations where the compound is not hydrolyzed.

In choosing compounds of the present invention, one of ordinary skill in the art will recognize that the various substituents, i.e. R₁, R₂, etc., are to be chosen in conformity with well-known principles of chemical structure connectivity.

The term “substituted” can include multiple degrees of substitution by a named substitutent. Where multiple substituent moieties are disclosed or claimed, the substituted compound can be independently substituted with one or more of the disclosed or claimed substituent moieties, singly or pluraly.

“Alkyl”, as well as other groups having the prefix “alk”, such as alkoxy, alkanoyl, can refer to optionally substituted carbon chains which may be linear or branched or combinations thereof. Examples of alkyl groups include, e.g., methyl, ethyl, n-propyl, isopropyl, n-butyl, iso- sec- and tert-butyl, pentyl, hexyl, heptyl, octyl, nonyl, and the like.

“Cycloalkyl” can refer to optionally substituted carbon rings comprising from 3 to 8 members with 0-2 sites of unsaturation, e.g., cyclopropyl, cyclobutyl, cycloopentyl, cyclohexyl, cycloheptanyl, cyclooctanyl, cyclopenten-3-yl, cyclohexen-1-yl, 1,4-cyclooctadienyl, and the like. In preferred embodiments, the cycloalkyl groups comprise 3-, 4-, or 5-membered rings.

“Aryl” can refer to optionally substituted mono- or bicyclic aromatic rings containing only carbon atoms. The term can also include aryl group fused to a monocyclic cycloalkyl or monocyclic cycloheteroalkyl group in which the point of attachment is on an aromatic portion. Examples of aryl groups include, e.g., phenyl, naphthyl, indanyl, indenyl, tetrahydronaphthyl, 2,3-dihydrobenzofuranyl, dihydrobenzopyranyl, 1,4-benzodioxanyl, and the like.

“Heteroaryl” can refer to an optionally substituted mono- or bicyclic aromatic ring containing at least one heteroatom (an atom other than carbon), such as N, O and S, with each ring containing about 5 to about 6 atoms. Examples of heteroaryl groups include, e.g., pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Halogen” can include fluorine, chlorine, bromine and iodine.

As used herein, R, R′, etc., generally refer to any non-aromatic group, including, e.g., substituted or unsubstituted alkyl groups, unless specifically defined otherwise. Ar, Ar′, etc., generally refer to substituted or unsubstituted aromatic groups, including, e.g., aryls and heteroaryls.

In some preferred embodiments, n is 0 1, 2 or 3. In some preferred embodiments, R₁ has the following stereochemistry:

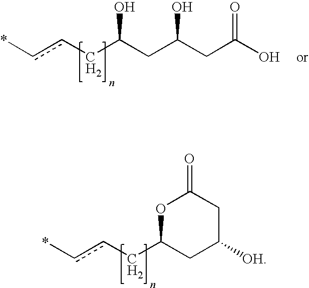

Some of the compounds described herein contain olefinic double bonds, and unless specified otherwise, are meant to include both E and/or Z geometric isomers. In some embodiments, the E geometric isomer is preferred.

Some of the compounds described herein may exist with different points of attachment of hydrogen, referred to as tautomers. Such an example may be a ketone and its enol form known as keto-enol tautomers.

Compounds of the present invention may be separated into diastereoisomeric pairs of enantiomers by, for example, fractional crystallization from a suitable solvent, for example MeOH or ethyl acetate or a mixture thereof. The pair of enantiomers may be separated into individual stereoisomers by, for example the use of an optically active amine as a resolving agent or on a chiral HPLC column. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

Alternatively, any enantiomer of a compound may be obtained by stereospecific synthesis using optically pure starting materials or reagents of known configuration. In preferred embodiments, compounds of the present invention are administered as enantiomerically pure (or substantially enantiomerically pure) formulations.

For example, depicted below are synthesis schemes for compounds of Formula XXXII. The general Synthetic Scheme 1, preferred for compounds wherein R₁ is saturated; i.e., does not comprise a carbon-carbon double bond is as follows:

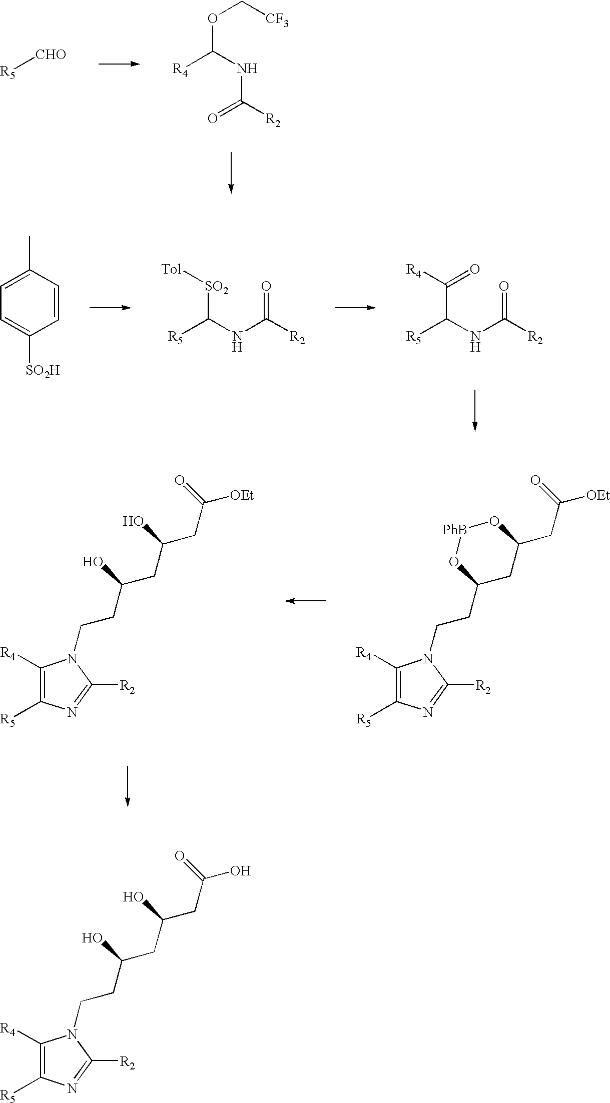

The general synthetic scheme 2, preferred for compounds wherein R₁ is unsaturated; i.e., comprises a carbon-carbon double bond is as follows:

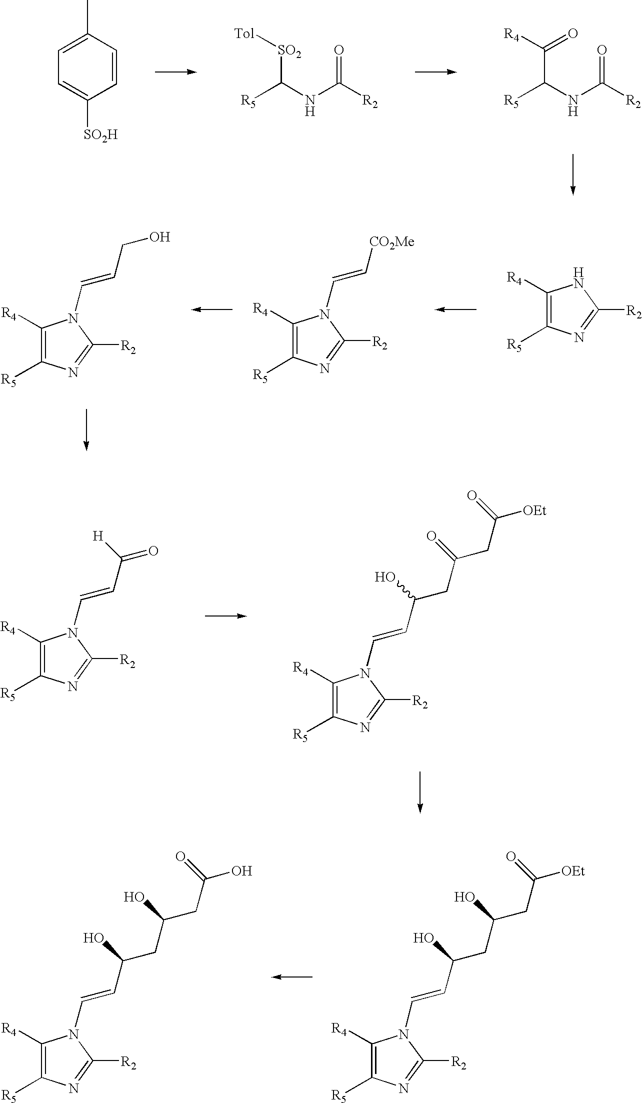

### II. Compounds of the Invention

The present invention includes the compounds disclosed used herein and methods of use thereof. Preferably, the compounds are used in methods for inhibition of MAP kinase. The present invention encompasses the compounds disclosed herein and pharmaceutically acceptable salts thereof. In the compounds disclosed herein, n is preferably 0 or 2. In preferred embodiments, n is one, greater than 2, greater than 5, greater than 7, greater, than 10, greater than 12, greater than 15, or greater than 17. Preferably, n is less than 20, less than 17, less than 15, less than 12, less than 10, less than 7, or less than 5. Preferably, n ranges from 0-2, 3-5, 6-8, 9-10, 11-14, 15-20, 21-25, or 25-30. M⁺, as depicted in certain compounds, is a cation, preferably a sodium or calcium ion.

One aspect of the present invention provides compositions and methods of inhibiting a MAP kinase comprising administering an effective amount of at least one compound comprising formula XX:

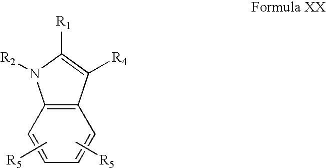

wherein R₁ is

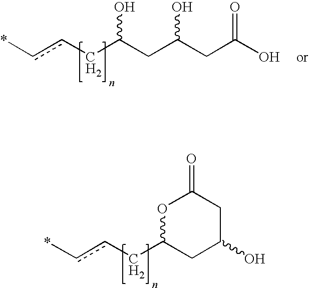

n being 0 or any integer;

R₂ is optionally substituted alkyl, cycloalkyl, aryl, or heteroaryl;

R₄ is optionally substituted

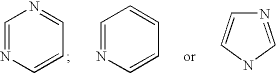

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula XX:

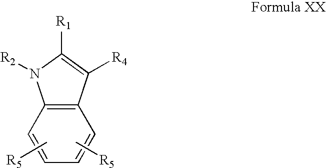

wherein R₁ is

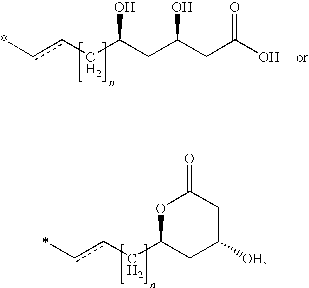

n being 0 or any integer;

R₂ is optionally substituted alkyl, cycloalkyl, aryl, or heteroaryl;

R₄ is optionally substituted

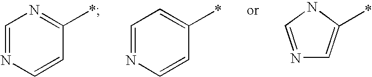

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides compositions and methods of inhibiting a MAP kinase comprising administering an effective amount of at least one compound comprising formula XXI:

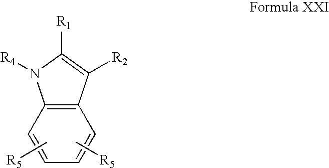

wherein R₁ is

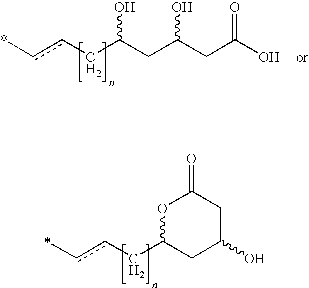

n being 0 or any integer;

R₂ is optionally substituted alkyl, cycloalkyl, aryl, or heteroaryl;

R₄ is optionally substituted

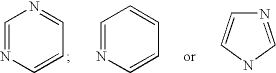

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula XXI:

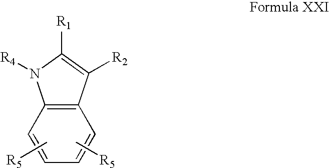

wherein R₁ is

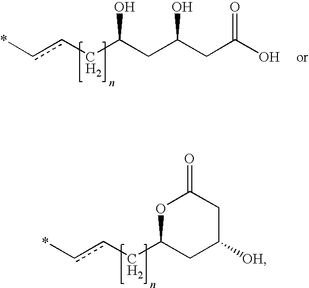

n being 0 or any integer;

R₂ is optionally substituted alkyl, cycloalkyl, aryl, or heteroaryl;

R₄ is optionally substituted

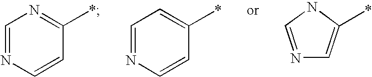

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides compositions and methods of inhibiting a MAP kinase comprising administering an effective amount of at least one compound comprising formula XXII:

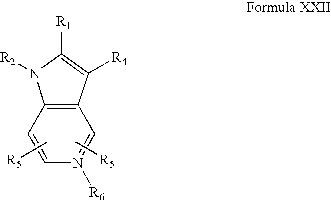

wherein R₁ is

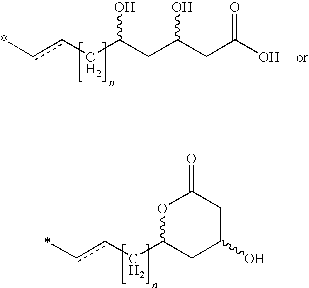

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXII:

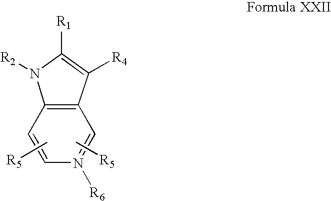

wherein R₁ is

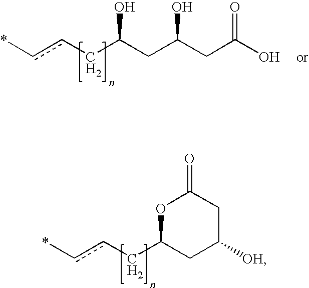

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXIII:

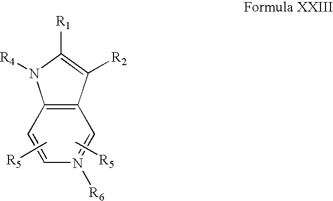

wherein R₁ is

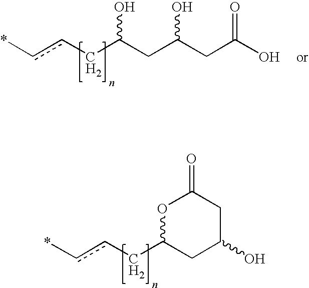

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXIII:

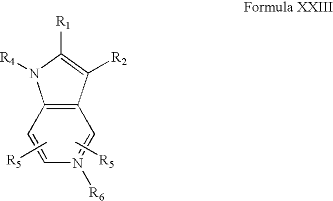

wherein R₁ is

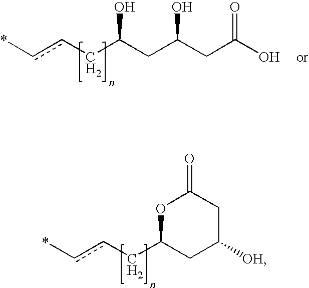

n being 0 or any integer;

R₂ is optionally substituted alkyl, cycloalkyl, aryl, or heteroaryl;

R₄ is optionally substituted alkyl, cycloalkyl, aryl, or heteroaryl

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXIV:

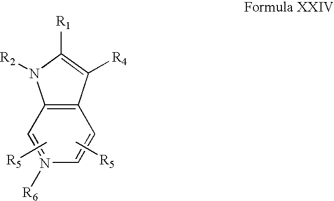

wherein R₁ is

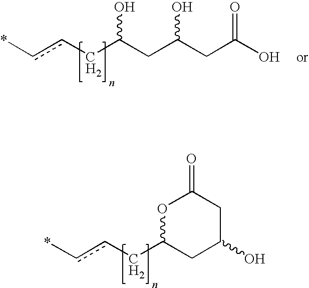

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXIV:

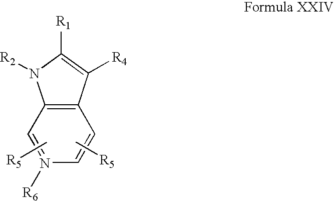

wherein R₁ is

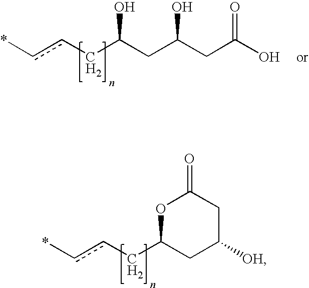

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXV:

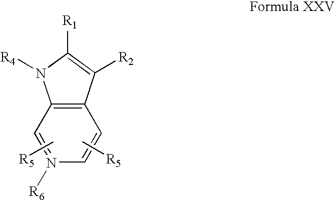

wherein R₁ is

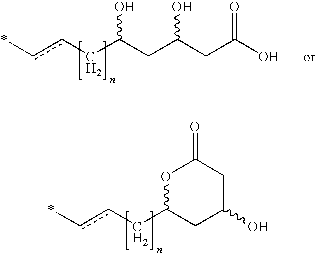

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₅ is either a lone pair of electrons or an oxygen atom.

Another aspect of the present invention provides a compound comprising formula XXV:

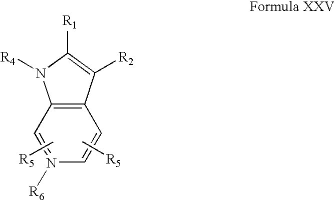

wherein R₁ is

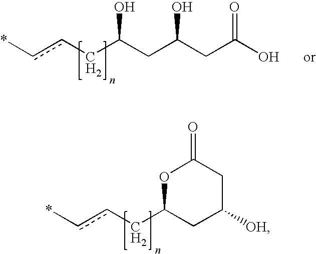

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXVI:

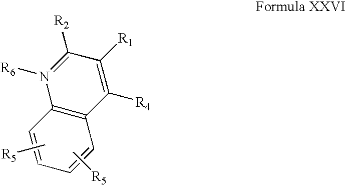

wherein R₁ is

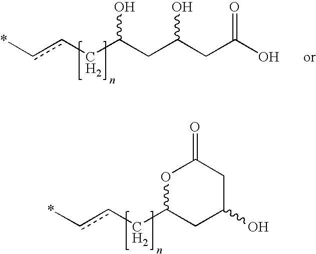

n being 0 or any integer;

R₂ is optionally substituted alkyl, cycloalkyl, aryl, or heteroaryl;

R₄ is optionally substituted

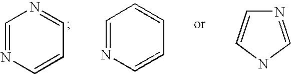

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

Another aspect of the present invention provides a compound comprising formula XXVI:

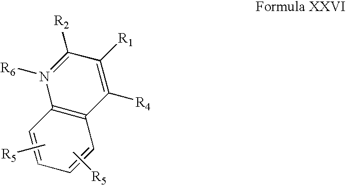

wherein R₁ is

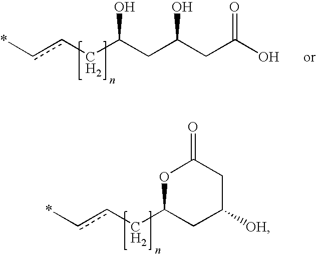

n being 0 or any integer;

R₂ is optionally substituted alkyl, cycloalkyl, aryl, or heteroaryl;

R₄ is optionally substituted

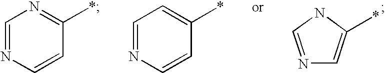

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

One aspect of the present invention provides compounds and methods of inhibiting a MAP kinase comprising administering an effective amount of at least one compound comprising formula XXVII:

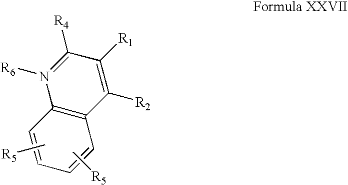

wherein R₁ is

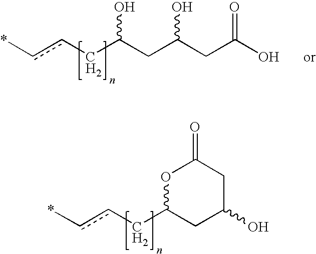

n being 0 or any integer;

R₂ is optionally substituted alkyl, cycloalkyl, aryl, or heteroaryl;

R₄ is optionally substituted

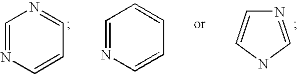

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

Another aspect of the present invention provides a compound comprising formula XXVII:

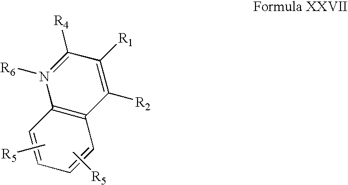

wherein R₁ is

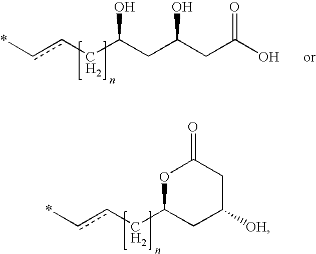

n being 0 or any integer;

R₂ is optionally substituted alkyl, cycloalkyl, aryl, or heteroaryl;

R₄ is optionally substituted

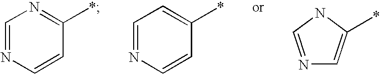

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair or an oxygen atom.

One aspect of the present invention provides compounds and methods of inhibiting a MAP kinase comprising administering an effective amount of at least one compound comprising formula XXVIII:

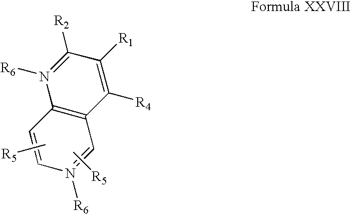

wherein R₁ is

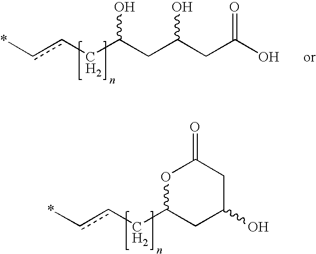

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXVIII:

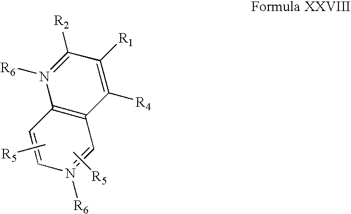

wherein R₁ is

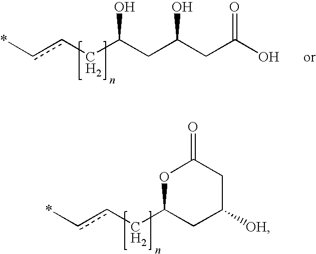

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, optionally substituted amino; and

R₆ is either a lone pair of electrons or an oxygen atom.

One aspect of the present invention provides compounds and methods of inhibiting a MAP kinase comprising administering an effective amount of at least one compound comprising formula XXIX:

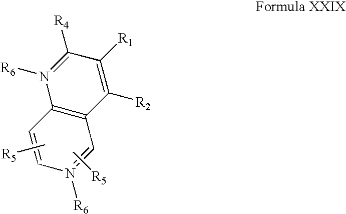

wherein R₁ is

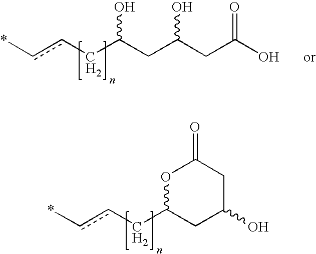

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXIX:

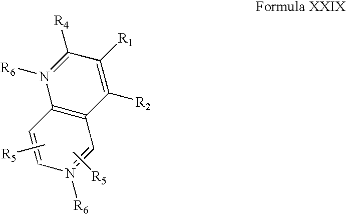

wherein R₁ is

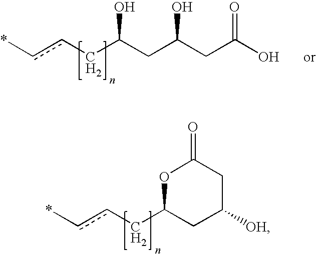

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXX:

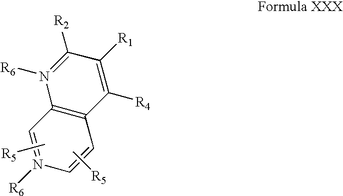

wherein R₁ is

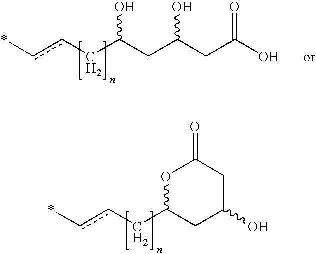

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXX:

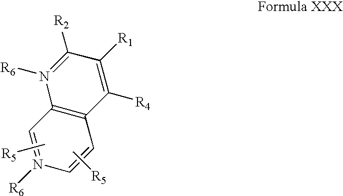

wherein R₁ is

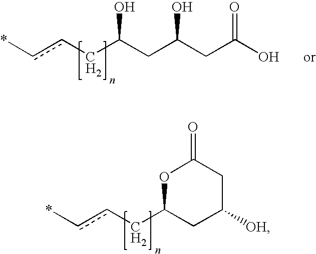

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides compounds and methods of inhibiting a MAP kinase comprising administering an effective amount of at least one compound comprising formula XXXI:

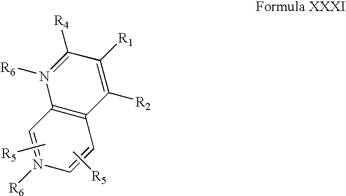

wherein R₁ is

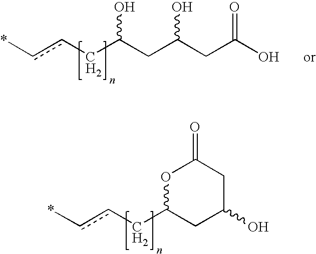

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula XXXI:

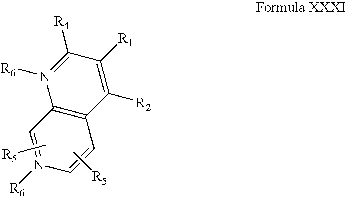

wherein R₁ is

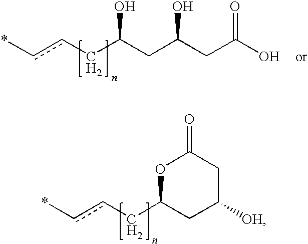

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Some embodiments provide a compound comprising formula XXXII:

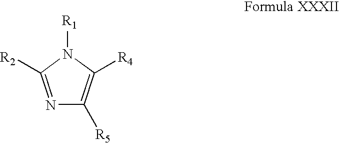

wherein R₁ is

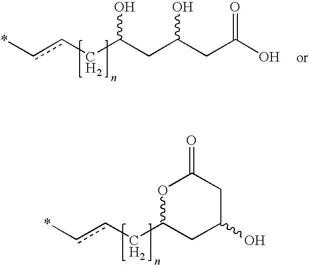

n being 0 or any integer;

R₂ is optionally substituted alkyl, cycloalkyl, aryl, or heteroaryl;

R₄ is optionally substituted

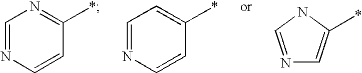

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXII:

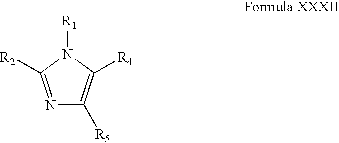

wherein R₁ is

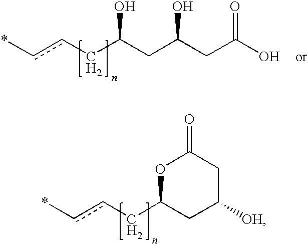

n being 0 or any integer;

R₂ is optionally substituted alkyl, cycloalkyl, aryl, or heteroaryl;

R₄ is optionally substituted

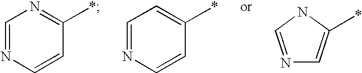

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXIII:

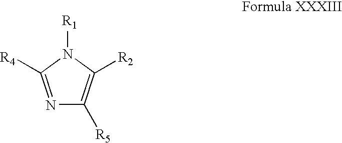

wherein R₁ is

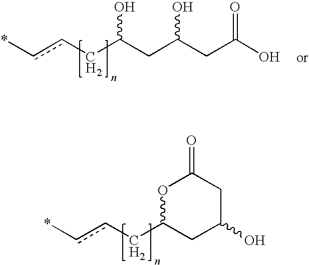

n being 0 or any integer;

R₂ is optionally substituted alkyl, cycloalkyl, aryl, or heteroaryl;

R₄ is optionally substituted

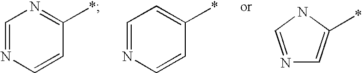

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXIII:

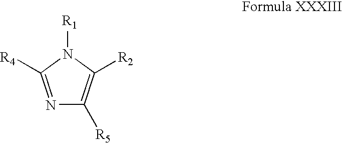

wherein R₁ is

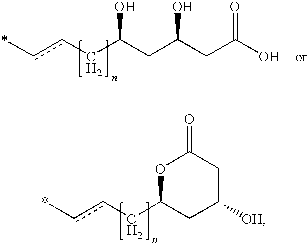

n being 0 or any integer;

R₂ is optionally substituted alkyl, cycloalkyl, aryl, or heteroaryl;

R₄ is optionally substituted

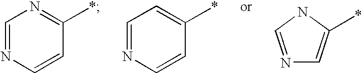

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXIV:

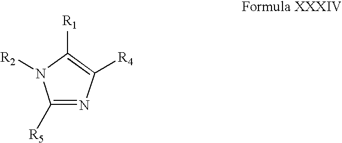

wherein R₁ is

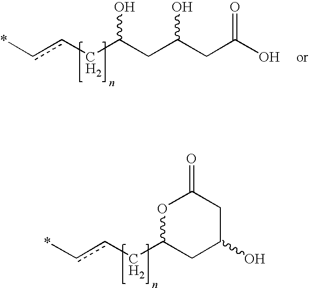

n being 0 or any integer;

R₂ is optionally substituted alkyl, cycloalkyl, aryl, or heteroaryl;

R₄ is optionally substituted

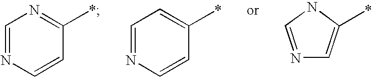

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXIV:

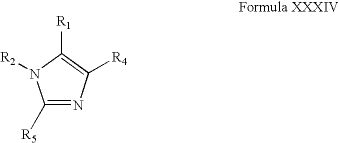

wherein R₁ is

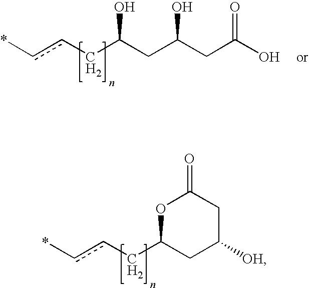

n being 0 or any integer;

R₂ is optionally substituted alkyl, cycloalkyl, aryl, or heteroaryl;

R₄ is optionally substituted

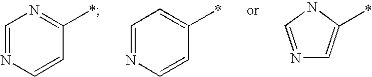

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXV:

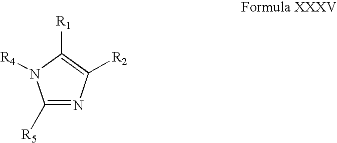

wherein R₁ is

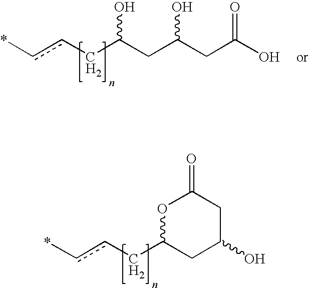

n being 0 or any integer;

R₂ is optionally substituted alkyl, cycloalkyl, aryl, or heteroaryl;

R₄ is optionally substituted

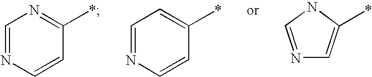

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXV:

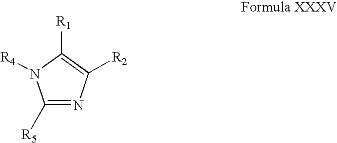

wherein R₁ is

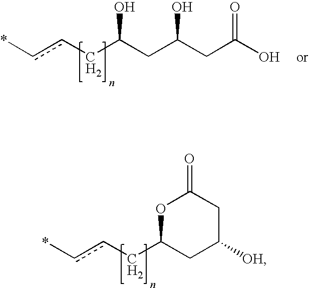

n being 0 or any integer;

R₂ is optionally substituted alkyl, cycloalkyl, aryl, or heteroaryl;

R₄ is optionally substituted

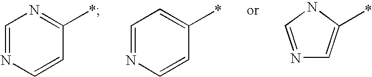

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXVI:

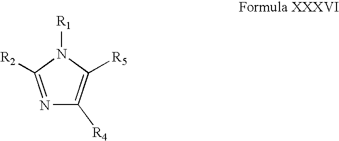

wherein R₁ is

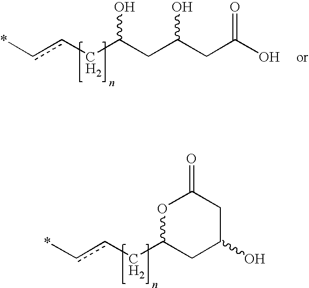

n being 0 or any integer;

R₂ is optionally substituted alkyl, cycloalkyl, aryl, or heteroaryl;

R₄ is optionally substituted

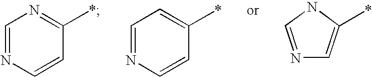

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXVI:

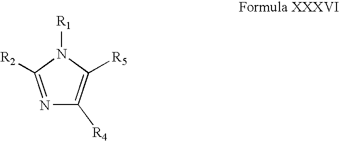

wherein R₁ is

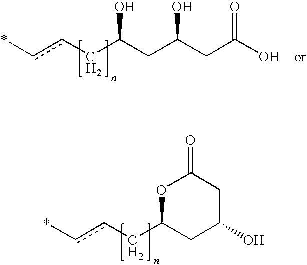

n being 0 or any integer;

R₂ is optionally substituted alkyl, cycloalkyl, aryl, or heteroaryl;

R₄ is optionally substituted

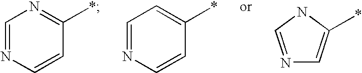

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXVII:

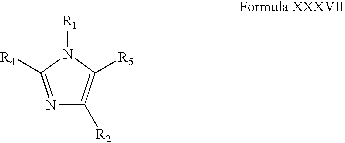

wherein R₁ is

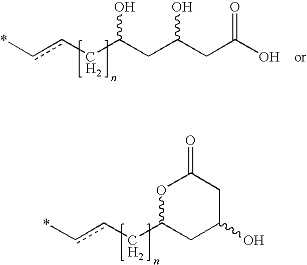

n being 0 or any integer;

R₂ is optionally substituted alkyl, cycloalkyl, aryl, or heteroaryl;

R₄ is optionally substituted

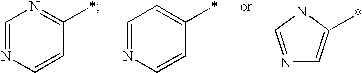

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXVII:

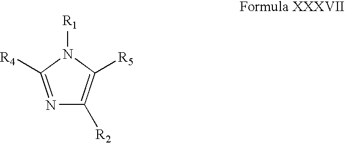

wherein R₁ is

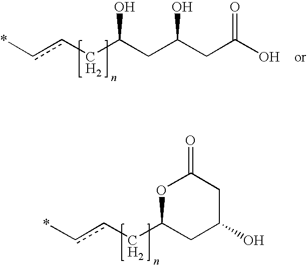

n being 0 or any integer;

R₂ is optionally substituted alkyl, cycloalkyl, aryl, or heteroaryl;

R₄ is optionally substituted

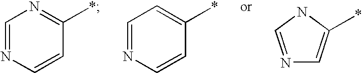

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXVIII:

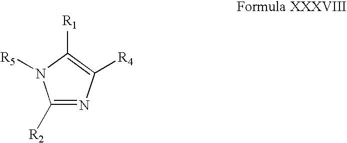

wherein R₁ is

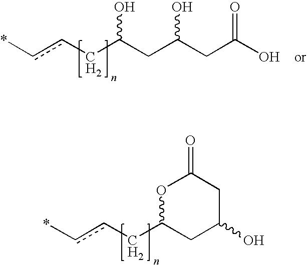

n being 0 or any integer;

R₂ is optionally substituted alkyl, cycloalkyl, aryl, or heteroaryl;

R₄ is optionally substituted

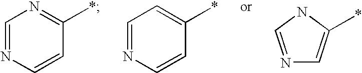

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXVIII:

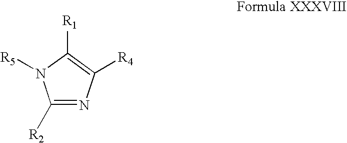

wherein R₁ is

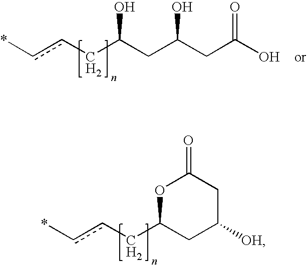

n being 0 or any integer;

R₂ is optionally substituted alkyl, cycloalkyl, aryl, or heteroaryl;

R₄ is optionally substituted

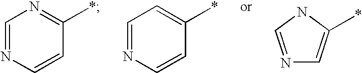

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXIX:

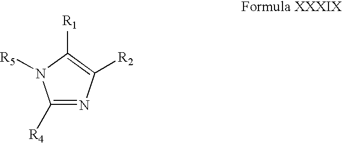

wherein R₁ is

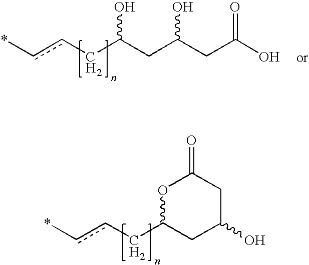

n being 0 or any integer;

R₂ is optionally substituted alkyl, cycloalkyl, aryl, or heteroaryl;

R₄ is optionally substituted

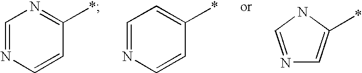

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXIX:

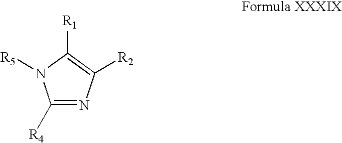

wherein R₁ is

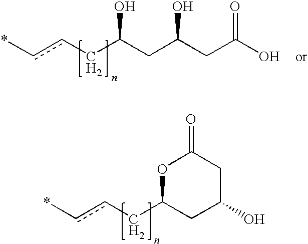

n being 0 or any integer;

R₂ is optionally substituted alkyl, cycloalkyl, aryl, or heteroaryl;

R₄ is optionally substituted

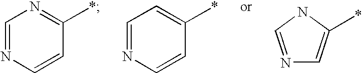

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXX:

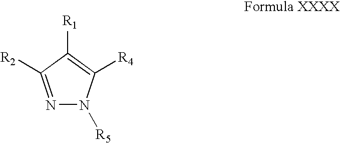

wherein R₁ is

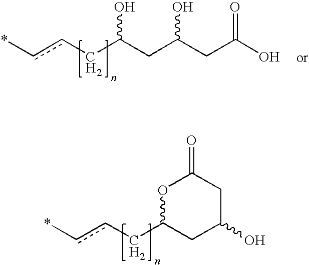

n being 0 or any integer;

R₂ is optionally substituted alkyl, cycloalkyl, aryl, or heteroaryl;

R₄ is optionally substituted

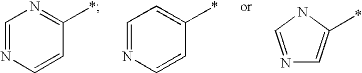

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXX:

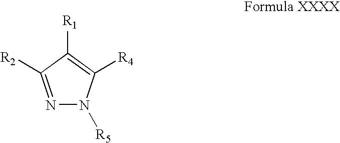

wherein R₁ is

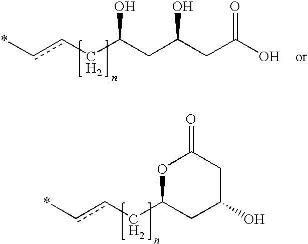

n being 0 or any integer;

R₂ is optionally substituted alkyl, cycloalkyl, aryl, or heteroaryl;

R₄ is optionally substituted

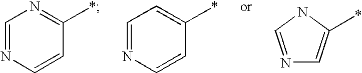

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXI:

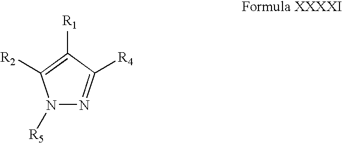

wherein R₁ is

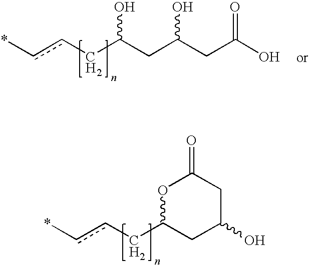

n being 0 or any integer;

R₂ is optionally substituted alkyl, cycloalkyl, aryl, or heteroaryl;

R₄ is optionally substituted

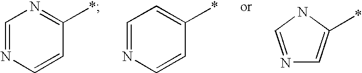

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXI:

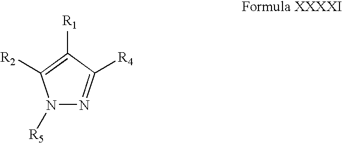

wherein R₁ is

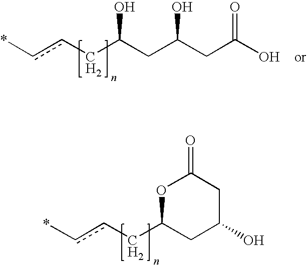

n being 0 or any integer;

R₂ is optionally substituted alkyl, cycloalkyl, aryl, or heteroaryl;

R₄ is optionally substituted

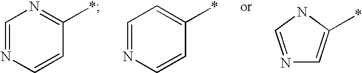

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXII:

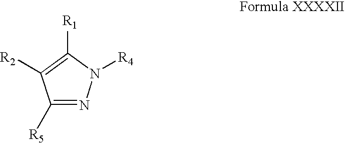

wherein R₁ is

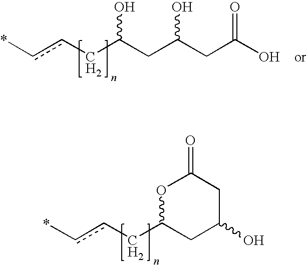

n being 0 or any integer;

R₂ is optionally substituted alkyl, cycloalkyl, aryl, or heteroaryl;

R₄ is optionally substituted

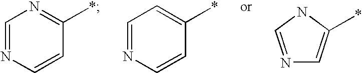

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXII:

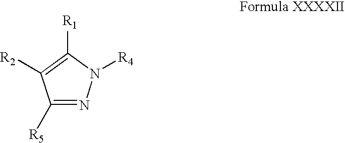

wherein R₁ is

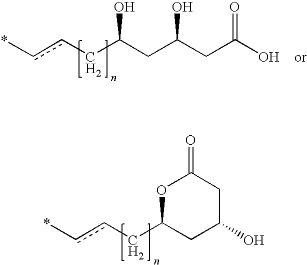

n being 0 or any integer;

R₂ is optionally substituted alkyl, cycloalkyl, aryl, or heteroaryl;

R₄ is optionally substituted

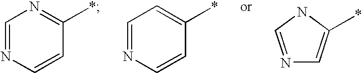

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXIII:

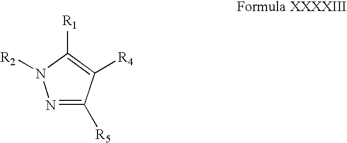

wherein R₁ is

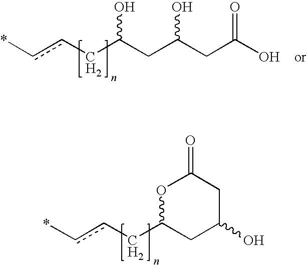

n being 0 or any integer;

R₂ is optionally substituted alkyl, cycloalkyl, aryl, or heteroaryl;

R₄ is optionally substituted

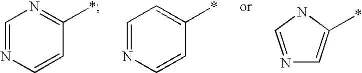

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXIII:

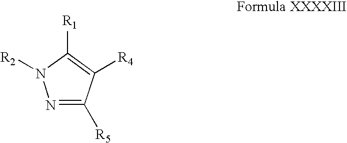

wherein R₁ is

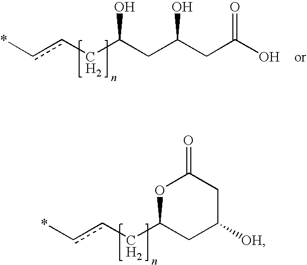

n being 0 or any integer;

R₂ is optionally substituted alkyl, cycloalkyl, aryl, or heteroaryl;

R₄ is optionally substituted

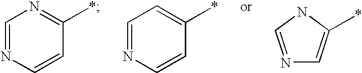

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXIV:

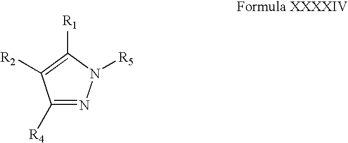

wherein R₁ is

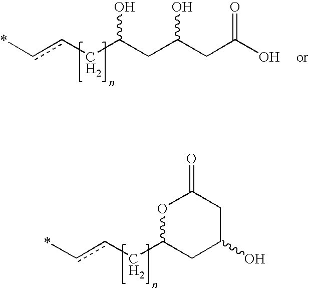

n being 0 or any integer;

R₂ is optionally substituted alkyl, cycloalkyl, aryl, or heteroaryl;

R₄ is optionally substituted

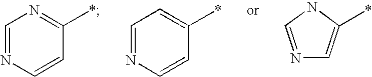

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXIV:

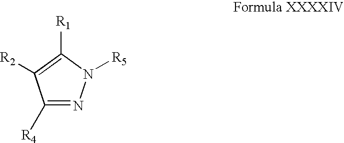

wherein R₁ is

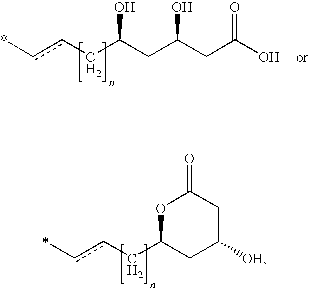

n being 0 or any integer;

R₂ is optionally substituted alkyl, cycloalkyl, aryl, or heteroaryl;

R₄ is optionally substituted

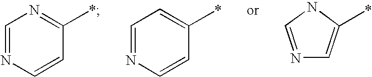

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXV:

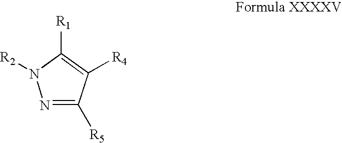

wherein R₁ is

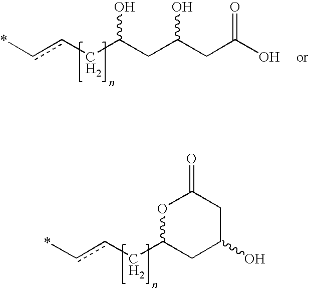

n being 0 or any integer;

R₂ is optionally substituted alkyl, cycloalkyl, aryl, or heteroaryl;

R₄ is optionally substituted

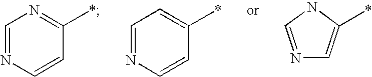

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXV:

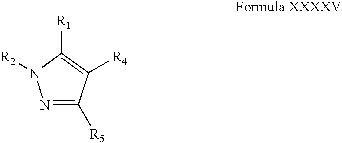

wherein R₁ is

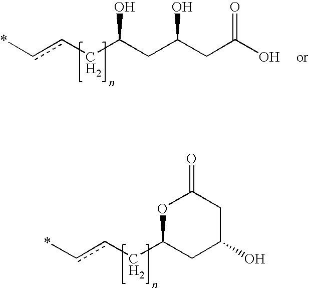

n being 0 or any integer;

R₂ is optionally substituted alkyl, cycloalkyl, aryl, or heteroaryl;

R₄ is optionally substituted

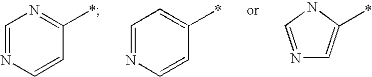

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXVI:

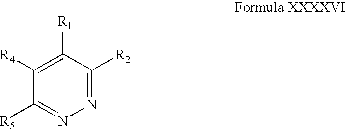

wherein R₁ is

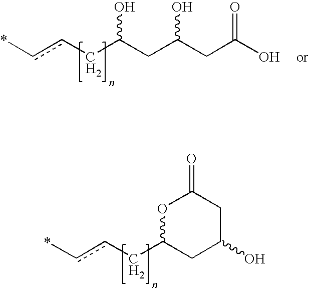

n being 0 or any integer;

R₂ is optionally substituted alkyl, cycloalkyl, aryl, or heteroaryl;

R₄ is optionally substituted

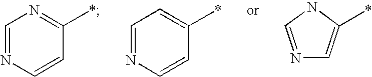

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXVI:

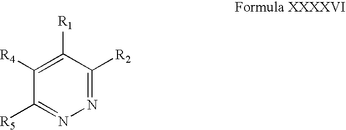

wherein R₁ is

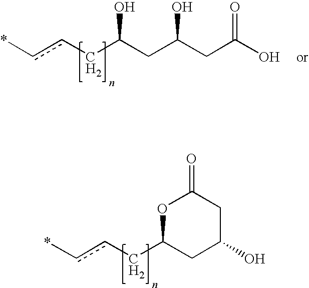

n being 0 or any integer;

R₂ is optionally substituted alkyl, cycloalkyl, aryl, or heteroaryl;

R₄ is optionally substituted

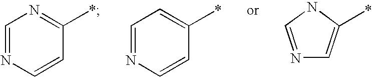

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXVII:

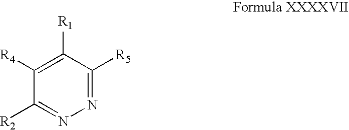

wherein R₁ is

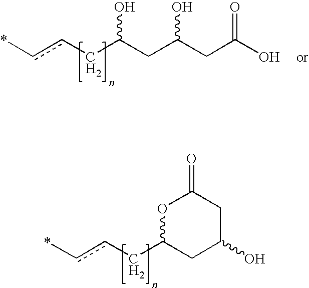

n being 0 or any integer;

R₂ is optionally substituted alkyl, cycloalkyl, aryl, or heteroaryl;

R₄ is optionally substituted

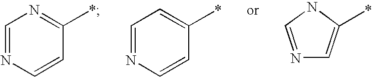

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXVII:

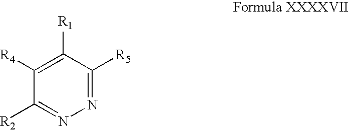

wherein R₁ is

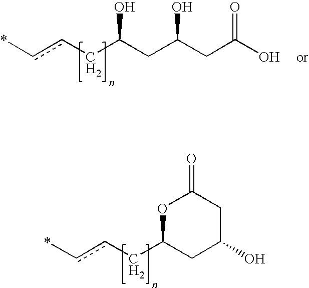

n being 0 or any integer;

R₂ is optionally substituted alkyl, cycloalkyl, aryl, or heteroaryl;

R₄ is optionally substituted

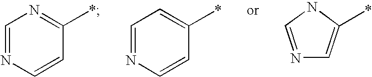

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXVIII:

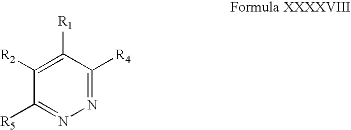

wherein R₁ is

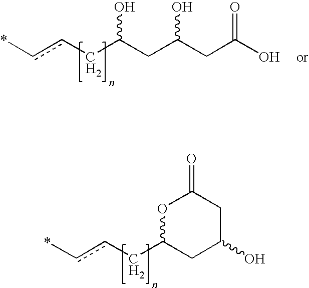

n being 0 or any integer;

R₂ is optionally substituted alkyl, cycloalkyl, aryl, or heteroaryl;

R₄ is optionally substituted

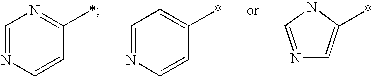

and R₁ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXVIII:

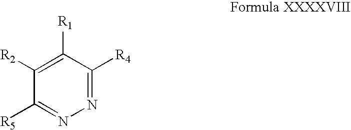

wherein R₁ is

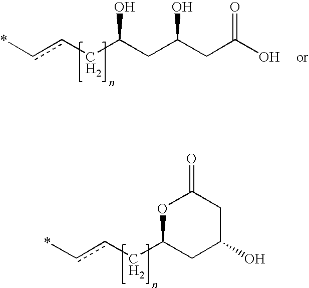

n being 0 or any integer;

R₂ is optionally substituted alkyl, cycloalkyl, aryl, or heteroaryl;

R₄ is optionally substituted

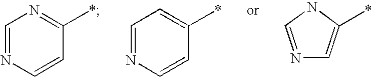

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula XXXXIX:

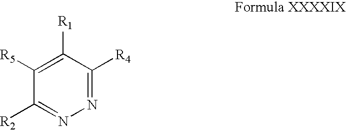

wherein R₁ is

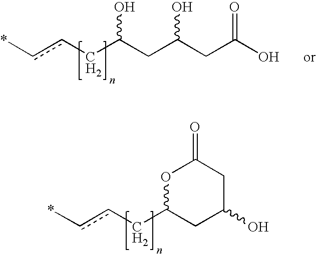

n being 0 or any integer;

R₂ is optionally substituted alkyl, cycloalkyl, aryl, or heteroaryl;

R₄ is optionally substituted

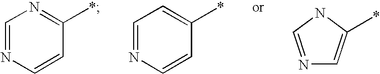

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula XXXXIX:

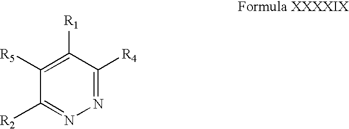

wherein R₁ is

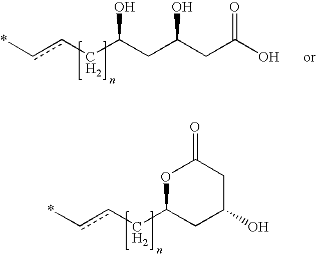

n being 0 or any integer;

R₂ is optionally substituted alkyl, cycloalkyl, aryl, or heteroaryl;

R₄ is optionally substituted

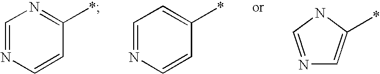

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula L:

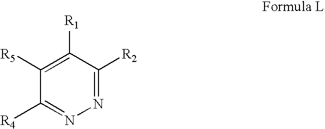

wherein R₁ is

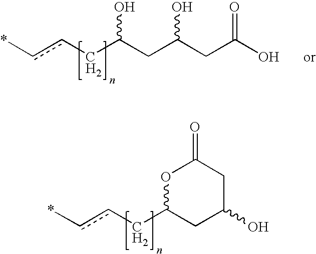

n being 0 or any integer;

R₂ is optionally substituted alkyl, cycloalkyl, aryl, or heteroaryl;

R₄ is optionally substituted

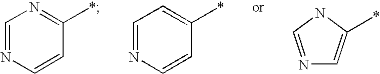

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula L:

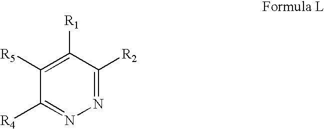

wherein R₁ is

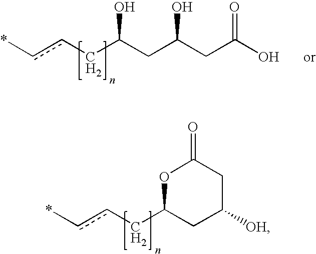

n being 0 or any integer;

R₂ is optionally substituted alkyl, cycloalkyl, aryl, or heteroaryl;

R₄ is optionally substituted

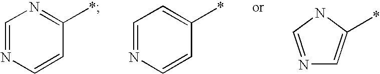

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LI:

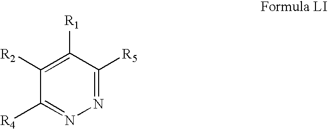

wherein R₁ is

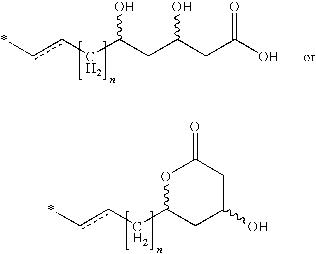

n being 0 or any integer;

R₂ is optionally substituted alkyl, cycloalkyl, aryl, or heteroaryl;

R₄ is optionally substituted

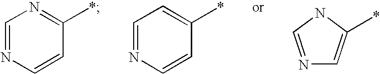

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LI:

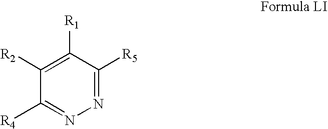

wherein R₁ is

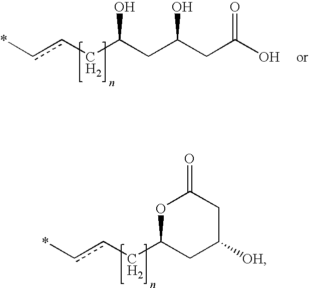

n being 0 or any integer;

R₂ is optionally substituted alkyl, cycloalkyl, aryl, or heteroaryl;

R₄ is optionally substituted

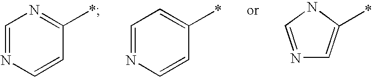

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LII:

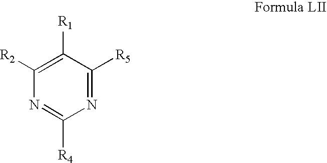

wherein R₁ is

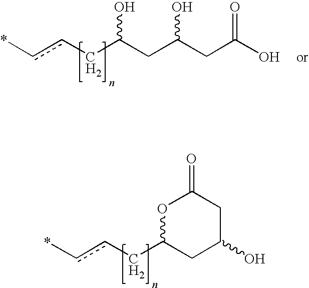

n being 0 or any Integer;

R₂ is optionally substituted alkyl, cycloalkyl, aryl, or heteroaryl;

R₄ is optionally substituted

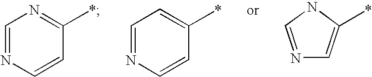

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LII:

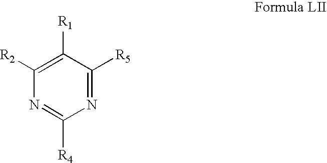

wherein R₁ is

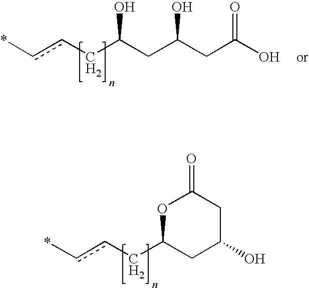

n being 0 or any integer;

R₂ is optionally substituted alkyl, cycloalkyl, aryl, or heteroaryl;

R₄ is optionally substituted

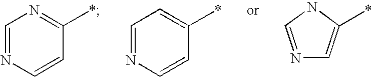

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LIII:

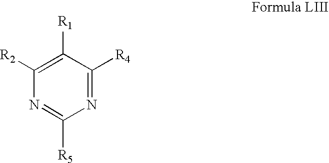

wherein R₁ is

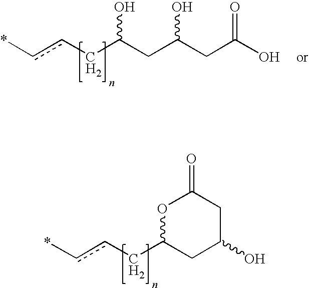

n being 0 or any integer;

R₂ is optionally substituted alkyl, cycloalkyl, aryl, or heteroaryl;

R₄ is optionally substituted

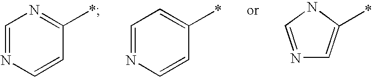

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LIII:

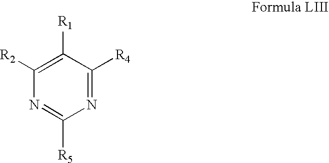

wherein R₁ is

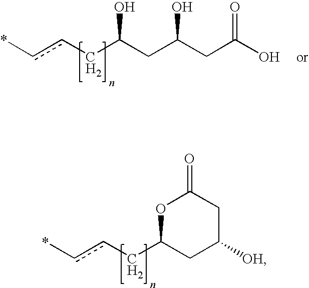

n being 0 or any integer;

R₂ is optionally substituted alkyl, cycloalkyl, aryl, or heteroaryl;

R₄ is optionally substituted

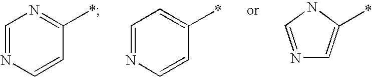

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Some embodiments provide a compound comprising formula LIV:

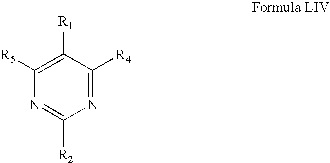

wherein R₁ is

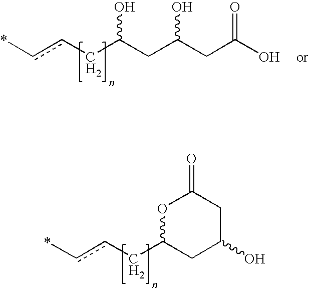

n being 0 or any integer;

R₂ is optionally substituted alkyl, cycloalkyl, aryl, or heteroaryl;

R₄ is optionally substituted

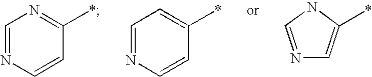

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

Another aspect of the present invention provides a compound comprising formula LIV:

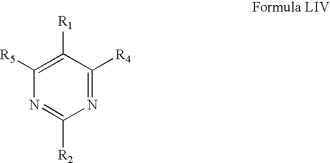

wherein R₁ is

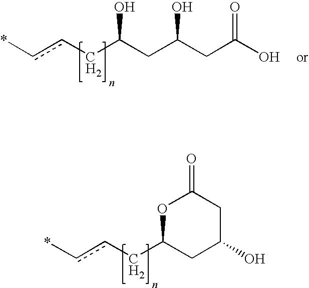

n being 0 or any integer;

R₂ is optionally substituted alkyl, cycloalkyl, aryl, or heteroaryl;

R₄ is optionally substituted

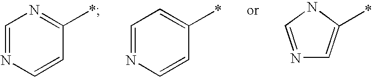

and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a salt thereof.

One aspect of the present invention provides methods of inhibiting a MAP kinase comprising administering an effective amount of at least one compound comprising formula LV:

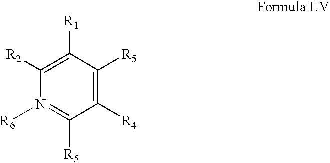

wherein R₁ is

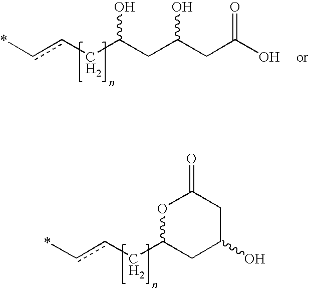

n being 0 or any integer;

R₂ is optionally substituted alkyl, cycloalkyl, aryl, or heteroaryl;

R₄ is optionally substituted

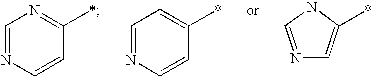

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LV:

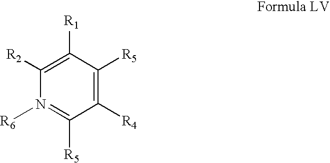

wherein R₁ is

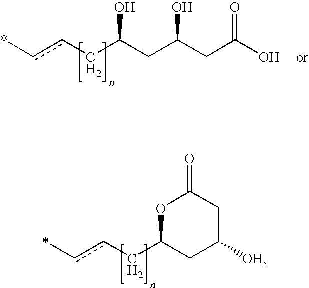

n being 0 or any integer;

R₂ is optionally substituted alkyl, cycloalkyl, aryl, or heteroaryl;

R₄ is optionally substituted

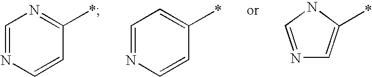

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LVI:

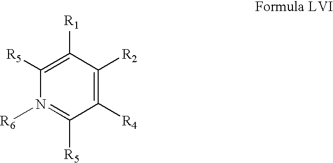

wherein R₁ is

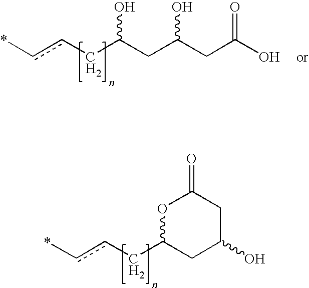

n being 0 or any integer;

R₂ is optionally substituted alkyl, cycloalkyl, aryl, or heteroaryl;

R₄ is optionally substituted

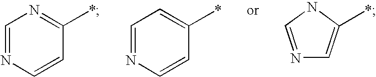

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LVI:

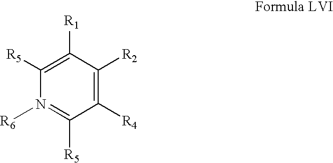

wherein R₁ is

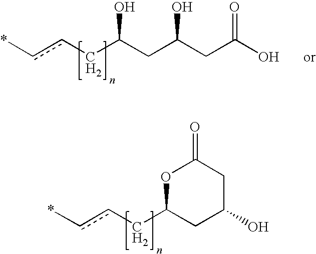

n being 0 or any integer;

R₂ is optionally substituted alkyl, cycloalkyl, aryl, or heteroaryl;

R₄ is optionally substituted

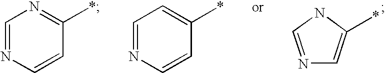

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LVII:

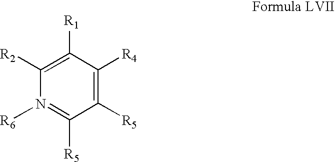

wherein R₁ is

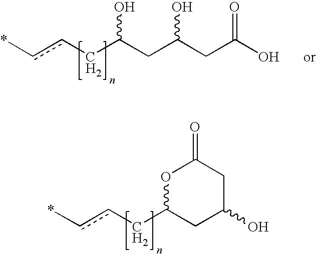

n being 0 or any integer;

R₂ is optionally substituted alkyl, cycloalkyl, aryl, or heteroaryl;

R₄ is optionally substituted

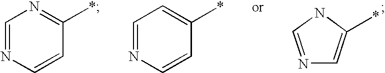

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LVII:

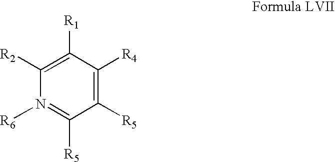

wherein R₁ is

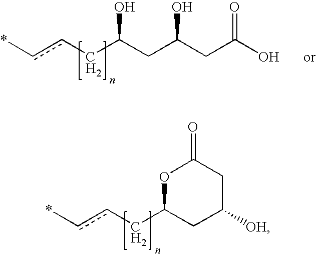

n being 0 or any integer;

R₂ is optionally substituted alkyl, cycloalkyl, aryl, or heteroaryl;

R₄ is optionally substituted

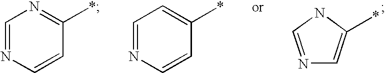

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LVIII:

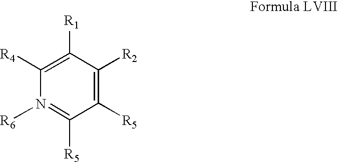

wherein R₁ is

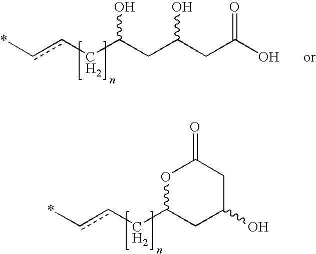

n being 0 or any integer;

R₂ is optionally substituted alkyl, cycloalkyl, aryl, or heteroaryl;

R₄ is optionally substituted

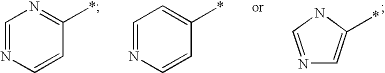

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LVIII:

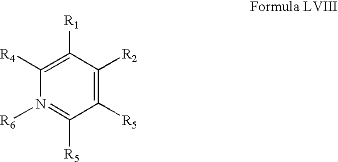

wherein R₁ is

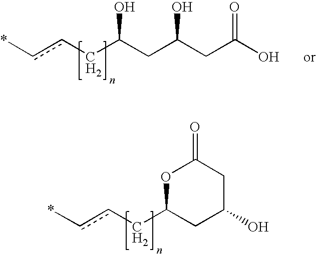

n being 0 or any integer;

R₂ is optionally substituted alkyl, cycloalkyl, aryl, or heteroaryl;

R₄ is optionally substituted

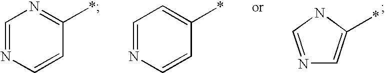

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LIX:

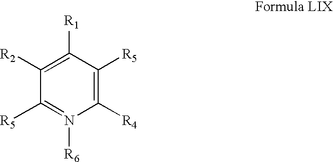

wherein R₁ is

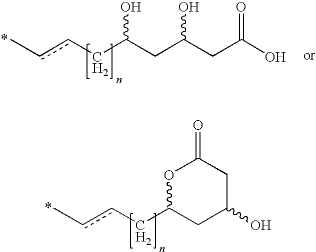

n being 0 or any integer;

R₂ is optionally substituted alkyl, cycloalkyl, aryl, or heteroaryl;

R₄ is optionally substituted

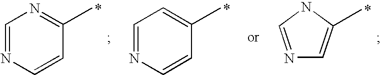

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LIX:

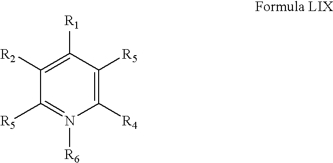

wherein R₁ is

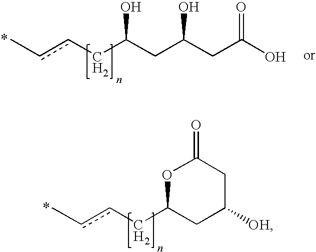

n being 0 or any integer;

R₂ is optionally substituted alkyl, cycloalkyl, aryl, or heteroaryl;

R₄ is optionally substituted

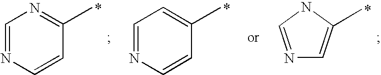

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LX:

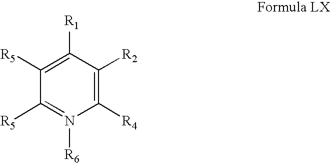

wherein R₁ is

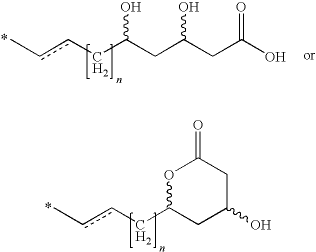

n being 0 or any integer;

R₂ is optionally substituted alkyl, cycloalkyl, aryl, or heteroaryl;

R₄ is optionally substituted

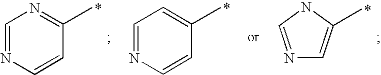

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LX:

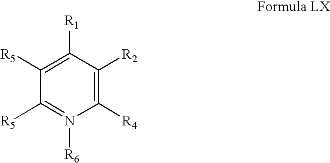

wherein R₁ is

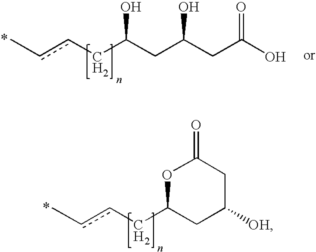

being 0 or any integer;

R₂ is optionally substituted alkyl, cycloalkyl, aryl, or heteroaryl;

R₄ is optionally substituted

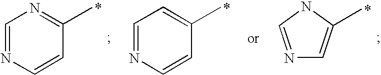

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXI:

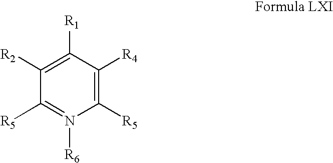

wherein R₁ is

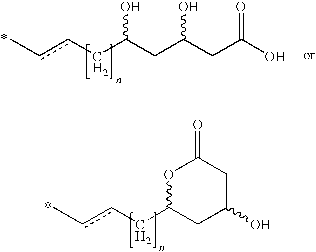

n being 0 or any integer;

R₂ is optionally substituted alkyl, cycloalkyl, aryl, or heteroaryl;

R₄ is optionally substituted

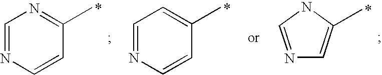

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXI:

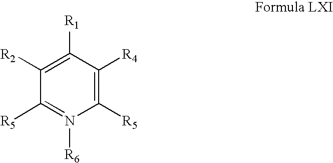

wherein R₁ is

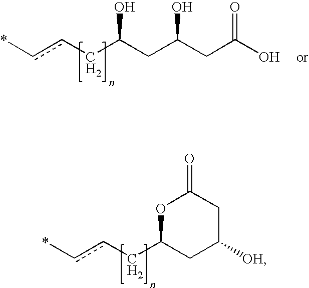

being 0 or any integer;

R₂ is optionally substituted alkyl, cycloalkyl, aryl, or heteroaryl;

R₄ is optionally substituted

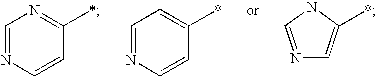

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXII:

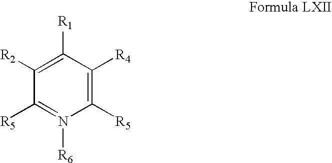

wherein R₁ is

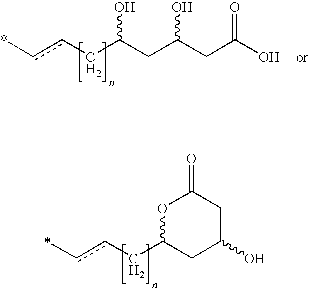

n being 0 or any integer;

R₂ is optionally substituted alkyl, cycloalkyl, aryl, or heteroaryl;

R₄ is optionally substituted

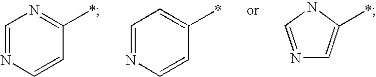

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXII:

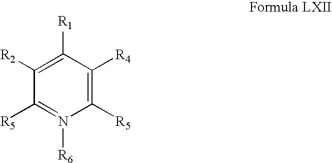

wherein R₁ is

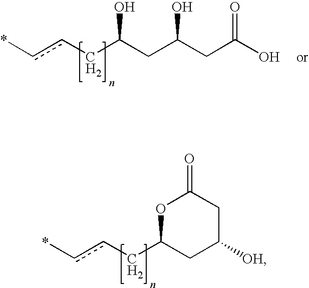

n being 0 or any integer;

R₂ is optionally substituted alkyl, cycloalkyl, aryl, or heteroaryl;

R₄ is optionally substituted

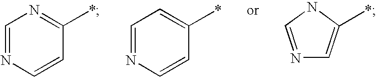

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXIII:

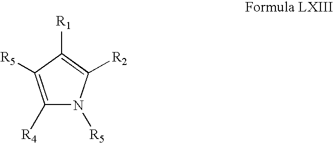

wherein R₁ is

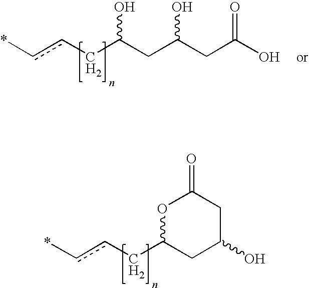

n being 0 or any integer;

R₂ is optionally substituted alkyl, cycloalkyl, aryl, or heteroaryl;

R₄ is optionally substituted

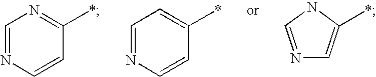

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXIII:

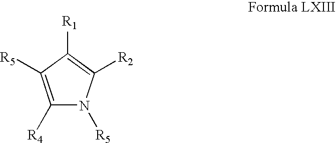

wherein R₁ is

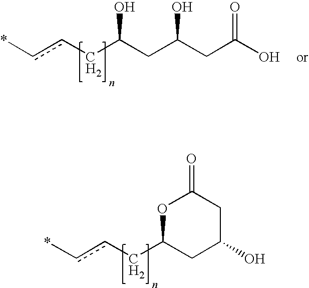

n being 0 or any integer;

R₂ is optionally substituted alkyl, cycloalkyl, aryl, or heteroaryl;

R₄ is optionally substituted

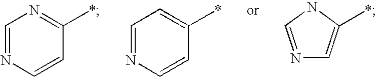

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXIV:

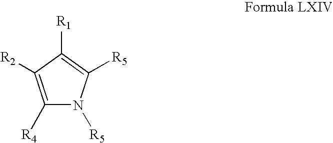

wherein R₁ is

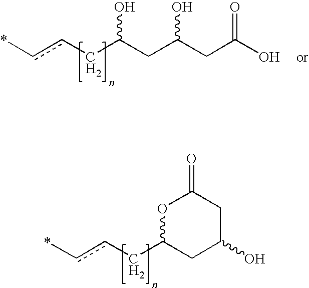

n being 0 or any integer;

R₂ is optionally substituted alkyl, cycloalkyl, aryl, or heteroaryl;

R₄ is optionally substituted

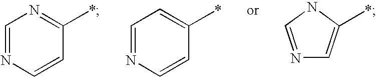

and R₅ is in each instance hydrogen, optionally substituted lower alkyl; optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXIV:

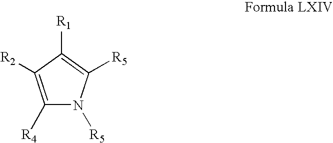

wherein R₁ is

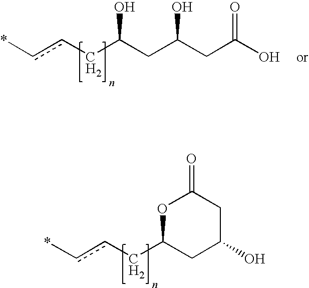

n being 0 or any integer;

R₂ is optionally substituted alkyl, cycloalkyl, aryl, or heteroaryl;

R₄ is optionally substituted

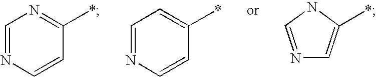

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXV:

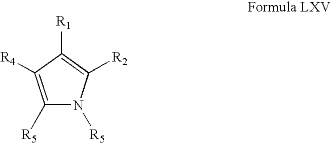

wherein R₁ is

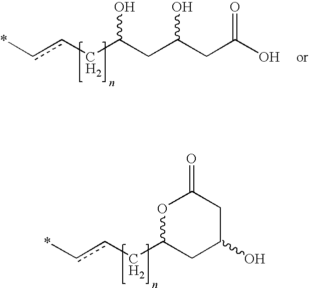

n being 0 or any integer;

R₂ is optionally substituted alkyl, cycloalkyl, aryl, or heteroaryl;

R₄ is optionally substituted

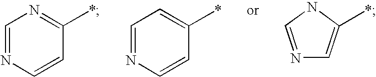

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXV:

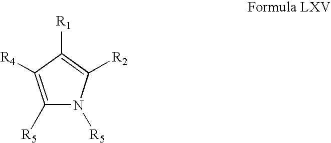

wherein R₁ is

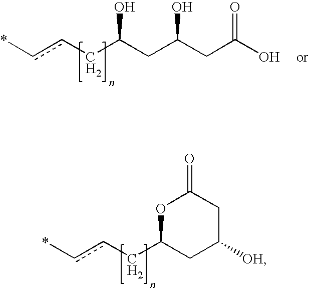

n being 0 or any integer;

R₂ is optionally substituted alkyl, cycloalkyl, aryl, or heteroaryl;

R₄ is optionally substituted

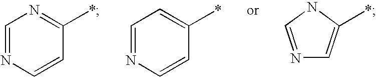

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXVII:

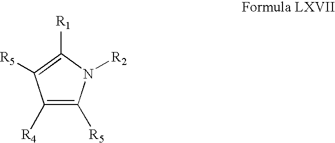

wherein R₁ is

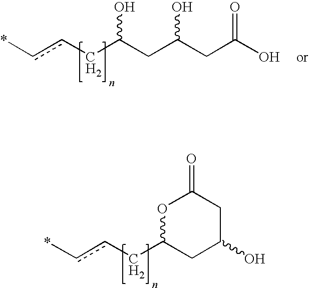

n being 0 or any integer;

R₂ is optionally substituted alkyl, cycloalkyl, aryl, or heteroaryl;

R₄ is optionally substituted

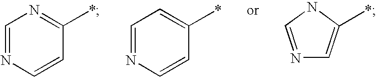

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXVII:

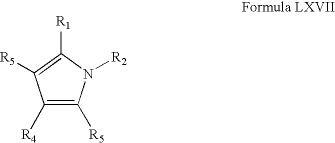

wherein R₁ is

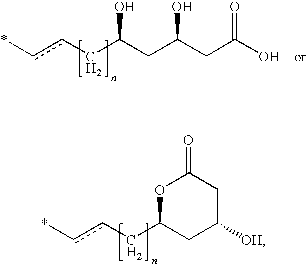

n being 0 or any integer;

R₂ is optionally substituted alkyl, cycloalkyl, aryl, or heteroaryl;

R₄ is optionally substituted

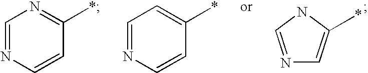

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXVIII:

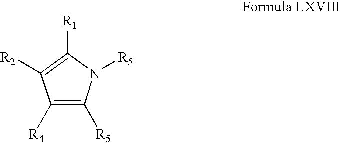

wherein R₁ is

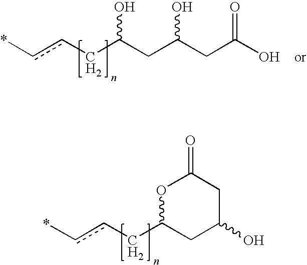

n being 0 or any integer;

R₂ is optionally substituted alkyl, cycloalkyl, aryl, or heteroaryl;

R₄ is optionally substituted

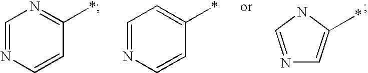

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXVIII:

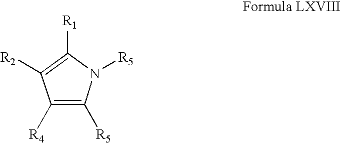

wherein R₁ is

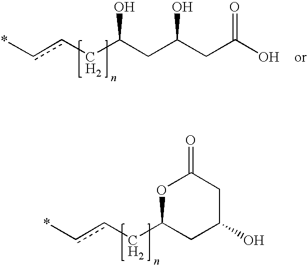

n being 0 or any integer;

R₂ is optionally substituted alkyl, cycloalkyl, aryl, or heteroaryl;

R₄ is optionally substituted

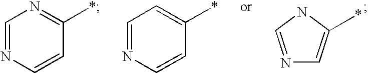

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXIX:

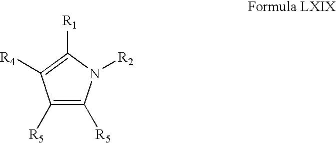

wherein R₁ is

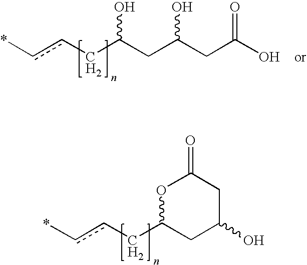

n being 0 or any integer;

R₂ is optionally substituted alkyl, cycloalkyl, aryl, or heteroaryl;

R₄ is optionally substituted

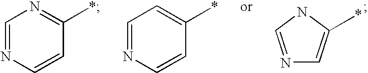

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXIX:

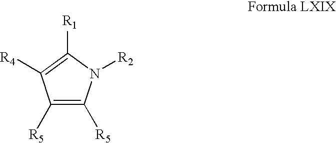

wherein R₁ is

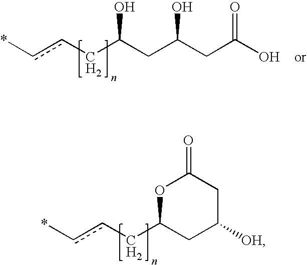

n being 0 or any integer;

R₂ is optionally substituted alkyl, cycloalkyl, aryl, or heteroaryl;

R₄ is optionally substituted

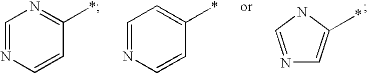

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXI:

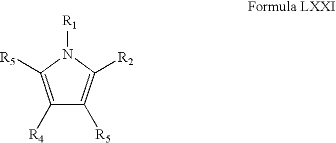

wherein R₁ is

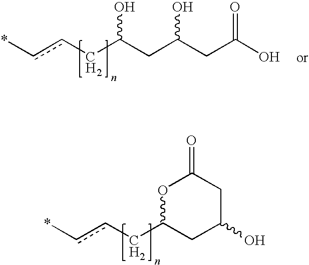

n being 0 or any integer;

R₂ is optionally substituted alkyl, cycloalkyl, aryl, or heteroaryl;

R₄ is optionally substituted

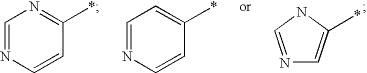

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXI:

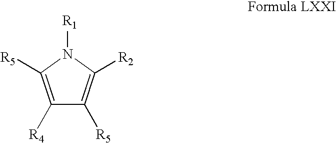

wherein R₁ is

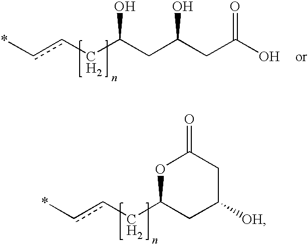

n being 0 or any integer;

R₂ is optionally substituted alkyl, cycloalkyl, aryl, or heteroaryl;

R₄ is optionally substituted

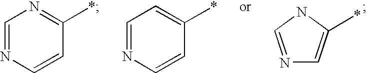

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXII:

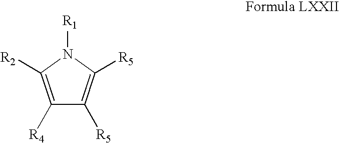

wherein R₁ is

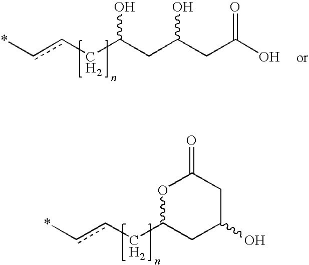

n being 0 or any integer;

R₂ is optionally substituted alkyl, cycloalkyl, aryl, or heteroaryl;

R₄ is optionally substituted

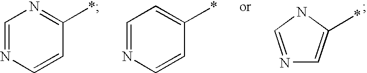

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXII:

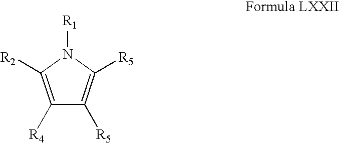

wherein R₁ is

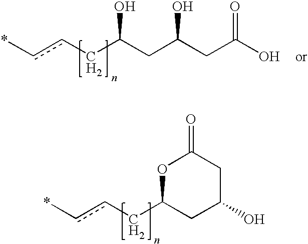

n being 0 or any integer;

R₂ is optionally substituted alkyl, cycloalkyl, aryl, or heteroaryl;

R₄ is optionally substituted

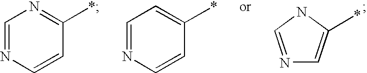

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXIII:

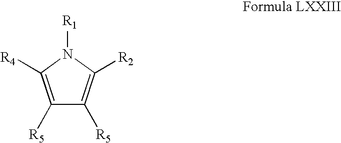

wherein R₁ is

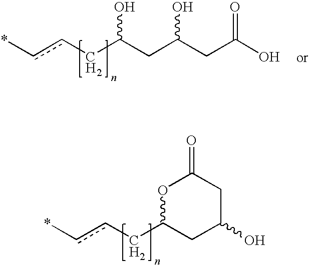

n being 0 or any integer;

R₂ is optionally substituted alkyl, cycloalkyl, aryl, or heteroaryl;

R₄ is optionally substituted

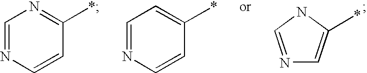

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXIII:

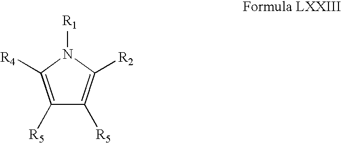

wherein R₁ is

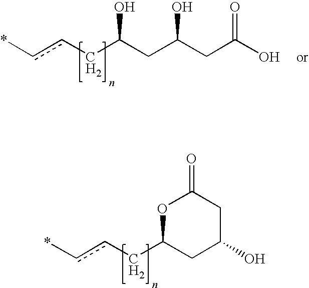

n being 0 or any integer;

R₂ is optionally substituted alkyl, cycloalkyl, aryl, or heteroaryl;

R₄ is optionally substituted

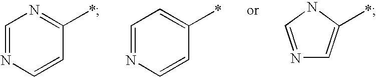

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXX:

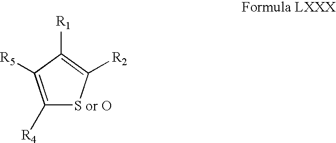

wherein R₁ is

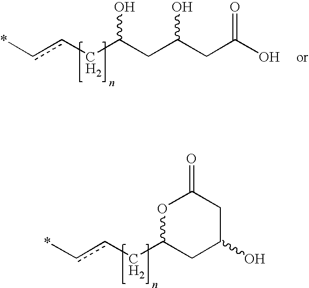

n being 0 or any integer;

R₂ is optionally substituted alkyl, cycloalkyl, aryl, or heteroaryl;

R₄ is optionally substituted

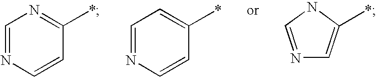

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXX:

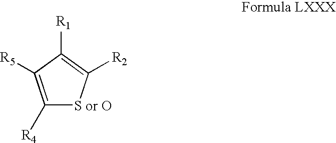

wherein R₁ is

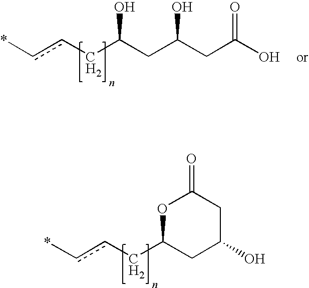

n being 0 or any integer;

R₂ is optionally substituted alkyl, cycloalkyl, aryl, or heteroaryl;

R₄ is optionally substituted

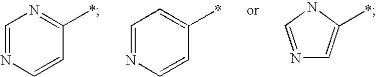

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXI:

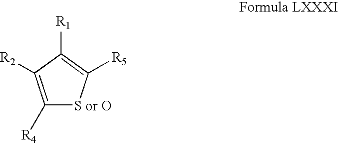

wherein R₁ is

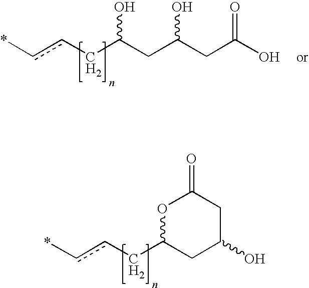

n being 0 or any integer;

R₂ is optionally substituted alkyl, cycloalkyl, aryl, or heteroaryl;

R₄ is optionally substituted

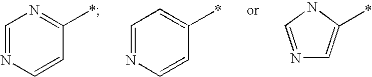

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXI:

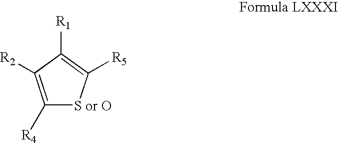

wherein R₁ is

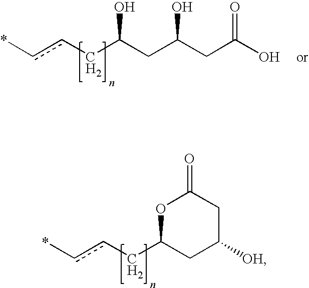

n being 0 or any integer;

R₂ is optionally substituted alkyl, cycloalkyl, aryl, or heteroaryl;

R₄ is optionally substituted

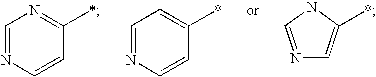

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXII:

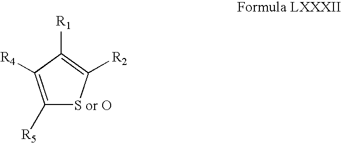

wherein R₁ is

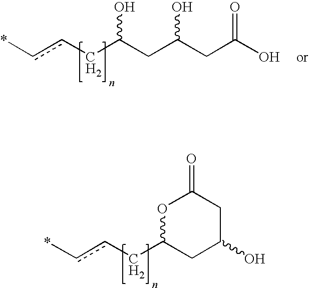

n being 0 or any integer;

R₂ is optionally substituted alkyl, cycloalkyl, aryl, or heteroaryl;

R₄ is optionally substituted

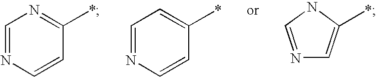

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXII:

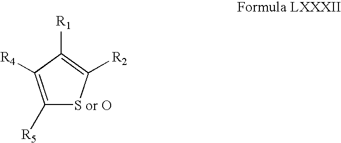

wherein R₁ is

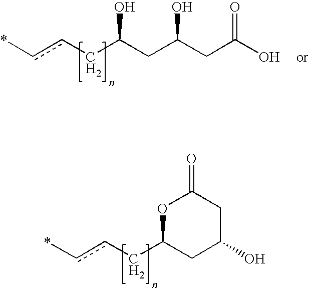

n being 0 or any integer;

R₂ is optionally substituted alkyl, cycloalkyl, aryl, or heteroaryl;

R₄ is optionally substituted

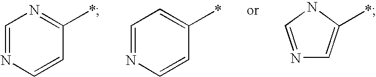

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXIV:

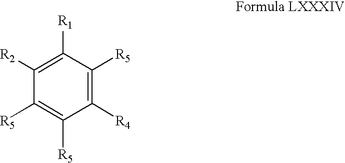

wherein R₁ is

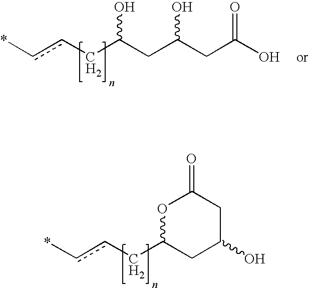

n being 0 or any integer;

R₂ is optionally substituted alkyl, cycloalkyl, aryl, or heteroaryl;

R₄ is optionally substituted

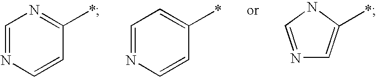

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXIV:

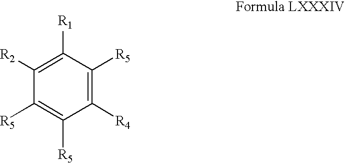

wherein R₁ is

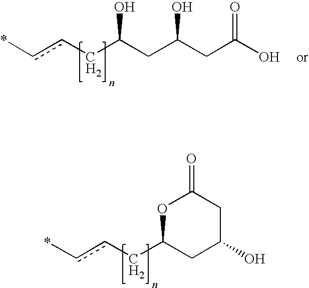

n being 0 or any integer;

R₂ is optionally substituted alkyl, cycloalkyl, aryl, or heteroaryl;

R₄ is optionally substituted

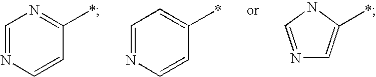

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXV:

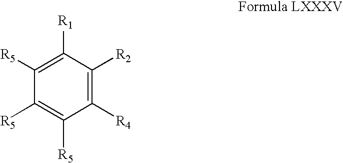

wherein R₁ is

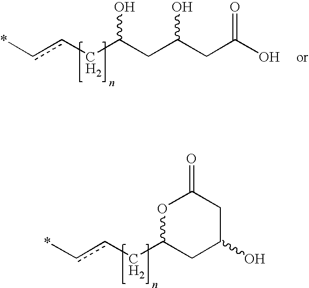

n being 0 or any integer;

R₂ is optionally substituted alkyl, cycloalkyl, aryl, or heteroaryl;

R₄ is optionally substituted

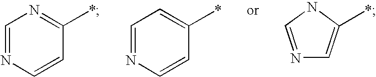

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXV:

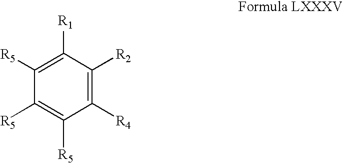

wherein R₁ is

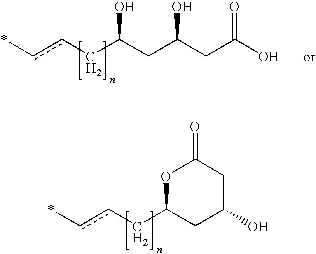

n being 0 or any integer;

R₂ is optionally substituted alkyl, cycloalkyl, aryl, or heteroaryl;

R₄ is optionally substituted

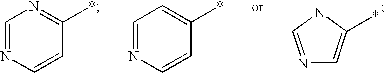

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXVI:

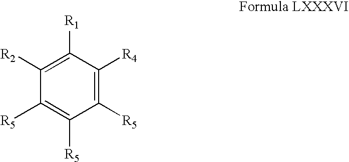

wherein R₁ is

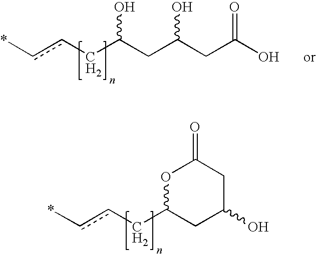

n being 0 or any integer;

R₂ is optionally substituted alkyl, cycloalkyl, aryl, or heteroaryl;

R₄ is optionally substituted

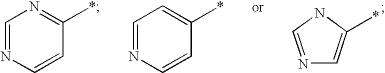

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXVI:

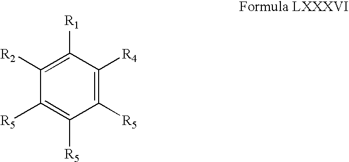

wherein R₁ is

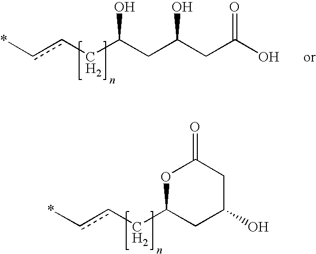

n being 0 or any integer;

R₂ is optionally substituted alkyl, cycloalkyl, aryl, or heteroaryl;

R₄ is optionally substituted

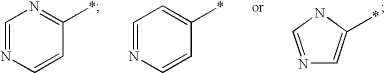

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXVII:

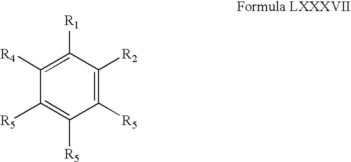

wherein R₁ is

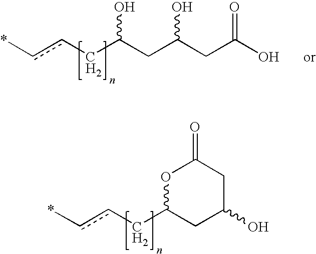

n being 0 or any integer;

R₂ is optionally substituted alkyl, cycloalkyl, aryl, or heteroaryl;

R₄ is optionally substituted

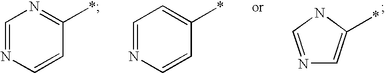

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXVII:

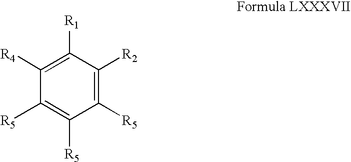

wherein R₁ is

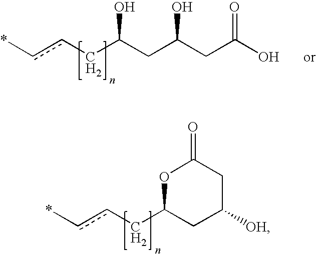

n being 0 or any integer;

R₂ is optionally substituted alkyl, cycloalkyl, aryl, or heteroaryl;

R₄ is optionally substituted

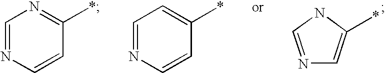

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXVIII:

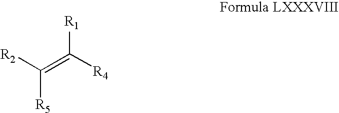

wherein R₁ is

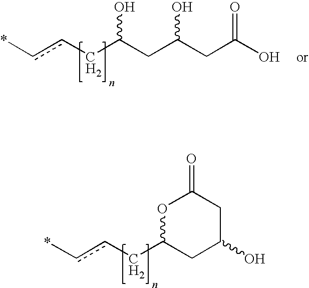

n being 0 or any integer;

R₂ is optionally substituted alkyl, cycloalkyl, aryl, or heteroaryl;

R₄ is optionally substituted

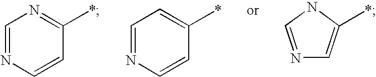

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXVIII:

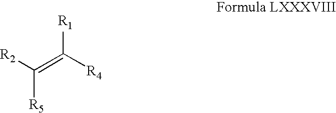

wherein R₁ is

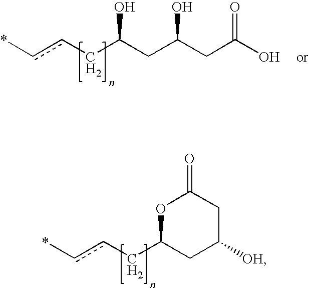

n being 0 or any integer;

R₂ is optionally substituted alkyl, cycloalkyl, aryl, or heteroaryl;

R₄ is optionally substituted

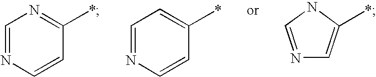

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXIX:

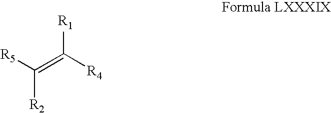

wherein R₁ is

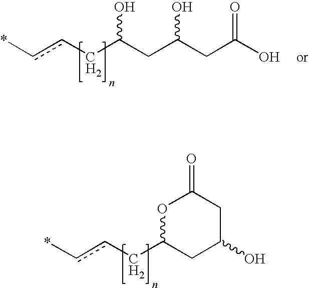

n being 0 or any integer;

R₂ is optionally substituted alkyl, cycloalkyl, aryl, or heteroaryl;

R₄ is optionally substituted

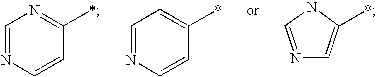

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXIX:

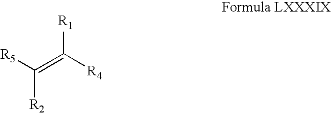

wherein R₁ is

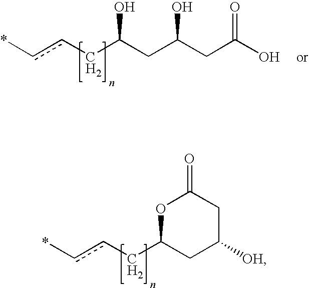

n being 0 or any integer;

R₂ is optionally substituted alkyl, cycloalkyl, aryl, or heteroaryl;

R₄ is optionally substituted

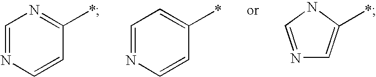

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXX:

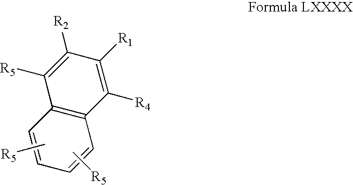

wherein R₁ is

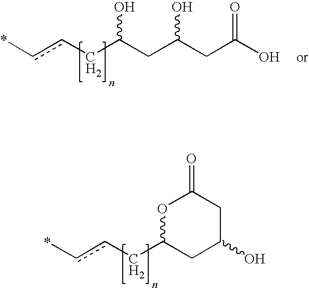

n being 0 or any integer;

R₂ is optionally substituted alkyl, aryl, or heteroaryl;

R₄ is optionally substituted

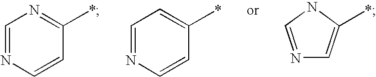

and R₅ is in each instance hydrogen, optionally substituted lower alkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXX:

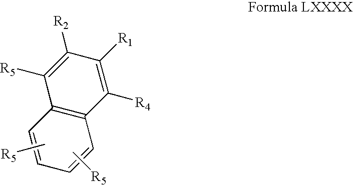

wherein R₁ is

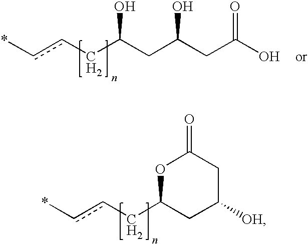

n being 0 or any integer;

R₂ is optionally substituted alkyl, cycloalkyl, aryl, or heteroaryl;

R₄ is optionally substituted

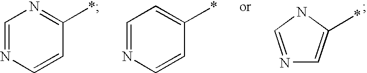

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXI:

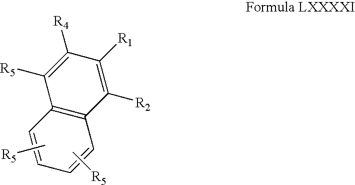

wherein R₁ is

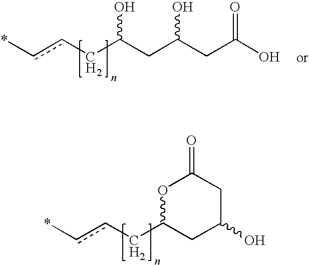

n being 0 or any integer;

R₂ is optionally substituted alkyl, cycloalkyl, aryl, or heteroaryl;

R₄ is optionally substituted

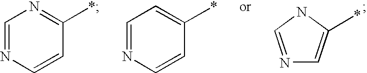

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXXI:

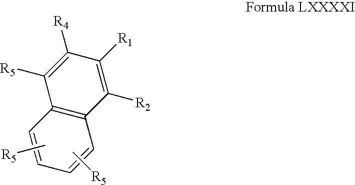

wherein R₁ is

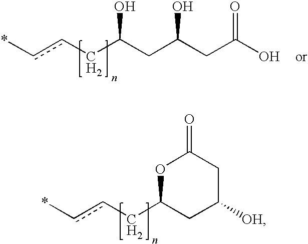

n being 0 or any integer;

R₂ is optionally substituted alkyl, cycloalkyl, aryl, or heteroaryl;

R₄ is optionally substituted

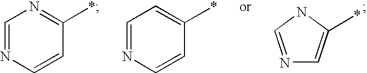

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXII:

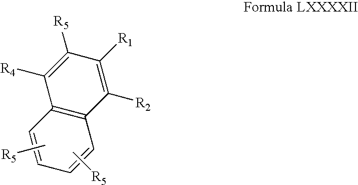

wherein R₁ is

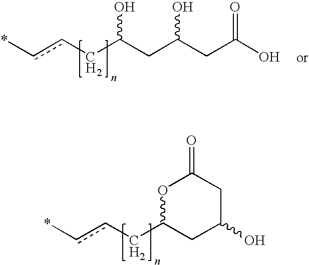

n being 0 or any integer;

R₂ is optionally substituted alkyl, cycloalkyl, aryl, or heteroaryl;

R₄ is optionally substituted

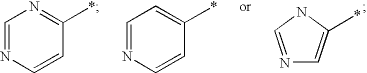

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXXII:

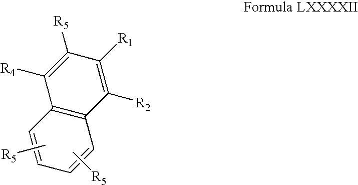

wherein R₁ is

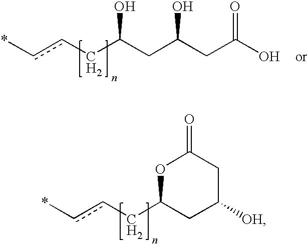

n being 0 or any integer;

R₂ is optionally substituted alkyl, cycloalkyl, aryl, or heteroaryl;

R₄ is optionally substituted

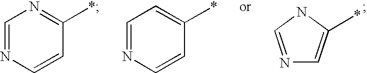

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXIII:

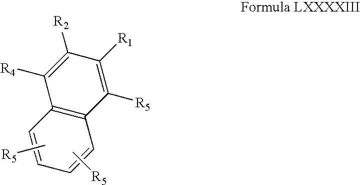

wherein R₁ is

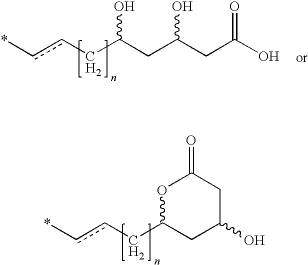

n being 0 or any integer;

R₂ is optionally substituted alkyl, cycloalkyl, aryl, or heteroaryl;

R₄ is optionally substituted

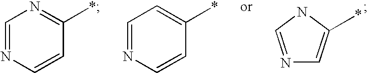

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

Another aspect of the present invention provides a compound comprising formula LXXXXIII:

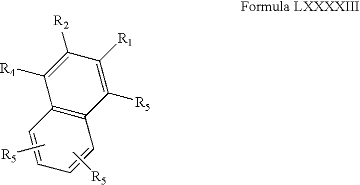

wherein R₁ is

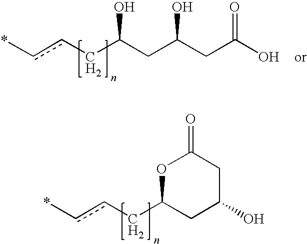

n being 0 or any integer;

R₂ is optionally substituted alkyl, cycloalkyl, aryl, or heteroaryl;

R₄ is optionally substituted

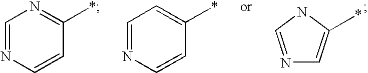

and R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino.

One aspect of the present invention provides methods of inhibiting a MAP kinase comprising administering an effective amount of at least one compound comprising formula LXXXXIV:

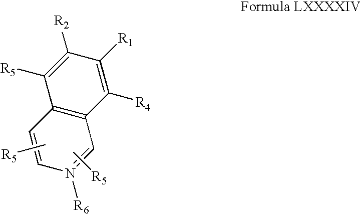

wherein R₁ is

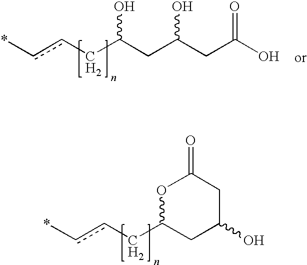

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXIV:

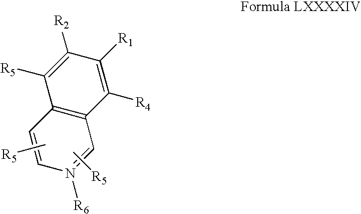

wherein R₁ is

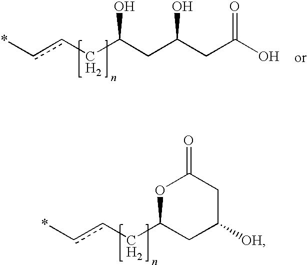

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXV:

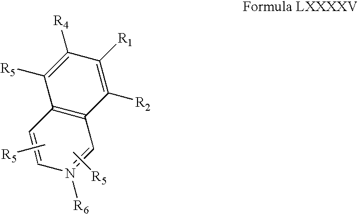

wherein R₁ is

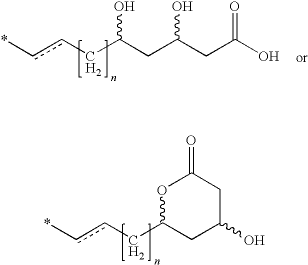

n being u or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXV:

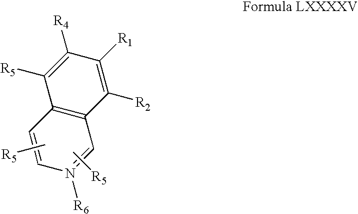

wherein R₁ is

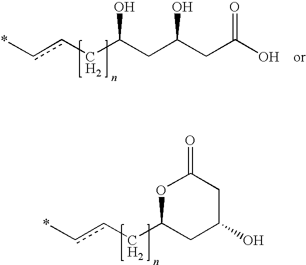

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXVI:

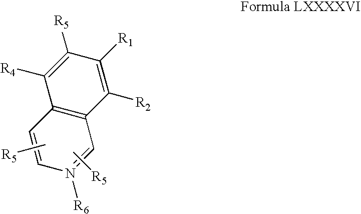

wherein R₁ is

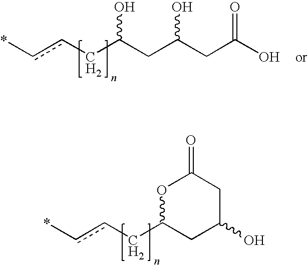

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXVI:

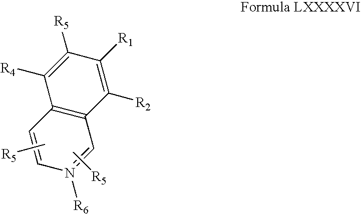

wherein R₁ is

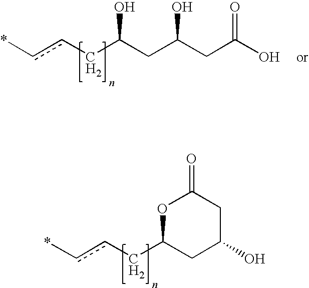

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXVII:

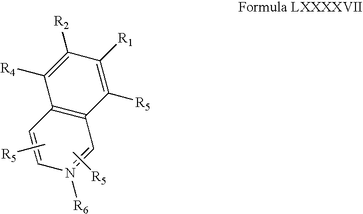

wherein R₁ is

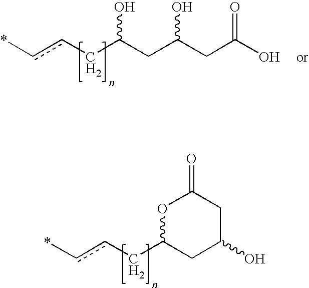

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXVII:

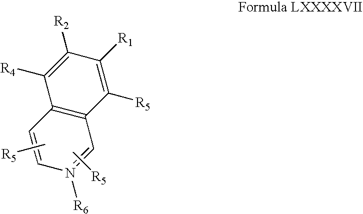

wherein R₁ is

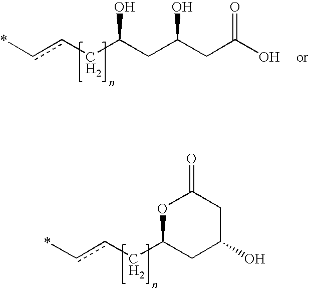

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXVIII:

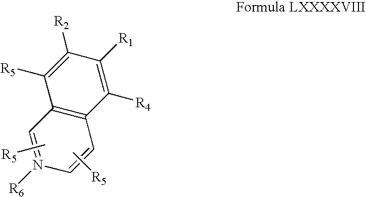

wherein R₁ is

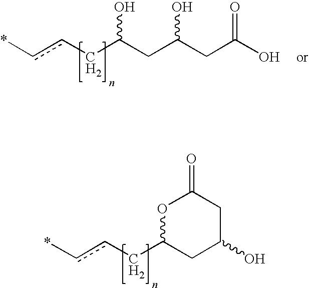

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXVIII:

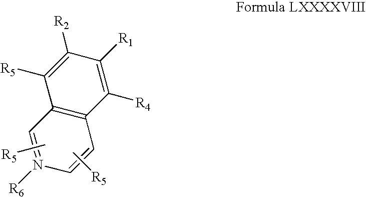

wherein R₁ is

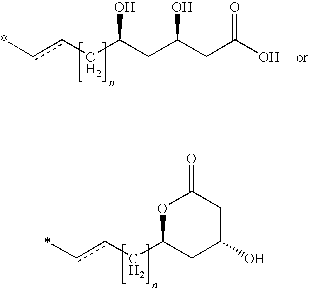

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula LXXXXIX:

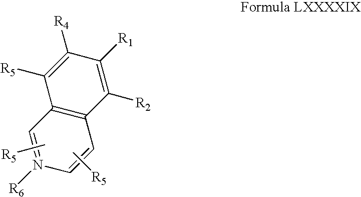

wherein R₁ is

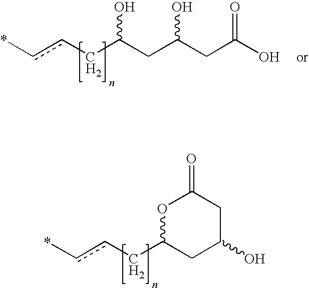

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula LXXXXIX:

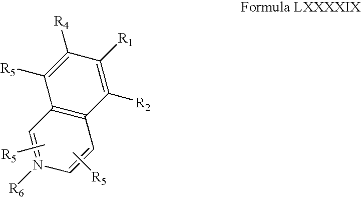

wherein R₁ is

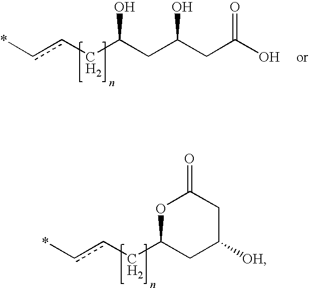

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula C:

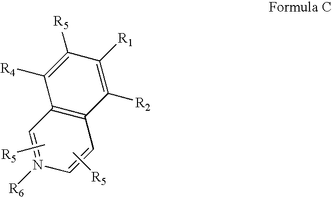

wherein R₁ is

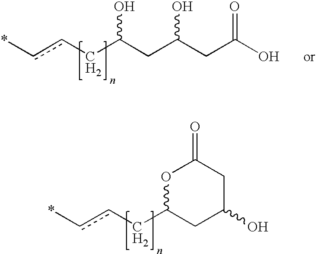

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula C:

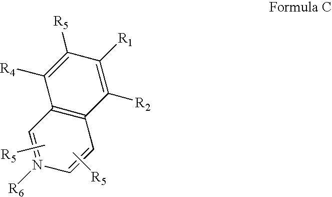

wherein R₁ is

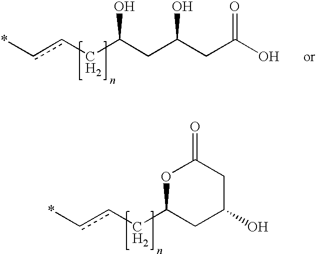

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula CI:

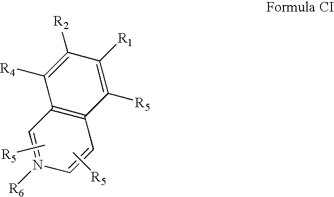

wherein R₁ is

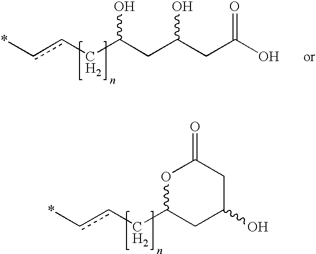

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula CI:

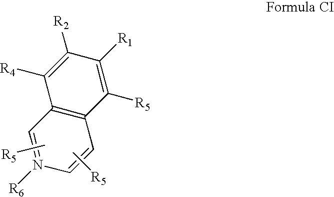

wherein R₁ is

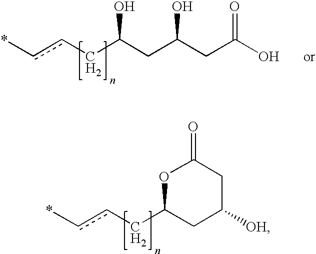

n being 0 or any integer;

R₂ is optionally substituted alkyl, cycloalkyl, aryl, or heteroaryl;

R₄ is optionally substituted alkyl, cycloalkyl, aryl, or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

One aspect of the present invention provides methods of inhibiting a MAP kinase comprising administering an effective amount of at least one compound comprising formula CII:

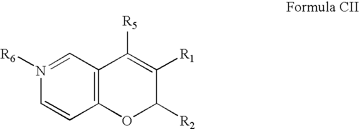

wherein R₁ is

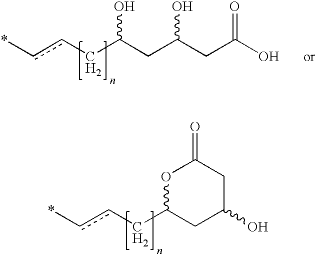

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula CII:

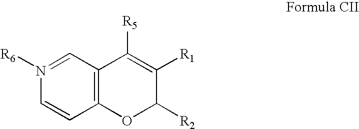

wherein R₁ is

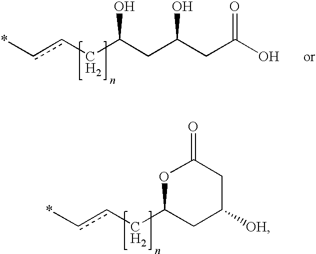

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, optionally substituted amino; and

R₆ is either a lone pair of electrons or an oxygen atom.

One aspect of the present invention provides methods of inhibiting a MAP kinase comprising administering an effective amount of at least one compound comprising formula CIII:

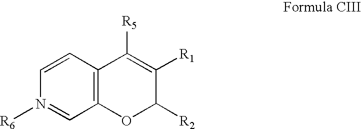

wherein R₁ is

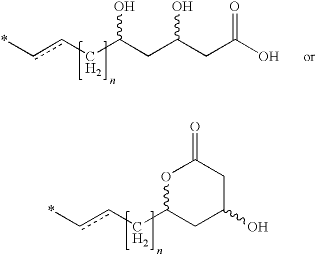

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

Another aspect of the present invention provides a compound comprising formula CIII:

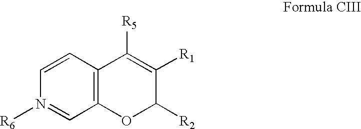

wherein R₁ is

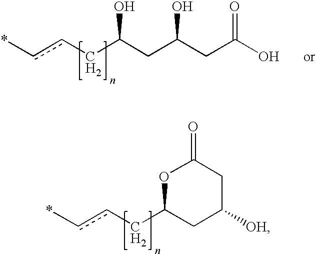

n being 0 or any integer;

R₂ is optionally substituted alkyl, aryl, spirocycle or heteroaryl;

R₅ is in each instance hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino; and

R₆ is either a lone pair of electrons or an oxygen atom.

### III. Methods of Treatment

Another aspect of the present invention relates to methods of using pharmaceutical compositions and kits comprising compounds described herein to treat kinase-related and/or reductase-related compounds, preferably MAP kinase-related and/or HMG-CoA reductase-related conditions, as well as novel uses of known compounds for the treatment of MAP kinase-related conditions, and novel combinations for the treatment of MAP kinase- and/or HMG CoA reductase-related conditions, especially inflammatory conditions.

The present invention provides methods, pharmaceutical compositions, and kits for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying disorder being treated. For example, in an arthritic patient, therapeutic benefit includes eradication or amelioration of the underlying arthritis. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, a MAP kinase inhibitor of the present invention provides therapeutic benefit not only when rheumatoid arthritis is eradicated, but also when an improvement is observed in the patient with respect to other disorders or discomforts that accompany rheumatoid arthritis, like stiffness or swelling in the joints. Similarly, compositions of the present invention can provide therapeutic benefit in ameliorating other symptoms associated with MAP kinase-related conditions, e.g., inflammatory and/or autoimmune conditions, including redness, rashes, swelling, itching, irritation, dryness, scaling, flaking, pain, temperature increase, loss of normal function, and the like.

For prophylactic benefit, a pharmaceutical composition of the invention may be administered to a patient at risk of developing a MAP kinase-related condition and/or a HMG-CoA reductase-related condition, or to a patient reporting one or more of the physiological symptoms of such conditions, even though a diagnosis of the condition may not have been made. Administration may prevent the condition from developing, or it may reduce, lessen, shorten and/or otherwise ameliorate the condition that develops.

### A. Treatment of MAP Kinase-Related Conditions

The term “MAP kinase-related condition” as used herein refers to a condition in which directly or indirectly reducing the activity of a protein kinase involved in signaling cascades of an allergic, inflammatory and/or an autoimmune response is desirable, and/or directly or indirectly reducing the production and/or effects of one or more products of the protein kinase is desirable. For example, a MAP kinase-related condition may involve over-production or unwanted production of one or more pro-inflammatory cytokines, such as tumor necrosis factor-α (TNF-α), interleukin-1β (IL-1β), or other chemical messengers of signal transduction pathways associated with inflammation (including responses to and expression of TNF-α and IL-1β), apoptosis, growth and differentiation.

Examples of MAP kinase-related conditions include but are not limited to allergic, inflammatory and autoimmune conditions, such as, for example, ocular allergic, ocular inflammatory and/or ocular autoimmune conditions; allergic, inflammatory and/or autoimmune conditions of the ear; allergic, inflammatory and/or autoimmune conditions of the skin and skin structures; gastrointestinal allergic, gastrointestinal inflammatory and/or gastrointestinal autoimmune conditions; respiratory allergic, respiratory inflammatory and/or respiratory autoimmune conditions; as well as arthritis, rheumatoid arthritis, and/or other inflammatory/autoimmune diseases of the musculoskeletal system; osteoarthritis; vascular inflammatory conditions, vasculitis, inflammatory bowel disease (including ulcerative colitis and Crohn's disease), Celiac sprue, acne, psoriasis (as well as other papulosquamous disorders such as lichen planus), topical dermatitis; atopic dermatitis (including eczema), irritant contact dermatitis, endotoxemia, restenosis, sepsis, and toxic shock syndrome, as well as transplant rejection. Other MAP kinase-related conditions include ageing, photo-ageing, cachexia, leprosy, Leishmaniasis, asthma, chronic pelvic pain, inflammatory muscle disease, allergic rhinitis (hay fever), gastritis, vaginitis, conjunctivitis, interstitial cystitis, chronic fatigue syndrome, osteoporosis, scleroderma, and the like. MAP-kinase related conditions can also include diabetes, chronic obstructive pulmonary disease, as well as cardiovascular-related conditions such as atherosclerosis, myocardial infarction, congestive heart failure, ischemic-reperfusion injury and other vascular inflammatory conditions. MAP-kinase related conditions can also include proliferative disorders, including cancers, e.g., multiple myeloma, fibrotic disorders, mesangial cell proliferative disorders, such as glomerulonephritis, diabetic nephropathy malignant nephrosclerosis, thrombotic microangiopathy syndromes, organ transplant rejection and glomerulopathies. MAP-kinase related condition can also include neurodegenerative diseases, e.g. Alzheimer's and pain sensation, as well as infectious diseases such as viral, bacterial, and fungal infections.

Other conditions treatable with compositions, kits, and methods of the present invention include those currently treated with soluble TNF receptors, anti-TNF antibodies, IL-1 receptor antagonists, TNF-α converting enzyme inhibitors, inhibitors of protein-tyrosine kinases and/or inhibitors of protein serine/threonine kinases of the MAP kinase family, preferably including conditions currently treated with inhibitors of p38 MAP kinases and/or the stress-activated protein kinases/Jun N-terminal kinases (SAPKs/JNKs). Most preferably, conditions treatable with the practice of this invention include those relating to p38αMAP kinase, e.g, conditions currently treated by inhibition of p38αMAP kinase activity.

Reducing the activity of a protein kinase, e.g. a MAP kinase, is also referred to as “inhibiting” the kinase. The term “inhibits” and its grammatical conjugations, such as “inhibitory,” do not require complete inhibition, but refer to a reduction in kinase activity. Such reduction is preferably by at least about 50%, at least about 75%, at least about 90%, and more preferably by at least about 95% of the activity of the enzyme in the absence of the inhibitory effect, e.g., in the absence of an inhibitor. Conversely, the phrase “does not inhibit” and its grammatical conjugations refer to situations where there is less than about 20%, less than about 10%, and preferably less than about 5%, of reduction in enzyme activity in the presence of the compound. Further the phrase “does not substantially inhibit” and its grammatical conjugations refer to situations where there is less than about 30%, less than about 20%, and preferably less than about 10% of reduction in enzyme activity in the presence of the compound.

The ability to reduce enzyme activity is a measure of the potency or the activity of a compound, or combination of compounds, towards or against the enzyme. Potency is preferably measured by cell free, whole cell and/or in vivo assays in terms of IC50, Kᵢ and/or ED50 values. An IC50 value represents the concentration of a compound required to inhibit enzyme activity by half (50%) under a given set of conditions. A Kᵢ value represents the equilibrium affinity constant for the binding of an inhibiting compound to the enzyme. An ED50 value represents the dose of a compound required to effect a half-maximal response in a biological assay. Further details of these measures will be appreciated by those of ordinary skill in the art, and can be found in standard texts on biochemistry, enzymology, and the like.

In some embodiments, compounds in one or more forms represented herein inhibit a MAP kinase. These compounds can exert anti-inflammatory effects in vitro and/or in vivo and can form the basis for pharmaceutical compositions useful in the treatment of MAP kinase-related conditions, e.g., allergic, inflammatory and/or autoimmune diseases, in humans and other mammals. In certain embodiments, for example, these compositions reduce production of, and signaling pathways involving, TNF-α and IL-1β.

The present invention also includes kits that can be used to treat a MAP kinase-related conditions. These kits comprise a compound or combination of compounds described herein and preferably instructions teaching the use of the kit according to the various methods and approaches described herein. Such kits also include information, such as scientific literature references, package insert materials, clinical trial results, and/or summaries of these and the like, which indicate or establish the activities and/or advantages of the compound. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

### B. Treatment of HMG-CoA Reductase-Related Conditions

The term “HMG-CoA reductase-related condition” as used herein refers to a condition in which directly or indirectly reducing the activity of HMG-CoA reductase is desirable and/or directly or indirectly reducing the production and/or effects of one or more products of HMG-CoA reductase is desirable. For example, an HMG-CoA reductase-related condition may involve elevated levels of cholesterol, in particular, non-HDL cholesterol in plasma, such as elevated levels of LDL cholesterol. Typically, a patient is considered to have high or elevated cholesterol levels based on a number of criteria, for example, see Pearlman, Postgrad. Med. 112(2): 13-26 (2002), incorporated herein by reference. Guidelines include serum lipid profiles, such as LDL compared with HDL levels.

Examples of HMG-CoA reductase-related conditions include hypercholesterolemia, lipid disorders such as hyperlipidemia, and atherogenesis and its sequelae of cardiovascular diseases, including atherosclerosis, other vascular inflammatory conditions, myocardial infarction, ischemic stroke, occlusive stroke, and peripheral vascular diseases, as well as other conditions in which decreasing cholesterol and/or other products of the cholesterol biosynthetic pathways can produce a benefit. Other HMG-CoA reductase-related conditions treatable with compositions, kits, and methods of the present invention include those currently treated with statins.

Reducing the activity of HMG-CoA reductase, is also referred to as “inhibiting” the enzyme. The term “inhibits” and its grammatical conjugations, such as “inhibitory,” do not require complete inhibition, but refer to a reduction in HMG-CoA reductase activity. Such reduction is preferably by at least about 50%, at least about 75%, at least about 90%, and more preferably by at least about 95% of the activity of the enzyme in the absence of the inhibitory effect, e.g., in the absence of an inhibitor. Conversely, the phrase “does not inhibit” and its grammatical conjugations refer to situations where there is less than about 20%, less than about 10%, and preferably less than about 5% of reduction in enzyme activity in the presence of the compound. Further the phrase “does not substantially inhibit” and its grammatical conjugations refer to situations where there is less than about 30%, less than about 20%, and preferably less than about 10% of reduction in enzyme activity in the presence of the compound.

The ability to reduce enzyme activity is a measure of the potency or the activity of the compound or combination of compounds towards or against the enzyme. Potency is preferably measured by cell free, whole cell and/or in vivo assays in terms of IC50 or ED50 values. An IC50 value represents the concentration of a compound required to inhibit the enzyme activity by half (50%) under a given set of conditions. A Ki value represents the equilibrium affinity constant for the binding of an inhibiting compound to the enzyme. An ED50 value represents the dose of a compound required to effect a half-maximal response in a biological assay. Further details of these measures will be appreciated by those of ordinary skill in the art, and can be found in standard texts on biochemistry, enzymology, and the like.

In some embodiments, compounds represented herein inhibit HMG-CoA reductase. Such compounds find use in the practice of this invention e.g., in a method of treating an HMG-CoA reductase-related condition by administering to a subject an effective amount of at least one of such compounds. These compounds can lower cholesterol levels in vitro and in vivo, and/or increase HDL, thereby forming the basis for pharmaceutical compositions useful in the treatment of HMG-CoA reductase-related conditions, e.g., hypercholesterolemia and atherosclerosis, in humans and other mammals.

Also as noted above, in some embodiments, a compound of the instant invention, or a composition comprising one or more such compounds, can be used in treating an HMG-CoA reductase-related condition by increasing HDL levels. Higher levels of HDL are believed to protect against, e.g., atherosclerosis, whereas low HDL is recognized as an independent risk factor for coronary artery disease. For example, an HDL level below about 40 mg/DL can be considered in need of treatment. Without being limited to a particular theory and/or hypothesis, compounds of the instant invention can bring about an upregulation of HDL in treating an HMG Co-A reductase related condition, such as artherosclerosis.

Upregulating HDL is also referred to as “increasing” HDL or HDL levels. The term “increases” and its grammatical conjugations can refer to a small, significant and/or substantial increase, preferably an increase sufficient to decrease a risk of an HMG-CoA reductase-related condition in a subject being treated. Such increase is preferably by at least about 10%, at least about 20%, at least about 30%, and more preferably by at least about 50% of HDL levels in the absence of treatment. In preferred embodiments, atorvastatin and analogs of atorvastatin are used to increase HDL.

The present invention also includes kits that can be used to treat an HMG-CoA reductase-related condition. These kits comprise a compound or combination of compounds described herein, and preferably instructions teaching the use of the kit according to the various methods and approaches described herein. Such kits also include information, such as scientific literature references, package insert materials, clinical trial results, and/or summaries of these and the like, which indicate or establish the activities and/or advantages of the compound(s). Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

### C. Treatment of Both MAP Kinase- and HMG-CoA Reductase-Related Conditions

One of the purposes of this invention is to describe compounds or combinations of compounds which inhibit both MAP kinase and HMG-CoA reductase. Such compounds or combinations can exert concomitant anti-inflammatory and cholesterol-lowering effects in vitro and/or in vivo. In certain embodiments, for example, these compounds or combinations reduce production of, and signaling pathways involving, TNF-α and IL-1β, as well as inhibiting production of cholesterol and/or other downstream products of mevalonate, including mevalonate pyrophosphate, isopentyl pyrophosphate, geranyl pyrophosphate, farnesyl pyrophosphate, dolichols, farnesylated proteins, trans-trans geranylgeranyl pyrophosphate, ubiquinone, geranyl-geranylated proteins, squalene, and the like. Further, in some embodiments, these compounds or combinations can exert superior anti-atherogenesis and/or anti-inflammatory effects in vivo.

Such compounds or combinations can form the basis for pharmaceutical compositions, kits, and methods for treating both MAP kinase-related conditions and HMG-CoA reductase-related conditions in humans and other animals. Moreover, such compositions can provide superior benefits in treating HMG-CoA reductase-related conditions, such as cardiovascular disease, compared with treatments that inhibit HMG-CoA reductase but do not inhibit or do not substantially inhibit MAP kinase. Also, compositions of the present invention can provide superior benefits in treating MAP kinase-related conditions, such as inflammatory conditions, compared with treatments that inhibit MAP kinases but do not inhibit or do not substantially inhibit HMG-CoA reductase.

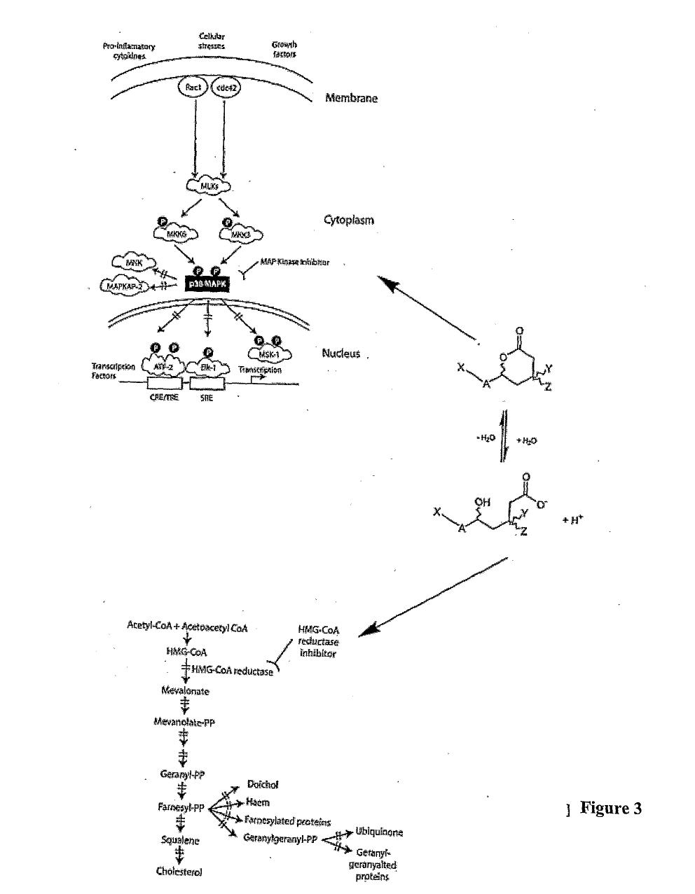

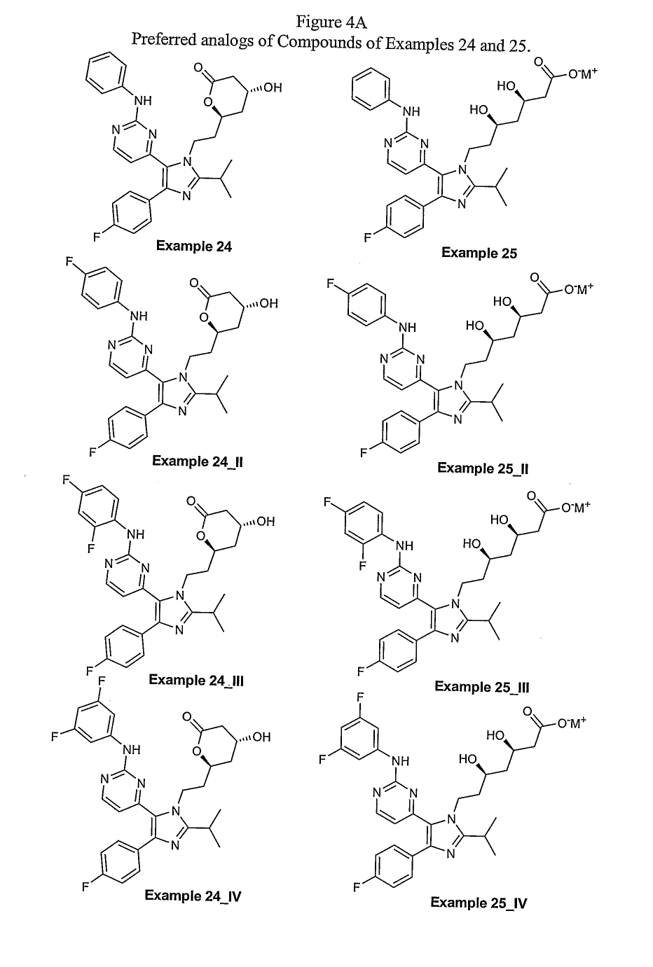

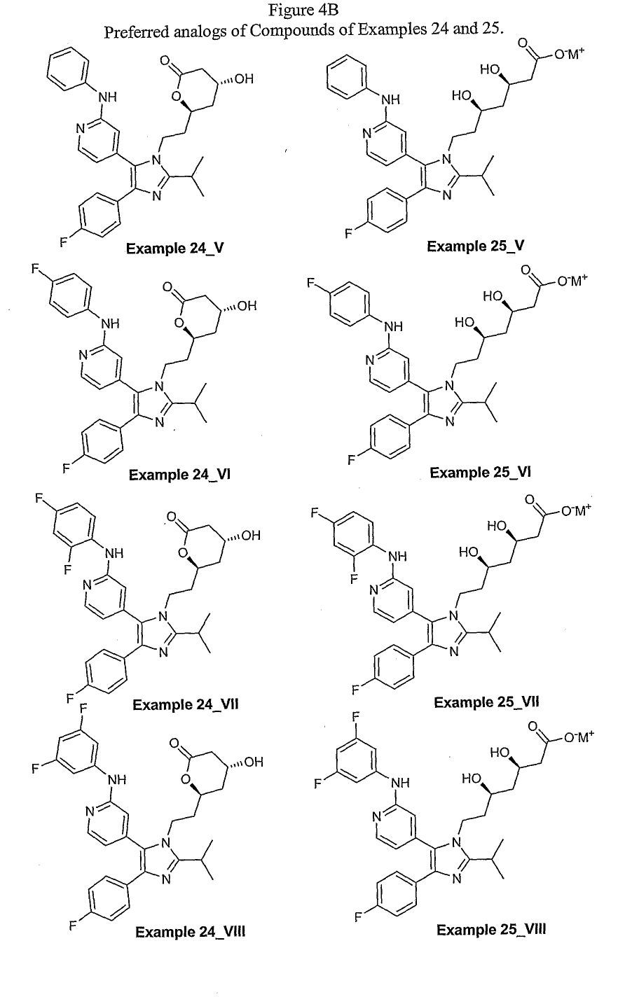

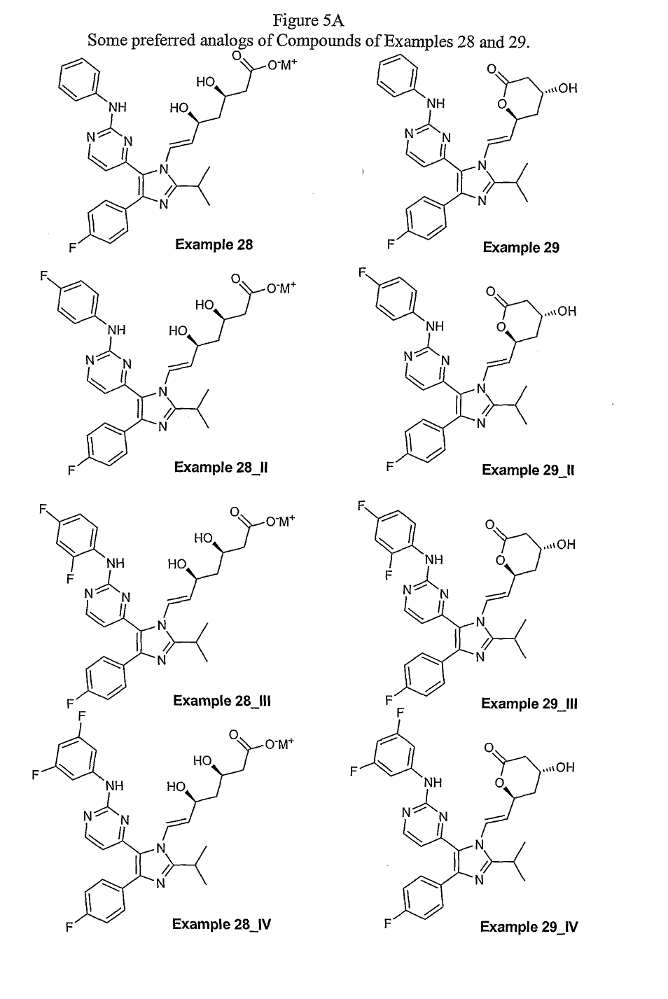

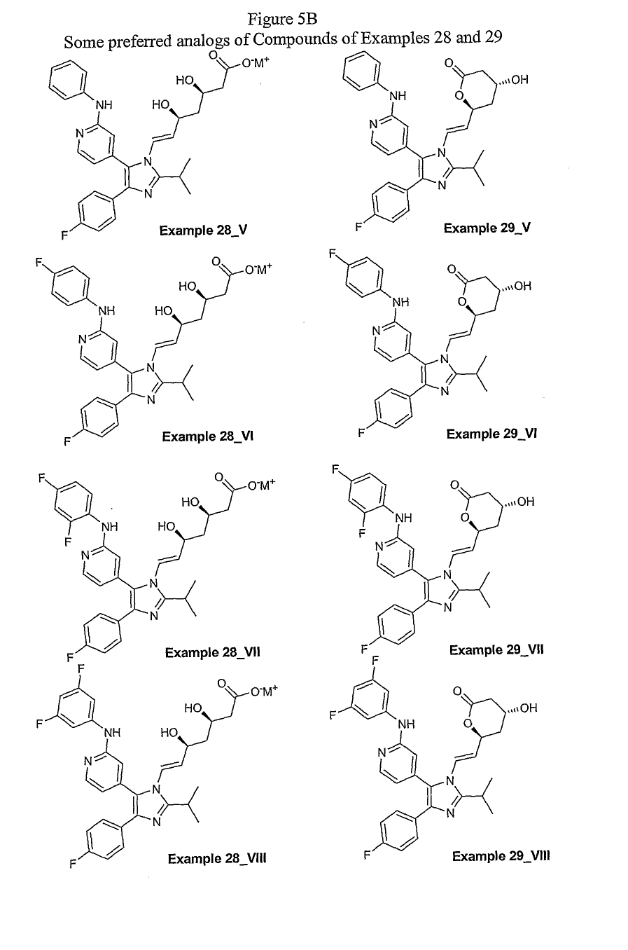

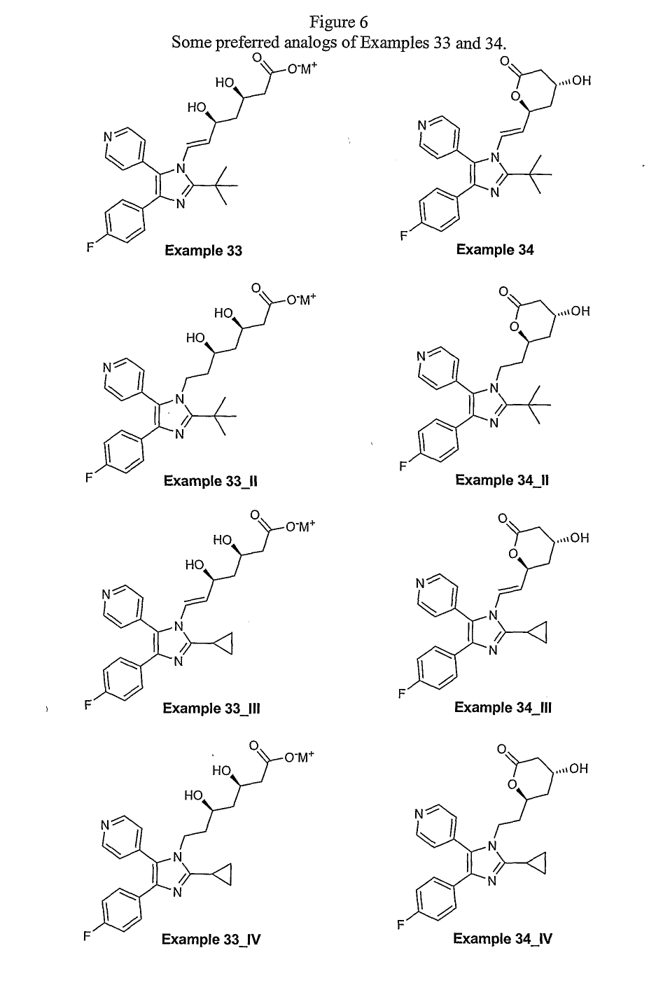

FIG. 3, for example, illustrates a treatment approach in which compositions of the present invention produce a benefit in both MAP kinase- and HMG-CoA reductase-related conditions. This figure serves only as an example, and is in no way intended to be limiting with respect to the present invention. For example, those skilled in the art will readily appreciate variations and modifications of the scheme illustrated, and such variations and modifications are also contemplated as being contained within the scope of the invention.

As FIG. 3 illustrates, the δ-lactone form of a compound of this invention can inhibit a MAP kinase, and the acid form, in particular the deprotonated carboxylate form, can inhibit HMG-CoA reductase. Accordingly, this treatment approach can provide a benefit in both a HMG-CoA reductase-related condition and a MAP kinase-related condition, for instance, in a method comprising administering to a subject an effective amount of at least one of such compounds, e.g., reducing pro-inflammatory cytokine production, in the treatment of a MAP kinase-related condition, such as an allergic, inflammatory and/or autoimmune condition, and reducing cholesterol production in the treatment of a HMG-CoA reductase-related condition, such as cardiovascular disease. This reduction in pro-inflammatory cytokine production by inhibition of a MAP kinase, e.g., p38αMAP kinase, may be in addition to other immunomodulatory effects of some HMG-CoA reductase inhibitors that may, for example, produce immunomodulatory responses though the action of metabolites such as farnesyl pyrophosphate and/or geranylgeranyl pyrophosphate. Moreover, in some embodiments, the role of a compound of the present invention in a MAP kinase-related pathway is distinct from the anti-inflammatory effects of some statins through metabolite products such as geranylgeranyl pyrophosphate and/or farnesyl pyrophosphate. For example, the inhibitory activity of some compounds of this invention on a MAP kinase and on MAP-kinase related conditions need not be reversed by exogenous addition of mevalonate (e.g., sodium mevalonate), geranylgeranyl pyrophosphate, farnesyl pyrophosphate, and/or other downstream product of mevalonate.

Furthermore, the interplay between inflammatory and HMG-CoA reductase-related disorders means that compositions regulating both a MAP kinase and HMG-CoA reductase pathways can be particularly beneficial. Inhibition of HMG-CoA reductase can lead to improved serum lipid profiles, such as decreased LDL and increased HDL levels, which in turn can lead to a reduction in the rate of atherogenesis. On the other hand, initiation of atherogenic plaque deposition (e.g., via foam cells) is reduced by the anti-inflammatory effects, including those which derive from inhibition of a MAP kinase. Inhibition of a MAP kinase can also antagonize inflammatory processes which contribute to the disruption of atherogenic plaques and which, in turn, can lead to arterial thrombosis, blockade, etc.

The present invention also includes kits that can be used to treat MAP kinase- and HMG-CoA reductase-related conditions, in particular cardiovascular disease related to atherogenesis. These kits can comprise a compound or combination of compounds described herein, which have inhibitory activity against both a MAP kinase and HMG-CoA reductase, and preferably instructions teaching the use of the kit according to the various methods and approaches described herein.

Such kits also include information, such as scientific literature references, package insert materials, clinical trial results, and/or summaries of these, and the like, which indicate or establish the multiple activities of the compounds or combination and indicate and/or establish how its use provides advantages and/or differential superiority in treating an HMG-CoA reductase- and/or a MAP kinase-related condition, preferably in treating cardiovascular disease. Such information may be based on the results of various studies, for example, studies using experimental animals involving in vivo models and studies based on human clinical trials. Kits of the present invention may also include materials comparing the approaches of the present invention with other therapies, which do not display a combination of MAP kinase plus HMG-CoA reductase inhibitory activities. Kits described herein can be provided, marketed and/or promoted to health providers, including physicians, nurses, pharmacists, formulary officials, and the like.

### D. Treatment of Inflammatory Conditions

Another aspect of the present invention relates to methods of using pharmaceutical compositions and kits comprising combinations of compounds, forms and/or agents described herein to treat MAP kinase-related and/or HMG-CoA reductase-related conditions that are inflammatory conditions. Inflammatory conditions, as used herein, can refer to inflammatory diseases or disorders associated with inflammation due to relatedness to MAP kinase-, HMG CoA reductase- and/or other pathways. Inflammatory conditions treatable using some embodiments of the instant invention can involve different organ systems, and can vary in severity from trivial to lethal.

For example, inflammatory conditions of the skin can be treated in some embodiments of the instant invention, including, but not limited to, atopic dermatitis, age-related effects, acne, eczema, psoriasis and skin cancer. Various types of acne can be treated using some embodiments of the instant invention, including, e.g., acne atrophica, bromide or chlorine acne, common acne (acne vulgaris), acne conglobata, contact acne, contagious acne of horses, acne cosmetica, cystic acne, acne detergicans, epidemic acne, acne estivalis, excoriated acne, acne frontalis, acne fulminans, halogen acne, acne indurata, infantile acne, iodide acne, acne keloid, acne mechanica, acne necrotica miliaris, neonatal acne, acne papulosa, picker's acne, pommade acne, premenstrual acne, acne pustulosa, acne rosacea, acne scorbutica, acne scrofulosorum, acne tropicalis, acne urticata, acne varioliformis, acne venenata, and the like.

Various types of eczema can be treated in some embodiments of the instant invention, including, e.g., eczematous dermatitis, such as atopic dermatitis, the most common form of eczema, generally seen in infants and young adults. Eczema can present as a red, itchy, non-contagious inflammation of the skin that can be acute or chronic, possibly accompanied by red skin patches, pimples, crusts, scabs, and watery discharge.

Various effects of ageing can be treated in some embodiments of the instant invention, including, e.g., skin-related inflammatory diseases attributable to ageing. Such effects can include formation of wrinkles and fine lines, slackening of cutaneous and subcutaneous tissue, loss of skin elasticity, reduction in skin tone and texture and/or yellowing. Loss of elasticity can result form atrophy of the epidermis, beginning on a small scale and eventually decreasing the number of cells in the dermis. Capillaries can become more susceptible to bruising, collagen metabolism may slow, and/or the concentration of the cell surface molecule glycosaminoglycan (believed to have a role in the recognition of other cells and substrates) may decrease. With ageing, skin may exhibit chronic inflammation with enlarged fibroblasts. Effects of ageing aggravated with sun exposure can also be treated in some embodiments, e.g., pigmentation marks, telangiectasias, elastosis, and/or other skin photo-damage, as well as benign, premalignant and/or malignant neoplasms (e.g., caused by prolonged sun exposure). Ageing itself, e.g., can be considered as an inflammatory condition, e.g., as the ability to mount an inflammatory response decreases and healing time for injuries increases with age.

Inflammatory conditions of the skin that can be treated in some embodiments of the instant invention include skin cancers and other hyperproliferative skin disorders, including, e.g., without being not limited to, basal cell carcinoma, squamous cell carcinoma (Bowen's disease), keratosis (such as actinic or seborrheic keratosis), and/or disorders of keratinization (such as ichthyosis and keratoderma).

Inflammatory conditions of the respiratory system can be treated in some embodiments of the instant invention, including, but not limited to, allergic rhinitis, chronic obstructive pulmonary disease, adult respiratory distress syndrome, asthma, and the like. Allergic asthma can include atopic, chronic diseases of the lung characterized by inflammation of the air passages. Allergic rhinitis or hay fever can include conditions that affect mucous membranes characterized by seasonal or perennial nasal inflammation, e.g., in response to an allergen. Other mucous inflammatory conditions treatable using some embodiments of the instant invention can include lamellar ichthyosis, acne, rosacea, and the like.

Inflammatory conditions of the urogenital tract can be treated in some embodiments of the instant invention, including, but not limited to, vaginitis and interstitial cystitis, and other conditions characterized by inflammation of the urogenital epithelium and/or the urinary bladder. Interstitial cystitis also can include other conditions associated with a dysfunctional bladder glycosaminoglycan protective layer and/or increased numbers of activated bladder mast cells.

Inflammatory conditions of the gastrointestinal tract can be treated in some embodiments of the instant invention, including, but not limited to, celiac diseases, e.g., celiac sprue, inflammatory bowel disease (including ulcerative colitis and Crohn's disease), intestinal infections, enterocolitis, gastritis, and the like.

Inflammatory conditions of the musculoskeletal system can be treated in some embodiments of the instant invention, including, but not limited to, inflammatory muscle pain, arthritis, rheumatoid arthritis, psoriatic arthritis, osteoarthritis, osteoporosis, and the like. Osteoporosis can include conditions characterized by decreased bone mass, increased fragility of the remaining bone, and/or increased incidence of fractures.

Inflammatory conditions of the vascular system an be treated in some embodiments of the instant invention, including, but not limited to hypercholesterolemia, hyperlipidemia, atherogenesis and associated cardiovascular risks of atherosclerosis, thrombosis, myocardial infarction, ischemic stroke, ischemic-reperfusion injury, peripheral vascular disease, e.g., peripheral occlusive disease, and the like. Inflammatory conditions of the systemic circulation also include endotoxemia, lupus erythrematosus, sepsis, toxic shock syndrome and transplant rejection, and may also be treated in some embodiments.

Inflammatory conditions of the central nervous system can be treated in some embodiments of the instant invention, including, but not limited to, neurogenic inflammation and neurodegenerative diseases, such as Alzheimer's disease, and the like.

In some embodiments, compositions comprising combinations of compounds, forms and/or agents described herein provide treatments for autoimmune conditions. Autoimmune conditions as used herein can include organ or tissue-specific autoimmune conditions, as well as those which affect the whole body. Organ or tissue-specific autoimmune conditions that can be treated in some embodiments of the instant invention include, e.g., type I diabetes mellitus, multiple sclerosis, primary billiary cirrhosis, Hashimotos thyroiditis, pemicious anemia, Crohn's disease, Addison's disease, myasthenia gravis, rheumatoid arthritis, uveitis, psoriasis, Guillain-Barre Syndrome, Graves' disease, and the like. Systemic autoimmune conditions that can be treated in some embodiments include, e.g., systemic lupus erythematosus, ermatomyositis, and the like.

As detailed above, in some embodiments, combinations comprising a statin lactone and one or more additional active agents can be used in treating one or more of the inflammatory conditions provided herein. Preferred combinations can depend on the affected system. For example, combinations comprising a statin lactone and a salt form of a hydroxy acid statin are preferred in the treatment of inflammatory conditions of the vascular system and central nervous system, especially Alzheimer's disease, as well as inflammatory conditions of the skin, especially eczema, psoriasis, acne and the effects of ageing. Also as detailed above, preferred combinations comprise atorvastatin lactone with a salt of either atorvastatin or pitavastatin in a molar ratio of about 90:10 to about 10:90. In other embodiments, combinations comprising a statin lactone and a non-statin anti-inflammatory agent are preferred, e.g., where combinations comprising atorvastatin lactone and a non-steroidal anti-inflammatory drug are used in a molar ratio of about 90:10 to about 10:90.

One of the purposes of this invention is to teach combinations of compounds that can produce synergistic effects in treating an inflammatory condition, e.g., one or more of the inflammatory conditions provided herein. For example, in some preferred embodiments, a combination of a statin lactone and another active agent provides a synergistic effect in treating an inflammatory condition, as detailed above. Additional details of combinations providing synergistic inhibitory effects in treating inflammation are provided in Example 8 below.

### III. Formulations, Routes of Administration, and Effective Doses

Yet another aspect of the present invention relates to formulations, routes of administration and effective doses for pharmaceutical compositions comprising a compound or combination of compounds of the instant invention. Such pharmaceutical compositions can be used to treat inflammatory, MAP kinase-related, and/or HMG-CoA reductase-related conditions, as described in detail above.

The compounds described herein may be provided in a either the lactone or acid form, and/or may be allowed to interconvert in vivo after administration. That is, either the δ-lactone or hydroxy carboxylic acid form, or pharmaceutically acceptable salts, esters or amides thereof, may be used in developing a formulation for use in the present invention. Further, in some embodiments, the compound may be used in combination with one or more other compounds or with one or more other forms. For example a formulation may comprise both the lactone and acid forms in particular proportions, depending on the relative potencies of the lactone and acid forms and the intended indication. For example, in compositions for treating both MAP kinase- and HMG-CoA reductase-related conditions where the lactone form inhibits MAP kinase and the acid (carboxylate) form inhibits HMG-CoA reductase, and where potencies are similar, about a 1:1 ratio of lactone to acid forms may be used. The two forms may be formulated together, in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

The term “pharmaceutically acceptable salt” means those salts which retain the biological effectiveness and properties of the compounds used in the present invention, and which are not biologically or otherwise undesirable. For example, a pharmaceutically acceptable salt does not interfere with the beneficial effect of a compound of the invention in inhibiting MAP kinase and/or HMG-CoA reductase, e.g., in treating an inflamatory, MAP kinase-related and/or HMG-CoA reductase related condition.

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compound(s) contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide refers to those which retain biological effectiveness and properties of the compounds used in the present invention, and which are not biologically or otherwise undesirable. For example, the ester or amide does not interfere with the beneficial effect of a compound of the invention in inhibiting MAP kinase and/or HMG-CoA reductase, e.g., in treating an inflamatory, MAP kinase-related and/or HMG-CoA reductase related condition. Typical esters include ethyl, methyl, isobutyl, ethylene glycol, and the like. Typical amides include unsubstituted amides, alkyl amides, dialkyl amides, and the like.

In some embodiments, a compound may be administered in combination with one or more other compounds, forms, and/or agents, e.g., as described above. Pharmaceutical compositions comprising combinations of a statin lactone with one or more other active agents can be formulated to comprise certain molar ratios. For example, molar ratios of about 99:1 to about 1:99 of statin lactone to the other active agent can be used. Preferably, the range of molar ratios of statin lactone: other active agent is selected from about 80:20 to about 20:80; about 75:25 to about 25:75, about 70:30 to about 30:70, about 66:33 to about 33:66, about 60:40 to about 40:60; about 50:50; and about 90:10 to about 10:90. More preferably, the molar ratio of statin lactone:other active agent is about 1:9, and most preferably about 1:1. The two compounds, forms and/or agents may be formulated together, in the same dosage unit e.g. in one cream, suppository, tablet, capsule, or packet of powder to be dissolved in a beverage; or each compound, form, and/or agent may be formulated in separate units, e.g, two creams, suppositories, tablets, two capsules, a tablet and a liquid for dissolving the tablet, a packet of powder and a liquid for dissolving the powder, etc.

If necessary or desirable, the compounds and/or combinations of compounds may be administered with still other agents. The choice of agents that can be co-administered with the compounds and/or combinations of compounds of the instant invention can depend, at least in part, on the condition being treated. Agents of particular use in the formulations of the present invention include, for example, any agent having a therapeutic effect for kinase-related and/or HMG-CoA reductase-related conditions, including, e.g., drugs used to treat inflammatory conditions. For example, in treatments for acne, formulations of the instant invention may additionally contain one or more conventional acne treatments, such as keratolytic agents, e.g., retinoids, particularly retinoic acid; anti-inflammatory agents, such as peroxides, particularly benzoyl peroxide; and antiseborrhoeic agents. In treatments for osteoporosis, as another example, formulations may additionally contain one or more supplements, such as vitamin D and/or calcium, and/or one or more biphosphonate medications, e.g., which block bone resorption.

In still other embodiments, compounds and/or combinations of compounds described herein can be co-formulated and/or co-administered with agents useful for the prevention and/or treatment of atherosclerosis and its sequelae. These agents include, but are not limited to, e.g., inhibitors of cholesterol ester transferase protein (CETP) (e.g., JTT-705, torcetrapib); inhibitors of sterol acyl-CoA-acyl transferase (ACAT) (e.g., pactimibe, SMP-797, K-604); inhibitors of microsomal triglyceride transferase protein (MTTP) (e.g., implitipide, JTT-130); modulators of peroxisome proliferators activated receptors (PPARs) (e.g., binifibrate, gemfibrozil, clinofibrate, ronifibrate, fenofibrate, bezafibrate, LY-929, GW-516, GW-590735, NS-220, LY-674, DRF-10945, SB-641597, AVE-8134, AVE-0847, ciglitazone, pioglitazone, darglitazone, rosiglitazone, isaglitazone, reglitazar, farglitazar, tesaglitazar, balaglitazone, ragaglitazar, rivoglitazone, imiglitazar, edaglitazone, oxeglitazar, muraglitazar); inhibitors of cholesterol absorption (e.g., ezetimibe, colesevelam hydrochloride, cholestyramine, colestimide, colestipol hydrochloride, BTG-511); vitamins (e.g., niacin); inhibitors of platelet aggregation (e.g., aspirin, clopidogrel, D-003); inhibitors of ileal bile acid transport (IBAT) (e.g., S-8921, BARI-1741); inhibitors of lipoprotein-associated phospholipase A2 (Lp-PLA2) (e.g., SB-480848, SB-659032, SB-677116); inhibitors of squalene synthase (e.g., TAK-475); antagonists of chemokine CCR2 receptor (e.g., INCB-3284, C-8834, C-1602).

The compound(s) (or pharmaceutically acceptable salts, esters or amides thereof) may be administered per se or in the form of a pharmaceutical composition wherein the active compound(s) is in an admixture or mixture with one or more pharmaceutically acceptable carriers. A pharmaceutical composition, as used herein, may be any composition prepared for administration to a subject. Pharmaceutical compositions for use in accordance with the present invention may be formulated in conventional manner using one or more physiologically acceptable carriers, comprising excipients, diluents, and/or auxiliaries, e.g., which facilitate processing of the active compounds into preparations that can be administered. Proper formulation may depend at least in part upon the route of administration chosen. The compound(s) useful in the present invention, or pharmaceutically acceptable salts, esters, or amides thereof, can be delivered to a patient using a number of routes or modes of administration, including oral, buccal, topical, rectal, transdermal, transmucosal, subcutaneous, intravenous, and intramuscular applications, as well as by inhalation.

For oral administration, the compounds can be formulated readily by combining the active compound(s) with pharmaceutically acceptable carriers well known in the art. Such carriers enable the compounds of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the compounds of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use may contain compound(s) of this invention with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the compounds into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, for example, Bangham et al., J. Mol. Biol. 23: 238-252 (1965) and Szoka et al., Proc. Natl. Acad. Sci. USA 75: 4194-4198 (1978), incorporated herein by reference. Ligands may also be attached to the liposomes to direct these compositions to particular sites of action. Compounds of this invention may also be integrated into foodstuffs, e.g, cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The compounds may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

In some preferred embodiments, oral formulations are used to treat Alzheimer's and/or other inflammatory conditions of the central nervous system. In some preferred embodiments, oral formulations are used to treat arthritis, rheumatoid arthritis and/or other inflammatory conditions of the musculoskeletal system. As detailed above, preferred compositions in such embodiments are those comprising a statin lactone and a non-statin anti-inflammatory agent.

In some preferred embodiments, oral formulations are used to treat inflammatory conditions of the vascular system especially hypercholesterolemia, hyperlipidemia, atherosclerosis, peripheral occlusive disease, myocardial infarction, and stroke. Preferred compositions in such embodiments are those comprising a statin lactone and a salt form of a hydroxy acid statin. More preferred are combinations of atorvastatin lactone with a salt of either atorvastatin or pitavastatin, even more preferably in a molar ratio of about 90:10 to about 10:90. Additional details of such preferred embodiments for oral formulations are provided in Example 4, as outlined above.

For injection, the compounds of the present invention may be formulated in aqueous solutions, preferably in physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P.

In addition to the formulations described previously, the compounds may also be formulated as a depot preparation. Such long acting formulations may be administered by implantation or transcutaneous delivery (for example subcutaneously or intramuscularly), intramuscular injection or use of a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In some embodiments, pharmaceutical compositions comprising one or more compounds of the present invention exert local and regional anti-inflammatory effects when administered topically or injected at or near particular sites of inflammation. Direct topical application, e.g., of a viscous liquid, gel, jelly, cream, lotion, ointment, suppository, foam, or aerosol spray, may be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In some preferred embodiments, local/topical formulations comprising a statin lactone and a non-statin anti-inflammatory agent are used to treat inflammatory conditions. Preferred compositions in such embodiments are those comprising atorvastatin lactone and a non-steroidal anti-inflammatory drug, even more preferably in a molar ratio of about 90:10 to about 10:90.

In some preferred embodiments, local/topical formulations are used to treat allergic, inflammatory and/or autoimmune conditions of the skin or skin structures, especially for treating eczema, psoriasis, acne and the effects of aging. For example, for treating inflammatory and/or autoimmune conditions, a cream comprising a compound of the invention may be topically applied to the affected site, for example, sites displaying red plaques or dry scales in psoriasis, or areas of irritation and dryness in dermatitis. Preferred compositions in such embodiments are those comprising a statin lactone and a salt form of a hydroxy acid statin. More preferred are combinations of atorvastatin lactone with a salt of either atorvastatin or pitavastatin, even more preferably in a molar ratio of about 90:10 to about 10:90.

Pharmaceutical compositions of the present invention may contain a cosmetically or dermatologically acceptable carrier. Such carriers are compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, a compound or combination of compounds of the instant invention may be formulated in an oleaginous hydrocarbon base, an anhydrous absorption base, a water-in-oil absorption base, an oil-in-water water-removable base and/or a water-soluble base.

The compositions according to the present invention may be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. These compositions can be prepared according to conventional methods. Other than the compounds of the invention, the amounts of the various constituents of the compositions according to the invention are those conventionally used in the art. These compositions in particular constitute protection, treatment or care creams, milks, lotions, gels or foams for the face, for the hands, for the body and/or for the mucous membranes, or for cleansing the skin. The compositions may also consist of solid preparations constituting soaps or cleansing bars.

Compositions of the present invention may also contain adjuvants common to the cosmetic and dermatological fields, such as hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants are those conventionally used in the fields considered and, for example, are from about 0.01% to about 20% of the total weight of the composition. Depending on their nature, these adjuvants may be introduced into the fatty phase, into the aqueous phase and/or into the lipid vesicles.

In some preferred embodiments, the compounds of the present invention are delivered in soluble rather than suspension form, which allows for more rapid and quantitative absorption to the sites of action. In general, formulations such as jellies, creams, lotions, suppositories and ointments can provide an area with more extended exposure to the compounds of the present invention, while formulations in solution, e.g., sprays, provide more immediate, short-term exposure.

In some embodiments relating to topical/local application, the pharmaceutical compositions can include one or more penetration enhancers. For example, the formulations may comprise suitable solid or gel phase carriers or excipients that increase penetration or help delivery of compounds or combinations of compounds of the invention across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, α-bisabolol, d-limonene, urea and N,N-diethyl-m-toluamide, and the like Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In some embodiments, the pharmaceutical compositions will include one or more such penetration enhancers.

In some embodiments, the pharmaceutical compositions for local/topical application can include one or more antimicrobial preservatives such as quaternary ammonium compounds, organic mercurials, p-hydroxy benzoates, aromatic alcohols, chlorobutanol, and the like.

Gastrointestinal allergic, inflammatory and/or autoimmune conditions can be effectively treated with orally- or rectally delivered solutions, suspensions, ointments, enemas and/or suppositories comprising a compound or combination of compounds of the present invention. Local/topical formulations are preferred for therapy of Crohn's colitis and other allergic, inflammatory and/or autoimmune diseases of the gastrointestinal system.

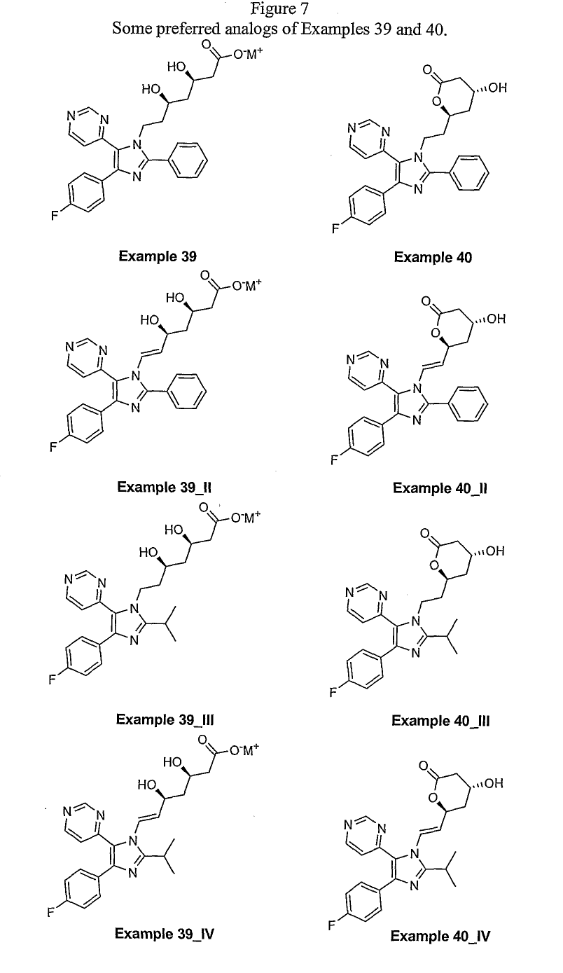

In treating inflammatory bowel disease, for example, a suppository formulation of a compound or combination of compounds disclosed herein can be used. In such embodiments, the active ingredient can produce a benefit locally at or near the site of application, rather than systemically, by inhibiting a MAP kinase inhibitor, e.g., p38αMAP kinase. In some preferred embodiments, a lactone form (formula I) of a known statin is used in formulations for topical inhibition of MAP kinase. In more preferred embodiments, the statin lactone used topically is a synthetic statin lactone, such as atorvastatin, cerivastatin, fluvastatin, pitavastatin, glenvastatin, and/or rosuvastatin, including, for example, structures provided in FIGS. 7 and 8. In some preferred embodiments, compounds modified to favor a closed ring structure, such as formulas III and IV of FIG. 9, are used in formulations for topical inhibition of MAP kinase. In more preferred embodiments, the modified compound is derived from a synthetic statin lactone, such as atorvastatin, cerivastatin, fluvastatin, pitavastatin, glenvastatin, and/or rosuvastatin.

Respiratory allergic, inflammatory and/or autoimmune conditions can be effectively treated with aerosol solutions, suspensions or dry powders comprising a compound or combination of compounds of the present invention. Administration by inhalation is particularly useful in treating inflammatory conditions of the lung. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. For example, an aerosol formulation comprising a MAP kinase and/or HMG CoA reductase inhibitor can be dissolved, suspended or emulsified in a propellant or a mixture of solvent and propellant, e.g., for administration as a nasal spray or inhalant. Aerosol formulations may contain any acceptable propellant under pressure, preferably a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the compound or combination of compounds of the present invention is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the compound or combination of compounds in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants useful in the present invention include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as the ethers. An aerosol formulation of the invention can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present invention can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of a compound of the invention such as a novel HMG-CoA reductase inhibitor in (substantially) pure propellant or as a mixture of propellant and solvent. The solvent can be used to dissolve the compound and/or retard the evaporation of the propellant. Solvents useful in the invention include, for example, water, ethanol and glycols. Any combination of suitable solvents can be use, optionally combined with preservatives, antioxidants, and/or other aerosol components.

An aerosol formulation can also be a dispersion or suspension. A suspension aerosol formulation may comprise a suspension of a compound or combination of compounds of the instant invention, e.g., an HMG CoA reductase inhibitor, and a dispersing agent. Dispersing agents useful in the invention include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as a compound or combination of compounds of the invention, e.g., an HMG-CoA reductase inhibitor. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

Pharmaceutical compositions suitable for use in the present invention include compositions wherein the active ingredients are present in an effective amount, i.e., in an amount effective to achieve therapeutic and/or prophylactic benefit in at least one of a MAP kinase-related condition and an HMG-CoA reductase-related condition. The actual amount effective for a particular application will depend on the condition or conditions being treated, the condition of the subject, the formulation, and the route of administration, as well as other factors known to those of skill in the art. Determination of an effective amount of a MAP kinase and/or HMG-CoA reductase inhibitor is well within the capabilities of those skilled in the art, in light of the disclosure herein, and will be determined using routine optimization techniques.

The effective amount for use in humans can be determined from animal models. For example, a dose for humans can be formulated to achieve circulating, liver, topical and/or gastrointestinal concentrations that have been found to be effective in animals. One skilled in the art can determine the effective amount for human use, especially in light of the animal model experimental data described herein. Example 8, provided below, details an experiment where an inflammatory condition was induced in mice, compositions of the present invention were administered, and anti-inflammatory effect observed. As described in more detail below, Table II shows the resulting percentage of inhibition and effective amounts of compounds and combinations of compounds of the instant invention. Based on this animal data, and other types of similar data, those skilled in the art can determine the effective amounts of compositions of the present invention appropriate for humans.

The effective amount when referring to a compound or combination of compounds of the invention will generally mean the dose ranges, modes of administration, formulations, etc., that have been recommended or approved by any of the various regulatory or advisory organizations in the medical or pharmaceutical arts (e.g., FDA, AMA) or by the manufacturer or supplier. Effective amounts of HMG-CoA reductase inhibitors can be found, for example, in the Physicians Desk Reference. For example, daily doses for atorvastatin calcium range from about 2 mg to about 50 mg, from about 3 mg to about 30 mg, typically about 10 mg. A daily dose for cerivastatin sodium is about 200 μg, while daily doses for fluvastatin sodium, rosuvastatin sodium, pravastatin sodium and simvastatin are each about 20 mg. Some preferred compounds of this invention, e.g., analogs of HMG-CoA reductase inhibitors, may be useful in about the same dosages, or less than or more than dosages typical of known HMG-CoA reductase inhibitors.

Effective amounts of MAP kinase inhibitors can be found, for example, in published reports of the results of human clinical trials. Generally, the recommended dosage for a MAP kinase inhibitor of the present invention, e.g., a p38αMAP kinase inhibitor, is a dose of about 0.01 mg/kg to about 1,000 mg/kg, more preferably from about 0.1 mg/kg to about 20 mg/kg on a daily basis, provided orally. The inhibitor is typically administered in a dose of about 100 mg, which is in the range of doses that will be useful in the present invention. Using other routes of administration, it is believed that a dose of about 0.01 mg/kg/day to about 1,000 mg/kg/day of a MAP kinase inhibitor will be used; preferably a dose between about 0.1 mg/kg/day and about 20 mg/kg/day will be used.

Generally, the recommended dosage for an HMG-CoA reductase inhibitor of the present invention is a dose of about 0.01 mg/kg to about 1,000 mg/kg, more preferably from about 0.1 mg/kg to about 20 mg/kg on a daily basis, provided orally. The inhibitor is typically administered in a dose of about 10 mg, which is in the range of doses that will be useful in the present invention. Using other routes of administration, it is believed that a dose of about 0.01 mg/kg/day to about 1,000 mg/kg/day of an HMG-CoA reductase inhibitor will be used; preferably a dose between about 0.1 mg/kg/day and about 1 mg/kg/day will be used.

Further, appropriate doses for a statin lactone, hydroxy acid form of a statin or non-statin anti-inflammatory agent can be determined based on in vitro experimental results provided herein. For example, the in vitro potency of a compound in inhibiting HMG-CoA reductase and/or inhibiting inflammation mediators provides information useful in the development of effective in vivo dosages to achieve similar biological effects.

Effective amounts of compounds and/or combinations of compounds of the instant invention for use in increasing HDL levels can similarly be determined based on in vitro experimental data, including animal model data. For example, an animal model can be used to determine a percentage increase in HDL levels that would be desirable in humans, and corresponding effective doses of the compound(s) or combinations of compounds to achieve such levels.

In some embodiments, administration of compounds of the present invention may be intermittent, for example administration once every two days, every three days, every five days, once a week, once or twice a month, and the like. In some embodiments, the amount, forms, and/or amounts of the different forms may be varied at different times of administration. For example, at one point in time, the acid form of a compound of the present invention may be administered, while at another time the corresponding lactone form may be used.

A person of skill in the art would be able to monitor in a patient the effect of administration of a particular compound. For example, cholesterol levels can be determined by measuring LDL, HDL, and/or total serum cholesterol levels. The release of pro-inflammatory cytokines can be determined by measuring TNF-α and/or IL-1β. Other techniques would be apparent to one of skill in the art.

All publications, patents, and patent applications mentioned in this specification are herein incorporated by reference to the same extent as if each individual publication, patent, or patent application was specifically and individually indicated to be incorporated by reference.

### EXAMPLES

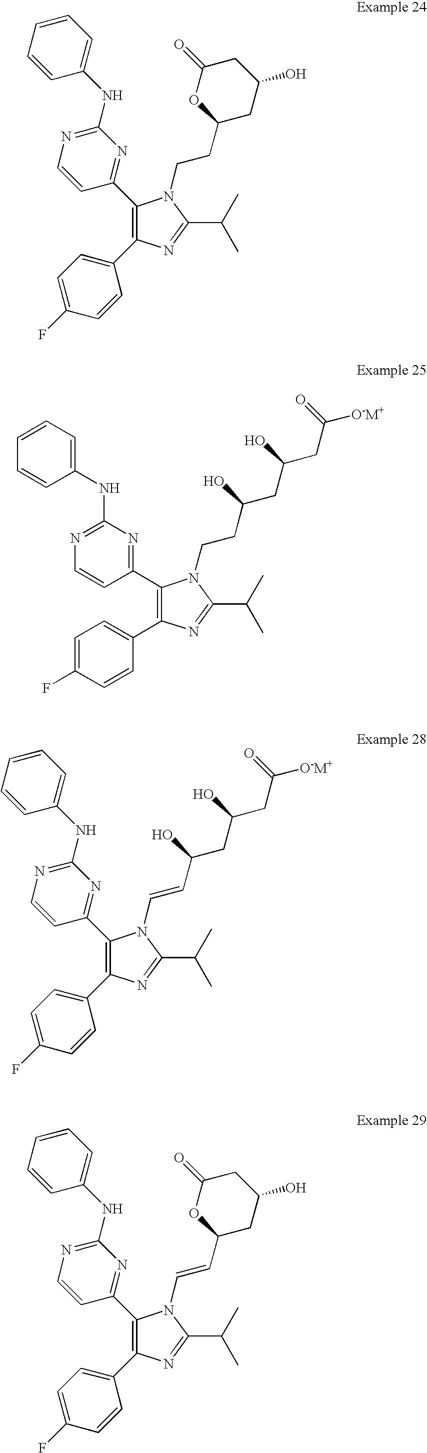

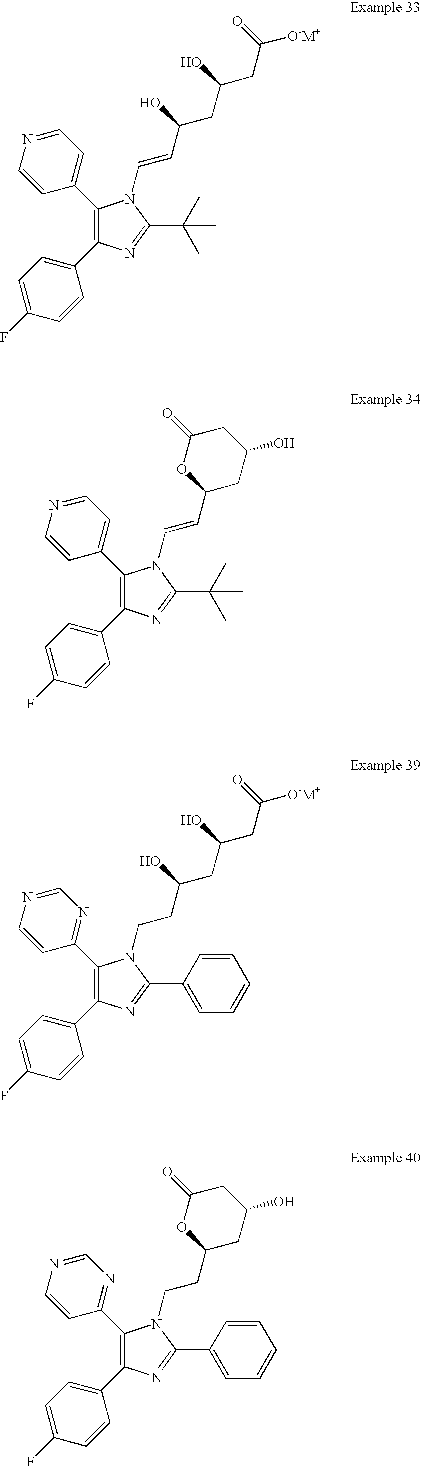

### Example 24

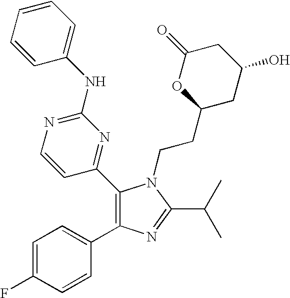

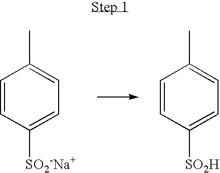

### Para-toluenesulfinic acid

To a solution of para-toluenesulfinic acid sodium salt (200.0 g) in water (750 mL) and tert-butyl methyl ether was added HCl (conc, 93.3 mL). The mixture was left to stir for 1 hour then the layers separated. The organic layer was dried (Na₂SO₄), filtered and concentrated to afford the free acid (126.0 g).

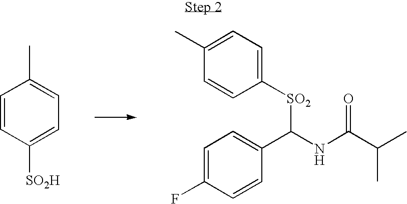

### N-((4-fluorophenyl)(tosyl)methyl)isobutyramide

A flask was charged with isobutyramide (283.1 g), acetonitrile (1.3 L), toluene (1.3 L), trimethylsilyl chloride (305 mL) and 4-fluorobenzaldehyde (232 mL). The mixture was heated to 50° C. with stirring for 4 hours forming a thick precipitate. Powdered para-toluene sulfinic acid (508 g) was added portionwise and the resulting mixture heated at 45° C. for 16 hours. On cooling, the mixture was partitioned between water and tert-butyl methyl ether, stirred for 10 minutes then filtered to afford the title compound which was dried under vacuum (303 g).

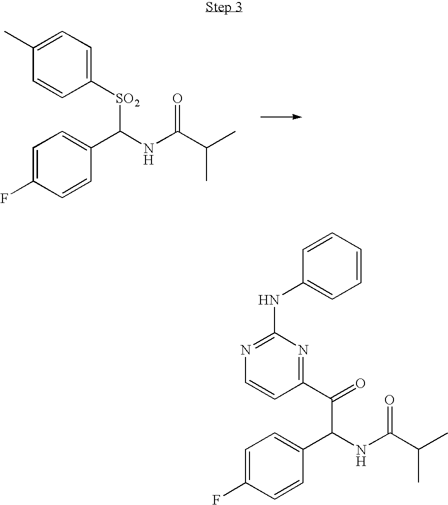

### N-(1-(4-fluorophenyl)-2-oxo-2-(2-(phenylamino)pyrimidin-4-yl)ethyl)isobutyramide

A mixture of N-((4-fluorophenyl)(tosyl)methyl)isobutyramide (25.0 g) and 3-methyl-5-(2-hydroxyethyl)-4-methylthiazolium iodide (3.06 g) was treated with dichloromethane (400 mL) and 2-(phenylamino)pyrimidine-4-carboxaldehyde (15.68 g). The mixture was purged with nitrogen, then heated to 40° C. and triethylamine (151 mL) added in one portion. After heating at 40° C. for 2 hours, the mixture was cooled to room temperature and stirred for 16 hours then the solvent removed in vacuo. The crude residue was purified by passing through a plug of silica gel to furnish the desired compound (23.9 g).

LC/MS: C₂₂H₂₁FN₄O₂ requires 392.2; seen M/Z 393.2, [M+H]⁺, 391.3 [M−H]⁻. Retention time 3.37 min.

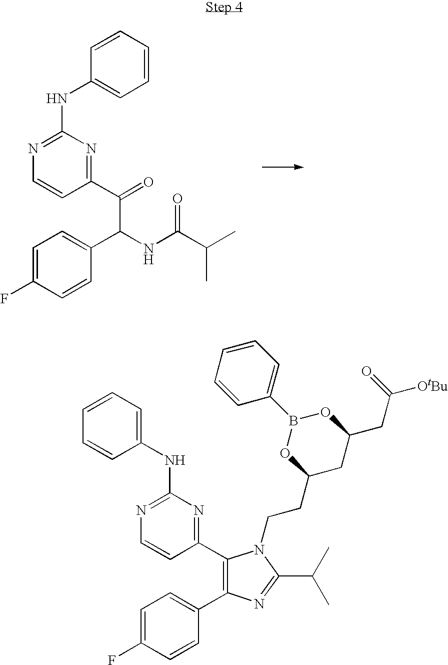

### tert-butyl 2-((4R,6R)-6-(2-(4-(4-fluorophenyl)-2-isopropyl-5-(2-(phenylamino)pyrimidin-4-yl)-1H-imidazol-1-yl)ethyl)-2-phenyl-1,3,2-dioxaborinan-4-yl)acetate

A mixture of N-(1-(4-fluorophenyl)-2-oxo-2-(2-(phenylamino)pyrimidin-4-yl)ethyl)isobutyramide (1.00 g), tert-butyl 2-((4R,6R)-6-(2-aminoethyl)-2-phenyl-1,3,2-dioxaborinan-4-yl)acetate, (4.07 g), pivalic acid (1.64 g) and methyltetrahydrofuran (8 mL) were heated together in a microwave at 300 W for 1 hour at 150° C. The mixture was then partitioned between ethyl acetate and aqueous sodium hydrogencarbonate. The aqueous layer was extracted with further ethyl acetate and the combined organics dried (MgSO₄), filtered and concentrated. The resulting title compound was used crude in the next step.

LC/MS: C₃₉H₄₃BFN₅O₄ requires 675.3; seen M/Z 590.4, [M-C₆H₃B+H]⁺, 588.6 [M-C₆H₃B—H]⁻. Retention time 3.72 min

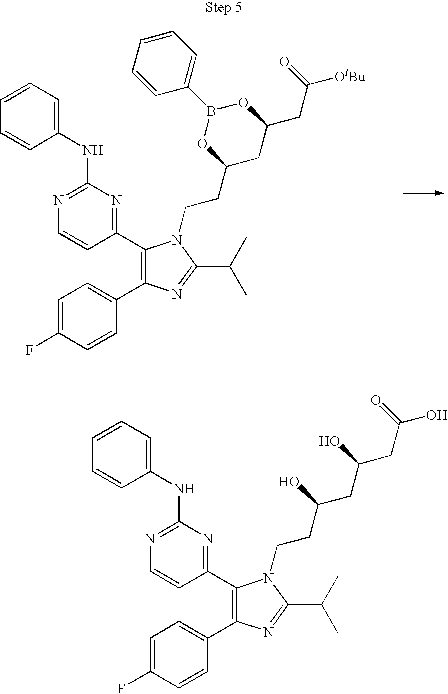

### (3R,5R)-7-(4-(4-fluorophenyl)-2-isopropyl-5-(2-(phenylamino)pyrimidin-4-yl)-1H-imidazol-1-yl)-3,5-dihydroxyheptanoic acid

To a stirred solution of aqueous sodium hydroxide (1M, 19.14 mL) was added aqueous hydrogen peroxide (35% wt, 1.91 mL). The mixture was left to stir at room temperature for 30 minutes. To this was added a solution of tert-butyl 2-((4R,6R)-6-(2-(4-(4-fluorophenyl)-2-isopropyl-5-(2-(phenylamino)pyrimidin-4-yl)-1H-imidazol-1-yl)ethyl)-2-phenyl-1,3,2-dioxaborinan-4-yl)acetate (4.31 g) in tetrahydrofuran (90 mL) and the mixture left to stir at room temperature for 2 hours. A solution of sodium hydroxide (1M, 76.5 mL) was added and the mixture stirred at room temperature for 1 hour. The tetrahydrofuran was removed in vacuo and the residue partitioned between water (at pH 10) and ethyl acetate. The aqueous layer was extracted twice more with ethyl acetate, then acidified to pH 5 with 1M HCl and extracted with ethyl acetate. The organic extract at pH 5 was dried (MgSO₄), filtered and concentrated to afford the desired compound which was used crude in the next step.

LCMS: C₂₉H₃₂FN₅O₄ requires 533.2; seen M/Z 534.3, [M+H]⁺, 532.4 [M−H]⁻. Retention time 1.77 min.

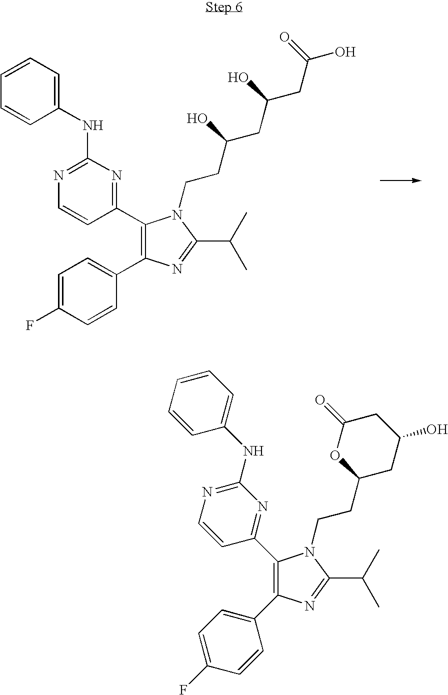

### R,6R)-6-(2-(4-(4-fluorophenyl)-2-isopropyl-5-(2-(phenylamino)pyrimidin-4-yl-1H-imidazo-1-yl)ethyl)-4-hydroxytetrahydro-2H-pyran-2-one

Crude (3R,5R)-7-(4-(4-fluorophenyl)-2-isopropyl-5-(2-(phenylamino)pyrimidin-4-yl)-1H-imidazol-1-yl)-3,5-dihydroxyheptanoic acid (2.09 g) was dissolved in acetonitrile (50 mL) and heated to 60° C. for 3 hours. On cooling, the mixture was partitioned between dichloromethane and aqueous sodium hydrogencarbonate. The aqueous was extracted with further dichloromethane and the combined organics dried (MgSO₄), filtered and concentrated. Following purification by flash chromatography the title compound was obtained (1.05 g).

LCMS: C₂₉H₃₀FN₅O₃ requires 515.2; seen M/Z 516.5, [M+H]⁺, 514.5 [M−H]⁻. Retention time 3.00 min.

¹H-NMR (400 MHz, CDCl₃, δ)—1.40 (d, 3H), 1.42 (d, 3H), 1.48-1.56 (m, 2H), 1.90-1.98 (m, 2H), 2.50-2.64 (m, 2H), 3.12 (sept, 1H), 4.20-4.26 (m, 1H), 4.29-4.39 (m, 1H), 4.52-4.62 (m, 2H), 6.51 (d, 1H), 7.02 (t, 2H), 7.07 (t, 1H), 7.29 (s, 1H), 7.35 (t, 2H), 7.45-7.50 (m, 2H), 7.62 (d, 2H), 8.21 (d, 1H).

### Example 25

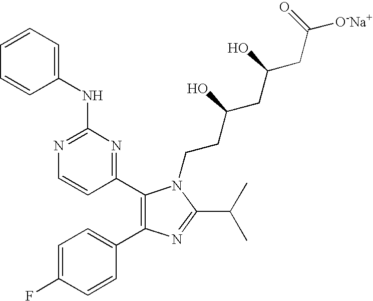

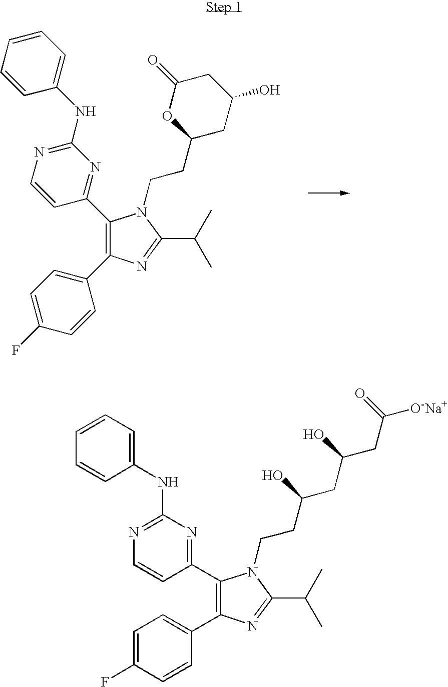

### (3R,5R)-7-(4-(4-fluorophenyl)-2-isopropyl-5-(2-(phenylamino)pyrimidin-4-yl)-1H-imidazol-1-yl)-3,5-dihydroxyheptanoic acid sodium salt

(4R,6R)-6-(2-(4-(4-fluorophenyl)-2-isopropyl-5-(2-(phenylamino)pyrimidin-4-yl)-1H-imidazol-1-yl)ethyl)-4-hydroxytetrahydro-2H-pyran-2-one (0.932 g) was dissolved in ethanol (40 mL) and aqueous sodium hydroxide (1M, 1.81 mL) added dropwise. The mixture was stirred at room temperature for 1 hour after which time the solvent was removed in vacuo to afford the title compound (1.00 g).

LCMS: C₂₉H₃₂FN₅O₄ requires 533.2; seen M/Z 534.3, [M+H]⁺, 532.4 [M−H]⁻. Retention time 1.77 min.

¹H-NMR (400 MHz, DMSO-d₆, δ)—1.15-1.23 (m, 1H), 1.30 (d, 3H), 1.32 (d, 3H), 1.34-1.43 (m, 1H), 1.46-1.56 (m, 1H), 1.64-1.74 (m, 1H), 1.76 (dd, 1H), 1.94 (dd, 2H), 3.20 (sept, 1H), 3.40-3.47 (m, 1H), 3.55-3.69 (m, 2H), 4.08-4.15 (m, 1H), 4.27-4.37 (m, 2H), 4.77-5.01 (brm, 1H), 6.63 (d, 1H), 6.94 (t, 1H), 7.13 (t, 2H), 7.25 (t, 2H), 7.41-7.46 (m, 2H), 7.68 (d, 2H), 8.39 (d, 1H).

### Example 28

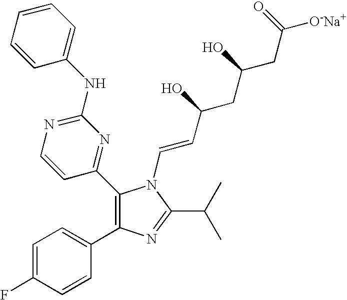

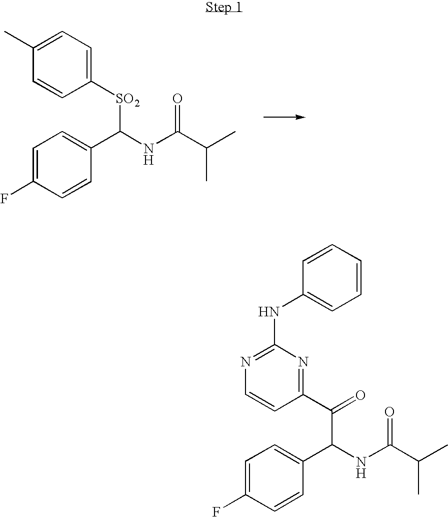

### N-(1-(4-fluorophenyl)-2-oxo-2-(2-(phenylamino)pyrimidin-4-yl)ethyl)isobutyramide

To a mixture of N-((4-fluorophenyl)(tosyl)methyl)isobutyramide (224.56 g) and 3-methyl-5-(2-hydroxyethyl)-4-methylthiazolium iodide (27.50 g) in dichloromethane (3 L) was added 2-(phenylamino)pyrimidine-4-carboxaldehyde (134.35 g) and the mixture purged with nitrogen then heated to 40° C. with stirring. Triethylamine (1.35 L) was then added in one portion and the mixture left at 40° C. for 4 hours, then at room temperature overnight. The solvents were removed in vacuo to furnish crude product which was used without purification in the next step.

LCMS: C₂₂H₂₁FN₄O₂ requires 392.2; seen M/Z 393.2, [M+H]⁺, 391.2 [M−H]⁻. Retention time 3.32 min.

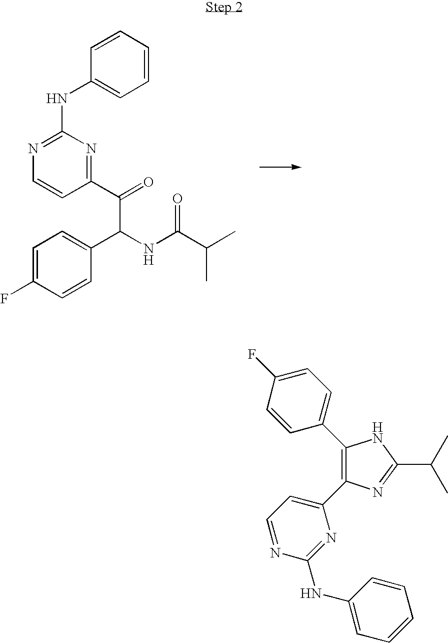

### 4-(5-(4-fluorophenyl)-2-isopropyl-1H-imidazol-4-yl)-N-phenylpyrimidin-2-amine

A mixture of N-(1-(4-fluorophenyl)-2-oxo-2-(2-(phenylamino)pyrimidin-4-yl)ethyl)isobutyramide and ammonium acetate (743 g) were dissolved in ethanol (2 L) and the mixture heated to 90° C. for 16 hours. On cooling, the mixture was concentrated in vacuo then partitioned between ethyl acetate and water. The organic layer was dried (MgSO₄), filtered and concentrated. The crude residue was purified by flash chromatography to furnish the desired compound (197 g).

LCMS: C₂₂H₂₀FN₅ requires 373.2; seen M/Z 374.3, [M+H]⁺, 372.3 [M−H]⁻. Retention time 3.20 min.

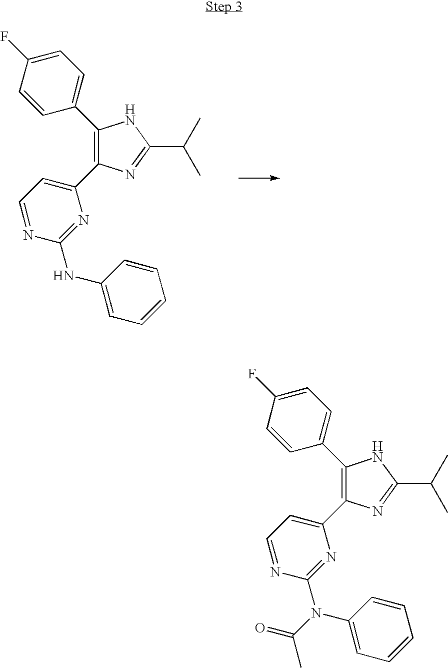

### N-(4-(5-(4-fluorophenyl)-2-isopropyl-1H-imidazol-4-yl)pyrimidin-2-yl)-N-phenylacetamide

A suspension of 4-(5-(4-fluorophenyl)-2-isopropyl-1H-imidazol-4-yl)-N-phenylpyrimidin-2-amine (50.0 g) in acetyl chloride (250 mL) was heated to reflux for 1 week. On cooling, the reaction mixture was neutralised carefully with aqueous sodium hydrogencarbonate then extracted with ethyl acetate. The organic layer was dried (MgSO₄), filtered and concentrated to furnish the title compound (50.22 g) which was used without further purification.

LCMS: C₂₄H₂₂FN₅O requires 415.2; seen M/Z 416.4, [M+H]⁺, 414.5 [M−H]⁻. Retention time 2.80 min.

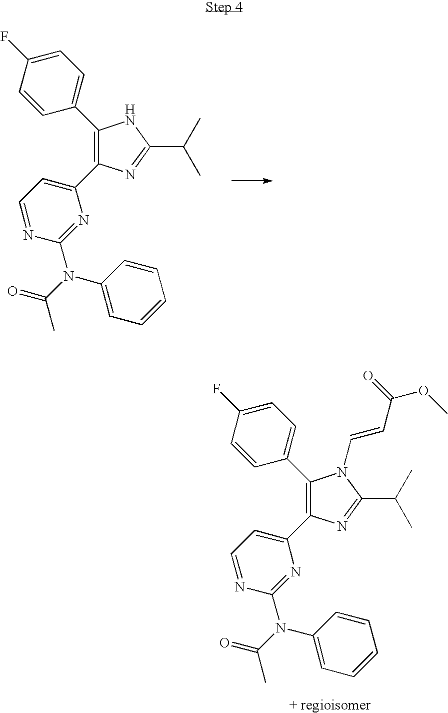

### (E)-methyl 3-(5-(4-fluorophenyl)-2-isopropyl-4-(2-(N-phenylacetamido)pyrimidin-4-yl)-1H-imidazol-1-yl)acrylate

A mixture of N-(4-(5-(4-fluorophenyl)-2-isopropyl-1H-imidazol-4-yl)pyrimidin-2-yl)-N-phenylacetamide (12.50 g) and methyl propiolate (12.96 g) were heated together in a microwave at 300 W for 2 hours 30 minutes at 100° C. The mixture was then transferred to a flask with ethyl acetate and all solvents removed in vacuo. The crude residue was used without further purification in the next step.

LCMS: C₂₈H₂₆FN₅O₃ requires 499.2; seen M/Z 500.3, [M+H]⁺. Retention time 3.45 min.

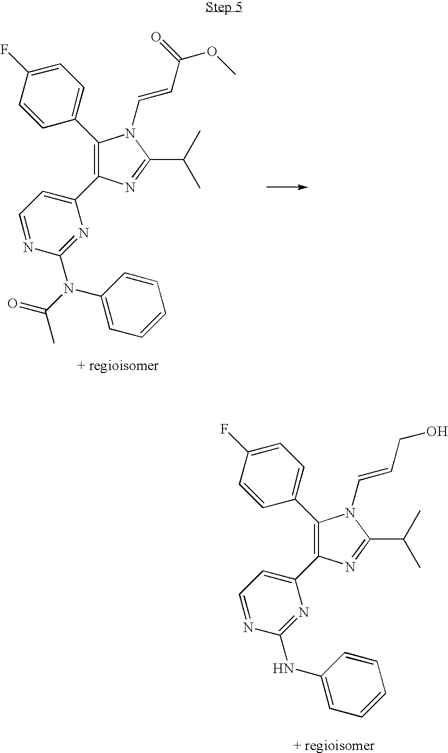

### (E)-3-(5-(4-fluorophenyl)-2-isopropyl-4-(2-(phenylamino)pyrimidin-4-yl)-1H-imidazol-1-yl)prop-2-en-1-ol

To a pre-dried flask under nitrogen was added a solution of (E)-methyl 3-(5-(4-fluorophenyl)-2-isopropyl-4-(2-(N-phenylacetamido)pyrimidin-4-yl)-1H-imidazol-1-yl)acrylate and its regioisomer (15.35 g) in tetrahydrofuran (12 mL). Nitrogen was bubbled through the solution for 10 minutes then the mixture cooled to −78° C. and DIBAL-H (1M solution in toluene, 93 mL) added dropwise. Further DIBAL-H (1M solution in toluene, 93 mL) was added after stirring at −78° C. for 2 hours. The reaction mixture was left to warm to room temperature over 16 hours. Aqueous ammonium chloride solution was added cautiously and after 10 minutes the mixture was extracted with dichloromethane several times. The combined organic washes were dried (MgSO₄), filtered and concentrated to afford the desired compound as a mixture of regioisomers which were used without further purification in the next step.

LCMS: C₂₅H₂₄FN₅O requires 429.2; seen M/Z 430.4, [M+H]⁺, 428.5 [M−H]⁻. Retention time 3.05 min.

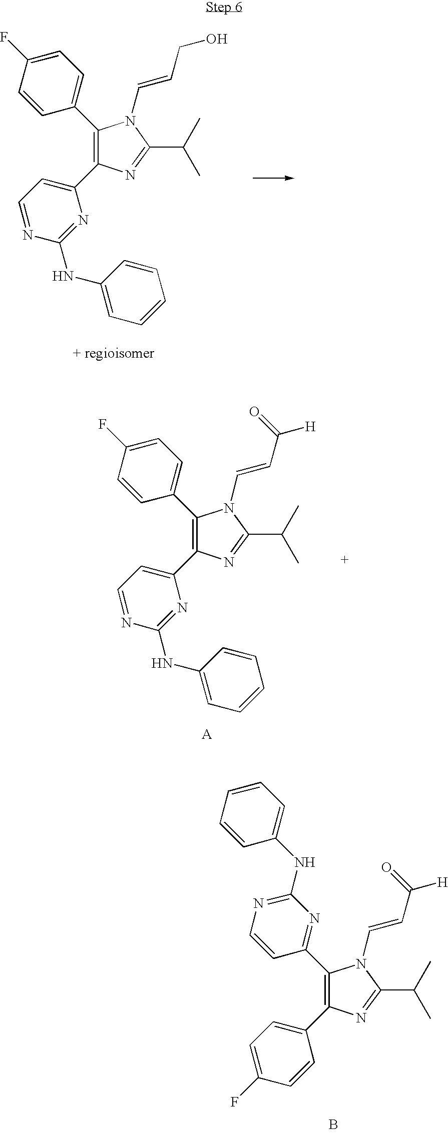

### (E)-3-(5-(4-fluorophenyl)-2-isopropyl-4-(2-(phenylamino)pyrimidin-4-yl)-1H-imidazol-1-yl)acrylaldehyde (A) and (E)-3-(4-(4-fluorophenyl)-2-isopropyl-5-(2-(phenylamino)pyrimidin-4-yl)-1H-imidazol-1-yl)acrylaldehyde (B)

To a solution of (E)-3-(5-(4-fluorophenyl)-2-isopropyl-4-(2-(phenylamino)pyrimidin-4-yl)-1H-imidazol-1-yl)prop-2-en-1-ol and its regioisomer (13.2 g) in dichloromethane (500 mL) was added manganese dioxide (130 g) and the mixture stirred gently at room temperature for 24 hours. After filtration through celite, the reaction mixture was concentrated in vacuo. The residue was purified by flash chromatography to furnish (E)-3-(5-(4-fluorophenyl)-2-isopropyl-4-(2-(phenylamino)pyrimidin-4-yl)-1H-imidazol-1-yl)acrylaldehyde (A, 1.08 g) and (E)-3-(4-(4-fluorophenyl)-2-isopropyl-5-(2-(phenylamino)pyrimidin-4-yl)-1H-imidazol-1-yl)acrylaldehyde (B, 1.40 g).

A) LCMS: C₂₅H₂₂FN₅O requires 427.2; seen M/Z 428.4, [M+H]⁺, 428.5 [M−H]⁻. Retention time 3.57 min.

¹H-NMR (400 MHz, CDCl₃, δ)—1.49 (d, 6H), 3.25 (sept, 1H), 5.74 (dd, 1H, J=14.8, 7.2 Hz), 6.87 (s, 1H), 6.96 (t, 1H), 7.13-7.24 (m, 7H), 7.36-7.40 (m, 2H), 7.45 (d, 1H, J=14.8 Hz), 8.35 (d, 1H), 9.43 (d, 1H, J=7.2 Hz).

B) LCMS: C₂₅H₂₂FN₅O requires 427.2; seen M/Z 428.4, [M+H]⁺, 428.5 [M−H]⁻. Retention time 3.67 min.

¹H-NMR (400 MHz, CDCl₃, δ)—1.46 (d, 6H), 3.24 (sept, 1H), 6.10 (dd, 1H, J=14.4, 7.6 Hz), 6.63 (d, 1H), 7.01-7.11 (m, 3H), 7.21 (s, 1H), 7.31 (t, 2H), 7.49-7.56 (m, 4H), 8.01 (d, 1H, J=14.4 Hz), 8.31 (d, 1H), 9.33 (d, 1H, J=7.6 Hz).

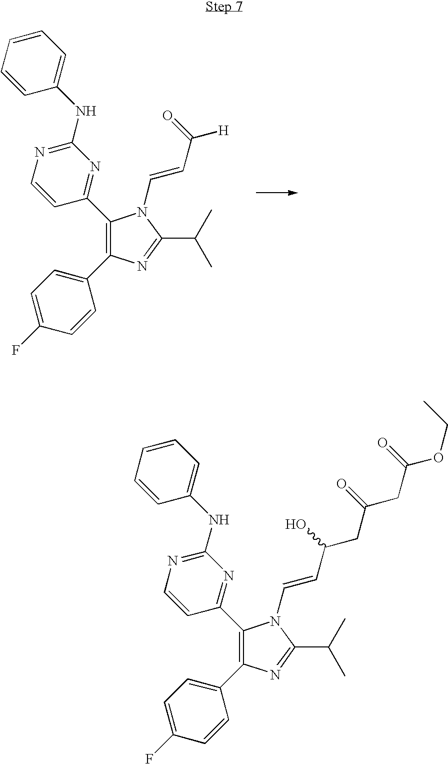

### (S and R,E)-ethyl 7-(4-(4-fluorophenyl)-2-isopropyl-5-(2-(phenylamino)pyrimidin-4-yl)-1H-imidazol-1-yl)-5-hydroxy-3-oxohept-6-enoate

A flask containing sodium hydride (0.935 g) and tetrahydrofuran (12 mL) was cooled to −10° C. under nitrogen. Ethyl acetoacetate (0.305 g) in tetrahydrofuran (1 mL) was added dropwise and the mixture stirred at −10° C. for 10 minutes. Butyllithium (2.5 M in hexanes, 1.4 mL) was then added dropwise. The flask was allowed to warm to 0° C. over 30 minutes then re-cooled to −10° C. whereupon a solution of (E)-3-(4-(4-fluorophenyl)-2-isopropyl-5-(2-(phenylamino)pyrimidin-4-yl)-1H-imidazol-1-yl)acrylaldehyde (0.503 g) in tetrahydrofuran (30 mL) was added dropwise. The mixture was allowed to warm to 5° C. and stirred at this temperature for 3 hours, then quenched with aqueous ammonium chloride solution. The mixture was extracted several times with ethyl acetate. The combined organic extracts were dried (MgSO₄), filtered and concentrated. Purification by flash chromatography afforded the title compound (0.121 g).

LCMS: C₃₁H₃₂FN₆O₄ requires 557.2; seen M/Z 558.3, [M+H]⁺, 556.4 [M−H]⁻. Retention time 3.37 min.

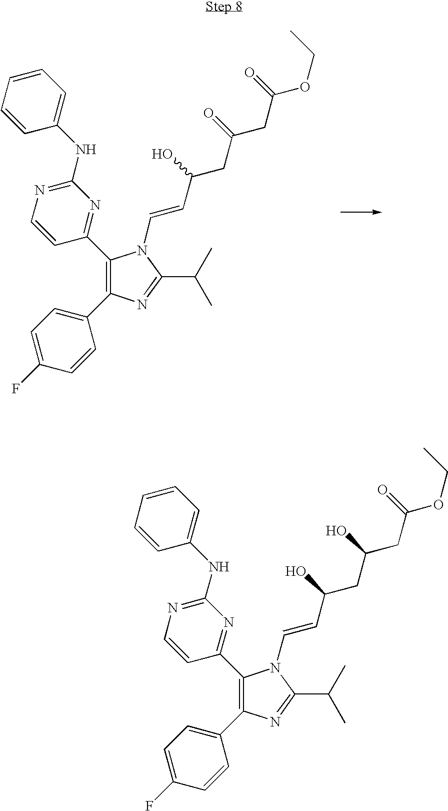

### (3R,5S,E) and (3S,5R,E)-ethyl 7-(4-(4-fluorophenyl)-2-isopropyl-5-(2-(phenylamino)pyrimidin-4-yl)-1H-imidazol-1-yl)-3,5-dihydroxyhept-6-enoate

A solution of (S and R,E)-ethyl 7-(4-(4-fluorophenyl)-2-isopropyl-5-(2-(phenylamino)pyrimidin-4-yl)-1H-imidazol-1-yl)-5-hydroxy-3-oxohept-6-enoate (0.115 g) in a mixture of tetrahydrofuran (2 mL) and methanol (0.2 mL) under nitrogen at −78° C. was treated with diethylmethoxyborane (0.021 g) dropwise. The mixture was left to stir at −78° C. for 45 minutes, then powdered sodium borohydride (0.008 g) was added in one portion. The mixture was again left to stir at −78° C. for 45 minutes, then quenched with aqueous ammonium chloride solution. On warming to room temperature, the mixture was extracted with dichloromethane, dried (MgSO₄), filtered and concentrated. The crude residue was purified by flash chromatography, then triturated with ether to afford the title compound (0.046 g).

LCMS: C₃₁H₃₄FN₅O₄ requires 559.3; seen M/Z 560.3, [M+H]⁺. Retention time 3.19 min.

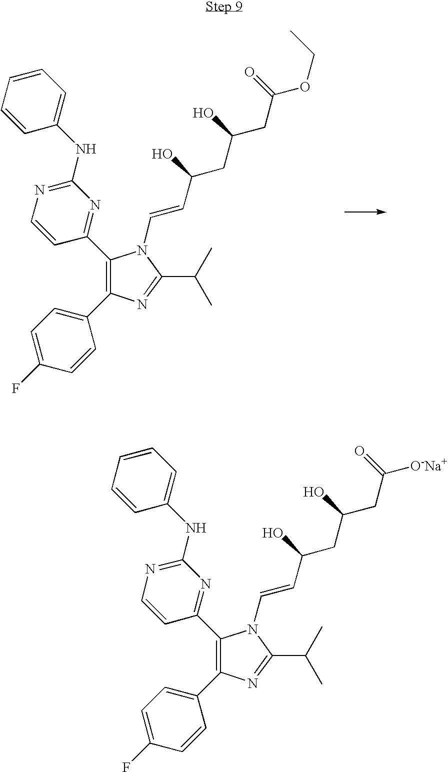

### (3R,5S,E) and (3S,5R,E)-7-(4-(4-fluorophenyl)-2-isopropyl-5-(2-(phenylamino)pyrimidin-4-yl)-1H-imidazol-1-yl)-3,5-dihydroxyhept-6-enoic acid sodium salt

A suspension of (3R,5S,E) and (3S,5R,E)-ethyl 7-(4-(4-fluorophenyl)-2-isopropyl-5-(2 (phenylamino)pyrimidin-4-yl)-1H-imidazol-1-yl)-3,5-dihydroxyhept-6-enoate (0.042 g) in ethanol (3 mL) was treated with aqueous sodium hydroxide (1M, 0.075 mL) and the mixture stirred at room temperature for 1 hour. The solvents were removed in vacuo to afford the title compound (0.040 g).

LCMS: C₂₉H₃₀FN₅O₄ requires 531.2; seen M/Z 532.3, [M+H]⁺, 530.4 [M−H]⁻. Retention time 1.70 min

¹H-NMR (400 MHz, DMSO-d₆, δ)—1.30 (d, 6H), 1.30-1.34 (m, 1H), 1.47-1.54 (m, 1H), 1.78 (dd, 1H), 1.99 (dd, 1H), 3.21 (sept, 1H), 3.61-3.70 (m, 1H), 5.19-5.27 (m, 1H), 5.65 (dd, 1H), 6.75 (d, 1H), 6.81 (d, 1H), 6.90 (t, 1H), 7.09-7.19 (m, 4H), 7.49-7.57 (5H), 8.45 (d, 1H), 9.69 (s, 1H).

### Example 29

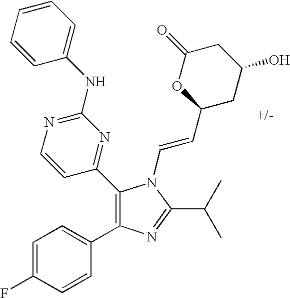

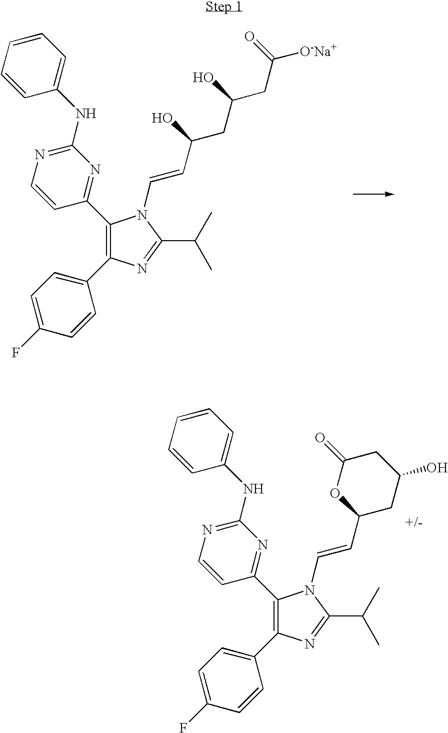

### (4R,6S) and (4S,6R)-6-((E)-2-(4-(4-fluorophenyl)-2-isopropyl-5-(2-(phenylamino)pyrimidin-4-yl)-1H-imidazol-1-yl)vinyl)-4-hydroxytetrahydro-2H-pyran-2-one

(3R,5S,E) and (3S,5R,E)-7-(4-(4-fluorophenyl)-2-isopropyl-5-(2-(phenylamino)pyrimidin-4-yl)-1H-imidazol-1-yl)-3,5-dihydroxyhept-6-enoic acid sodium salt (0.021 g) was dissolved in a mixture of 1,2-dichloroethane (0.75 mL) and acetonitrile (0.25 mL) and treated with trifluoroacetic acid (2 drops). The mixture was heated at 50° C. for 3 hours then diluted with dichloromethane and partitioned over aqueous sodium hydrogencarbonate solution. The organic layer was dried (MgSO₄), filtered and concentrated. Purification by flash chromatography afforded the title compound (0.012 g).

LCMS: C₂₉H₂₈FN₅O₃ requires 513.2; seen M/Z 514.3, [M+H]⁺, 512.4 [M−H]⁻. Retention time 3.04 min

¹H-NMR (400 MHz, CDCl₃, δ)—1.39 (d, 6H), 1.58-1.66 (m, 2H), 1.71-1.78 (m, 2H), 3.18 (sept, 1H), 4.18-4.22 (m, 1H), 5.16-5.22 (m, 1H), 5.59 (dd, 1H), 6.58 (d, 1H), 7.00-7.11 (m, 4H), 7.22 (s, 1H), 7.34 (t, 2H), 7.49-7.53 (m, 2H), 7.60 (d, 2H), 8.25 (d, 1H).

### Example 33

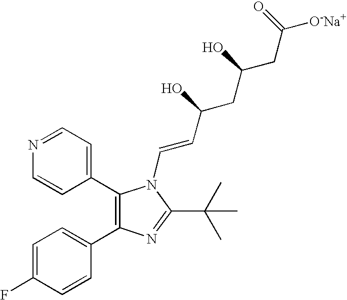

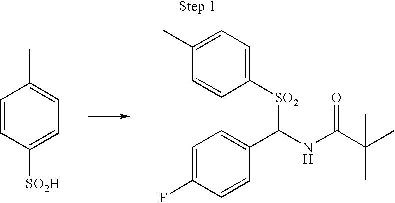

### N-((4-fluorophenyl)(tosyl)methyl)pivalamide

To a flask containing 4-fluorobenzaldehyde (41.3 g), trimethylacetamide (50.59 g), acetonitrile (200 mL) and toluene (200 mL) was added trimethylsilyl chloride (46.9 mL) and the resulting suspension stirred at 50° C. for 3 hours then cooled to room temperature. Para-toluene sulfinic acid (78 g) was added in one portion and the resulting suspension heated at 50° C. for a further 3 hours then at room temperature for 16 hours. The crude material was stirred over tert-butyl methyl ether (400 mL) and water (500 mL) for 10 minutes, then filtered washing with tert-butyl methyl ether, water and ether to furnish the desired compound (81 g).

¹H-NMR (400 MHz, CDCl₃, δ)—1.08 (s, 9H), 2.44 (s, 3H), 6.28 (d, 1H), 6.67 (d, 1H), 7.11 (t, 2H), 7.33 (d, 2H), 7.75 (d, 2H).

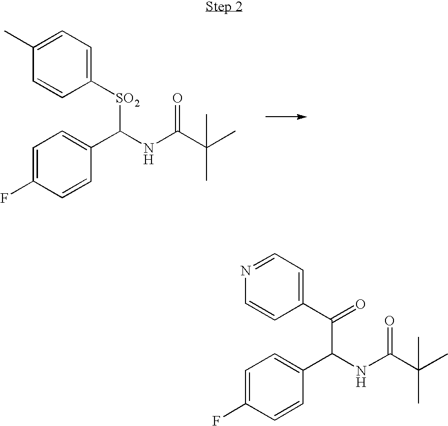

### N-(1-(4-fluorophenyl)-2-oxo-2-(pyridin-4-yl)ethyl)pivalamide

A mixture of N-((4-fluorophenyl)(tosyl)methyl)pivalamide (81 g) and 3-methyl-5-(2-hydroxyethyl)-4-methylthiazolium iodide (9.54 g) was treated with dichloromethane (1.2 L) and 4-pyridinecarboxaldehyde (26.29 g). The mixture was purged with nitrogen, then heated to 40° C. and triethylamine (470 mL) added in one portion. After heating at 40° C. for 6 hours, the mixture was cooled to room temperature and stirred for 16 hours then the solvent removed in vacuo. The crude residue was used without further purification in the next step.

LCMS: C₁₈H₁₉FN₂O₂ requires 314.1; seen M/Z 315.2 [M+H]⁺. Retention time 2.67 min.

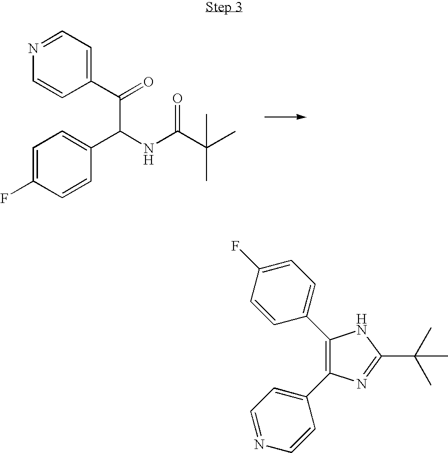

### 4-(2-tert-butyl-5-(4-fluorophenyl)-1H-imidazol-4-yl)pyridine

N-(1-(4-fluorophenyl)-2-oxo-2-(pyridin-4-yl)ethyl)pivalamide (70.0 g) was dissolved in ethanol (1 L) and ammonium acetate (258 g) added. The mixture was heated to reflux for 16 hours then cooled to room temperature. A copious quantity of water was added to precipitate the product which was filtered and washed with water. The crude residue was triturated with acetonitrile and refiltered, washing with acetonitrile and ether to afford the title compound (45 g).

LCMS: C₁₈H₁₈FN₃ requires 295.1; seen M/Z 296.2 [M+H]⁺. Retention time 2.70 min.

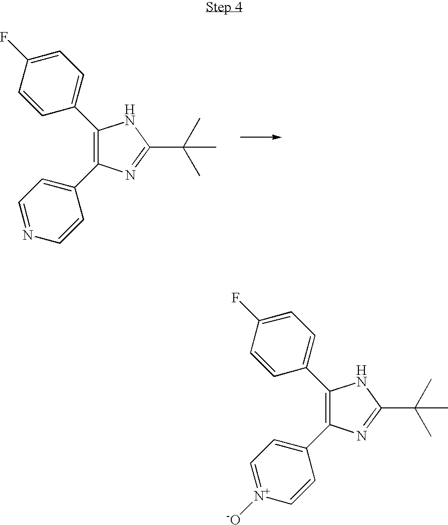

### 4-(2-tert-butyl-5-(4-fluorophenyl)-1H-imidazol-4-yl)pyridine 1-oxide

4-(2-tert-butyl-5-(4-fluorophenyl)-1H-imidazol-4-yl)pyridine (5.0 g) was dissolved in a mixture of dichloromethane (45 mL) and methanol (5 mL), purged with nitrogen and cooled to 0° C. Meta-chloroperoxybenzoic acid (7.89 g) was added portionwise over 10 minutes. The mixture was left to stir at 0° C. for 2 hours then diluted with dichloromethane and partitioned over water. The organic layer was washed with aqueous sodium metabisulfite and aqueous sodium hydrogencarbonate, dried (MgSO₄), filtered and concentrated. The residue was triturated with ethyl acetate to afford the title compound (2.1 g).

LCMS: C₁₈H₁₈FN₃O requires 311.1; seen M/Z 312.1 [M+H]⁺. Retention time 2.07 min.

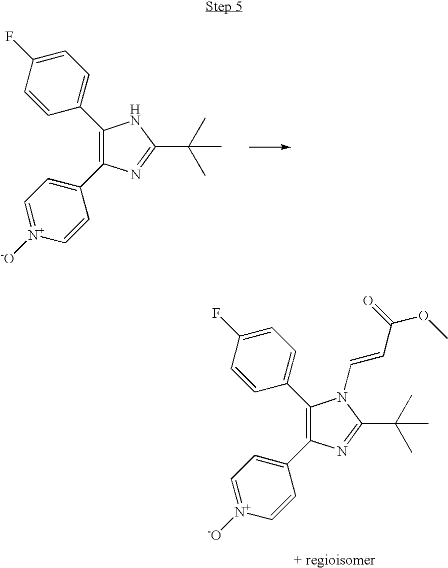

### (E)-4-(2-tert-butyl-5-(4-fluorophenyl)-1-(3-methoxy-3-oxoprop-1-enyl)-1H-imidazol-4-yl)pyridine 1-oxide

To a pre-dried flask was added 4-(2-tert-butyl-5-(4-fluorophenyl)-1H-imidazol-4-yl)pyridine 1-oxide (2.00 g) and tetrahydrofuran (70 mL) and the resultant slurry purged with nitrogen for 5 minutes. The flask was cooled to −10° C. and potassium hexamethyldisilazide (0.5M in toluene, 14.15 mL) was added dropwise over 10 minutes. The resultant mixture was stirred at −10° C. for 1 hour then methyl propiolate (7 mL) was added as a steady stream and the mixture left to warm to room temperature over 6 hours. Solvents were removed in vacuo and the residue filtered through celite with ethyl acetate. The solvent was again removed in vacuo and the residue purified by passing through a plug of silica to furnish a mixture of the two trans regioisomers (1.20 g).

LCMS: C₂₂H₂₂FN₃O₃ requires 395.2; seen M/Z 396.2 [M+H]⁺. Retention time 2.48 and 2.70 min.

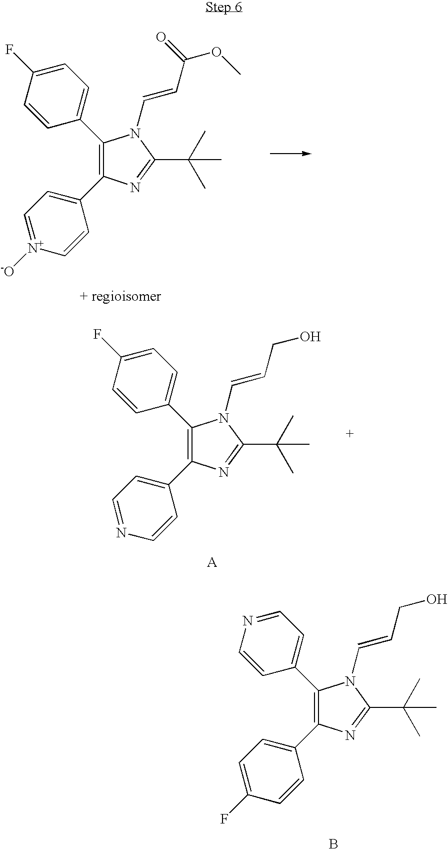

### (E)-3-(2-tert-butyl-5-(4-fluorophenyl)-4-(pyridin-4-yl)-1H-imidazol-1-yl)prop-2-en-1-ol A and (E)-3-(2-tert-butyl-4-(4-fluorophenyl)-5-(pyridin-4-yl)-1H-imidazol-1-yl)prop-2-en-1-ol B

To a pre-dried flask under nitrogen was added a solution of (E)-4-(2-tert-butyl-5-(4-fluorophenyl)-1-(3-methoxy-3-oxoprop-1-enyl)-1H-imidazol-4-yl)pyridine 1-oxide and (E)-5-(2-tert-butyl-4-(4-fluorophenyl)-1-(3-methoxy-3-oxoprop-1-enyl)-1H-imidazol-4-yl)pyridine 1-oxide (5.30 g) in dichloromethane (100 mL). Nitrogen was bubbled through the solution for 10 minutes then the mixture cooled to −78° C. and DIBAL-H (1M solution in toluene, 54 mL) added dropwise. Further DIBAL-H (1M solution in toluene, 30 mL) was added after stirring at −78° C. for 2 hours. The reaction mixture was left to warm to room temperature over 1 hour. After this time the reaction was quenched by addition of a mixture of sodium sulphate decahydrate and celite and left to stir for 30 minutes. The mixture was filtered, washing with copious ethyl acetate then concentrated. Purification by flash chromatography afforded (E)-3-(2-tert-butyl-5-(4-fluorophenyl)-4-(pyridin-4-yl)-1H-imidazol-1-yl)prop-2-en-1-ol (A, 0.87 g) and (E)-3-(2-tert-butyl-4-(4-fluorophenyl)-5-(pyridin-4-yl)-1H-imidazol-1-yl)prop-2-en-1-ol (B, 1.00 g).

A) LCMS: C₂₁H₂₂FN₃O requires 351.2; seen M/Z 352.2 [M+H]⁺. Retention time 2.73 min.

¹H-NMR (400 MHz, DMSO-d₆, δ)—1.42 (s, 9H), 3.85-3.90 (m, 2H), 4.97 (t, 1H), 5.55 (dt, 1H, J=14 Hz), 6.72 (dt, 1H, J=14 Hz), 7.24 (d, 2H), 7.27-7.35 (m, 2H), 7.34-7.39 (m, 2H), 8.34 (d, 2H).

B) LCMS: C₂₁H₂₂FN₃O requires 351.2; seen M/Z 352.2 [M+H]⁺. Retention time 2.77 min.

¹H-NMR (400 MHz, DMSO-d₆, δ)—1.41 (s, 9H), 3.86-3.92 (m, 2H), 4.96 (t, 1H), 5.49 (dt, 1H, J=14 Hz), 6.84 (dt, 1H, J=14 Hz), 7.04-7.10 (m, 2H), 7.26 (d, 2H), 7.31-7.36 (m, 2H), 8.57 (d, 2H).

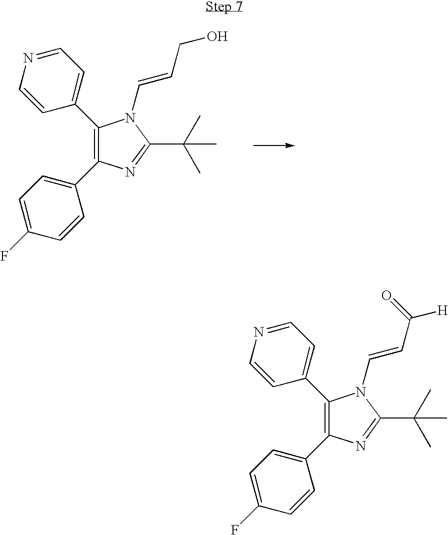

### (E)-3-(2-tert-butyl-4-(4-fluorophenyl)-5-(pyridin-4-yl)-1H-imidazol-1-yl)acrylaldehyde

A solution of (E)-3-(2-tert-butyl-4-(4-fluorophenyl)-5-(pyridin-4-yl)-1H-imidazol-1-yl)prop-2-en-1-ol B (1.00 g) in dichloromethane (45 mL) was treated with manganese dioxide (10.00 g) and the mixture stirred gently at room temperature for 24 hours. After filtration through celite, the reaction mixture was concentrated in vacuo to yield the desired compound (0.62 g) which was used without further purification

LCMS: C₂₁H₂₀FN₃O requires 349.2; seen M/Z 350.2 [M+H]⁺. Retention time 3.22 min.

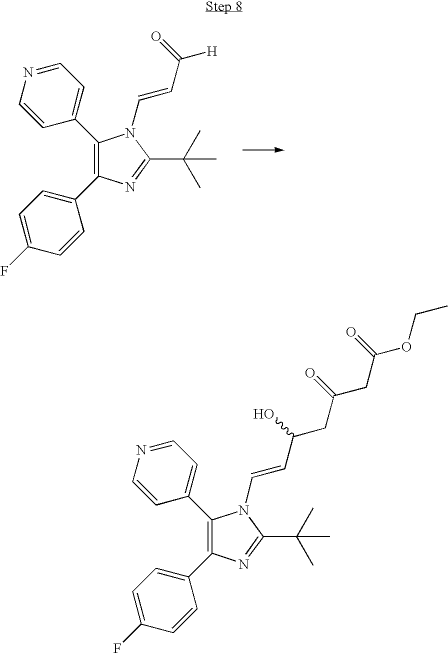

### (S and R,E)-ethyl 7-(2-tert-butyl-4-(4-fluorophenyl)-5-(pyridin-4-yl)-1H-imidazol-1-yl)-5-hydroxy-3-oxohept-6-enoate

A flask containing sodium hydride (5.20 g) and tetrahydrofuran (70 mL) was cooled to −10° C. under nitrogen. Ethyl acetoacetate (1.69 g) in tetrahydrofuran (10 mL) was added dropwise and the mixture stirred at −10° C. for 10 minutes. Butyllithium (1.6 M in hexanes, 12.2 mL) was then added dropwise. The flask was allowed to warm to 0° C. over 30 minutes then re-cooled to −10° C. whereupon a solution of (E)-3-(2-tert-butyl-4-(4-fluorophenyl)-5-(pyridin-4-yl)-1H-imidazol-1-yl)acrylaldehyde (2.27 g) in tetrahydrofuran (150 mL) was added dropwise. The mixture was allowed to warm to room temperature and stirred for 3 hours, then quenched with aqueous ammonium chloride solution. The mixture was extracted several times with dichloromethane. The combined organic extracts were dried (MgSO₄), filtered and concentrated. Excess mineral oil was removed by washing with iso-hexanes and the residue triturated with a mixture of ether and iso-hexanes to afford the desired compound (2.21 g).

LCMS: C₂₇H₃₀FN₃O₄ requires 479.2; seen M/Z 480.2 [M+H]⁺. Retention time 2.95 min.

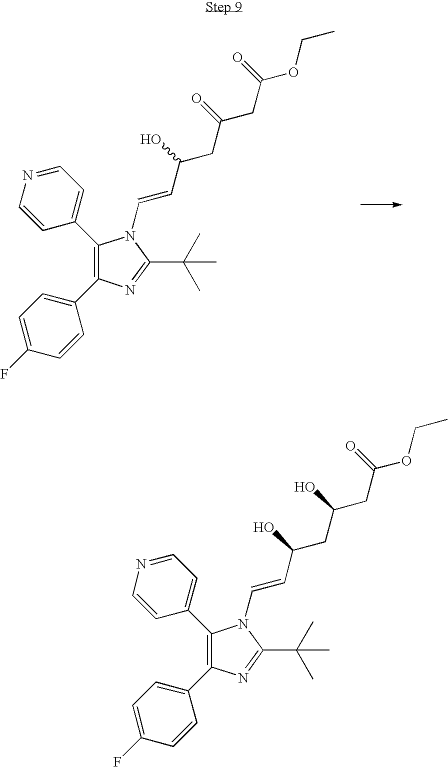

### (3R,5S,E) and (3S,5R,E)-ethyl 7-(2-tert-butyl-4-(4-fluorophenyl)-5-(pyridin-4-yl)-1H-imidazol-1-yl)-3,5-dihydroxyhept-6-enoate

A solution of (S and R,E)-ethyl 7-(2-tert-butyl-4-(4-fluorophenyl)-5-(pyridin-4-yl)-1H-imidazol-1-yl)-5-hydroxy-3-oxohept-6-enoate (1.78 g) in a mixture of tetrahydrofuran (27 mL) and methanol (9 mL) under nitrogen at −78° C. was treated with diethylmethoxyborane (0.38 mL) dropwise. The mixture was left to stir at −78° C. for 45 minutes, then powdered sodium borohydride (0.14 g) added in one portion. The mixture was again left to stir at −78° C. for 45 minutes, then quenched with aqueous ammonium chloride solution. On warming to room temperature, the mixture was extracted with dichloromethane, dried (MgSO₄), filtered and concentrated. The crude residue was purified by flash chromatography to afford the target compound (1.11 g).

LCMS: C₂₇H₃₂FN₃O₄ requires 481.2; seen M/Z 482.3 [M+H]⁺. Retention time 2.85 min.

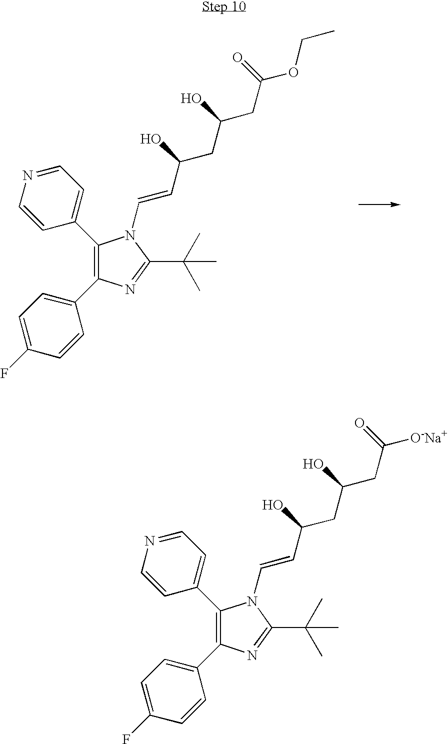

### (3R,5S,E) and (3S,5R,E)-7-(2-tert-butyl-4-(4-fluorophenyl)-5-(pyridin-4-yl)-1H-imidazol-1-yl)-3,5-dihydroxyhept-6-enoic acid sodium salt

A solution of (3R,5S,E) and (3S,5R,E)-ethyl 7-(2-tert-butyl-4-(4-fluorophenyl)-5-(pyridin-4-yl)-1H-imidazol-1-yl)-3,5-dihydroxyhept-6-enoate (1.11 g) in ethanol (40 mL) was treated with aqueous sodium hydroxide (1M, 2.30 mL) and the mixture stirred at room temperature for 1 hour. The solvents were removed in vacuo to afford the title compound (1.10 g).

LCMS: C₂₅H₂₈FN₃O₄ requires 453.2; seen M/Z 454.3 [M+H]⁺. Retention time 1.52 min.

¹H-NMR (400 MHz, DMSO-d₆, δ)—0.99-1.05 (m, 1H), 1.29-1.37 (m, 1H), 1.42 (s, 9H), 1.73 (dd, 1H), 1.94 (dd, 1H), 3.43-3.48 (m, 1H), 4.14-4.18 (m, 1H), 5.40 (dd, 1H), 6.79 (d, 1H), 7.08 (t, 2H), 7.26 (d, 2H), 7.32-7.37 (m, 2H), 8.57 (d, 2H).

### Example 34

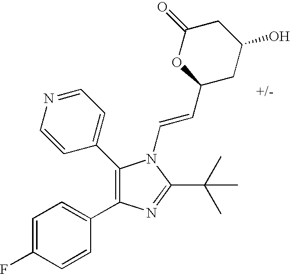

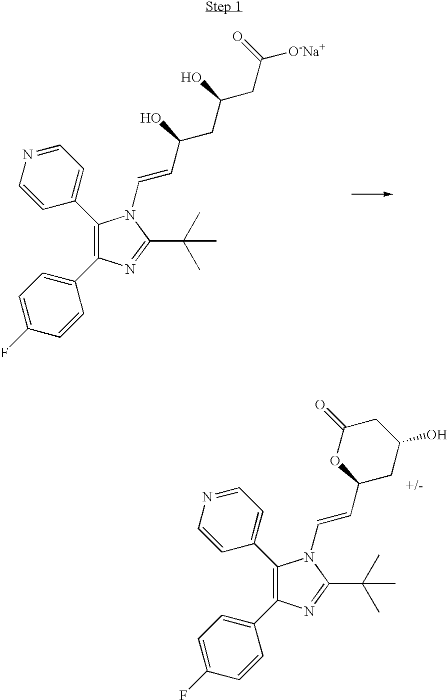

### (4R,6S) and (4S,6R)-6-((E)-2-(2-tert-butyl-4-(4-fluorophenyl)-5-(pyridin-4-yl)-1H-imidazol-1-yl)vinyl)-4-hydroxytetrahydro-2H-pyran-2-one

(3R,5S,E) and (3S,5R,E)-7-(2-tert-butyl-4-(4-fluorophenyl)-5-(pyridin-4-yl)-1H-imidazol-1-yl)-3,5-dihydroxyhept-6-enoic acid sodium salt (0.116 g) was dissolved in a mixture of 1,2-dichloroethane (4 mL) and acetonitrile (1 mL) and treated with trifluoroacetic acid (0.057 mL). The mixture was heated at 50° C. for 3 hours, then further trifluoroacetic acid (0.040 mL) added and the mixture left to stir at room temperature overnight. The mixture was then diluted with dichloromethane and partitioned over aqueous sodium hydrogencarbonate solution. The organic layer was dried (MgSO₄), filtered and concentrated. Purification by flash chromatography afforded the title compound (0.020 g).

LCMS: C₂₅H₂₆FN₃O₃ requires 435.2; seen M/Z 436.2 [M+H]⁺. Retention time 2.70 min.

¹H-NMR (400 MHz, DMSO-d₆, δ)—1.39-1.54 (m, 2H), 1.43 (s, 9H), 2.33-2.39 (m, 1H), 2.61 (dd, 1H), 4.00-4.04 (m, 1H), 5.13-5.20 (m, 1H), 5.27 (d, 1H), 5.48 (dd, 1H), 7.07-7.14 (m, 3H), 7.30 (d, 2H), 7.34-7.39 (m, 2H), 8.61 (d, 2H).

### Example 39

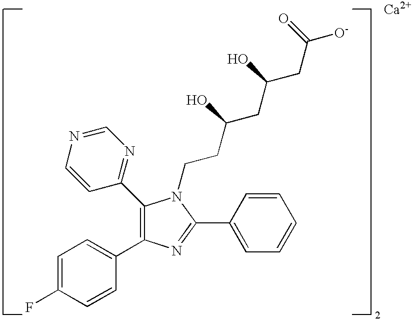

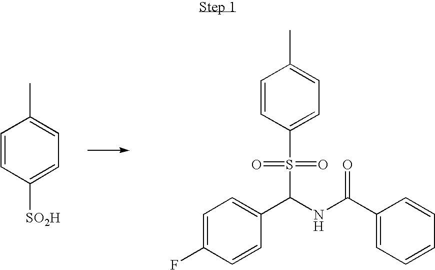

### N-((4-fluorophenyl)(tosyl)methyl)benzamide

A flask was charged with benzamide (10.0 g), acetonitrile (20 mL), toluene (20 mL), trimethylsilyl chloride (6.3 mL) and 4-fluorobenzaldehyde (5.0 g). The mixture was heated to 50° C. with stirring for 4 hours. Powdered para-toluene sulfinic acid (9.6 g) was added portionwise and the resulting mixture heated at 45° C. for 16 hours. On cooling, the mixture was partitioned between water and tert-butyl methyl ether, stirred for 10 minutes then filtered to afford the title compound which was dried under vacuum (10.1 g).

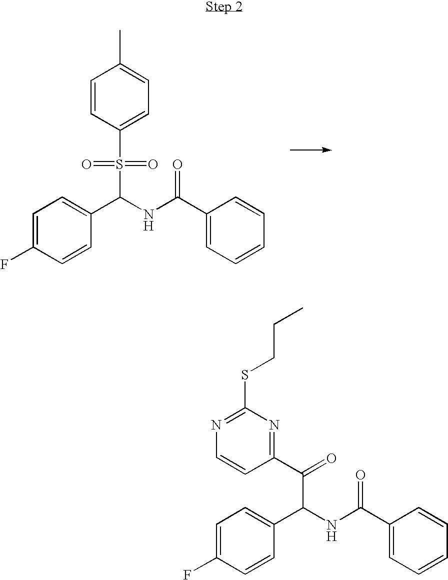

### N-(1-(4-fluorophenyl)-2-oxo-2-(2-(propylthio)pyrimidin-4-yl)ethyl)benzamide

A mixture of N-((4-fluorophenyl)(tosyl)methyl)benzamide (6.18 g) and 3-methyl-5-(2-hydroxyethyl)-4-methylthiazolium iodide (0.68 g) was treated with dichloromethane (70 mL) and 2-(propylthio)pyrimidine-4-carboxaldehyde (3.4 g). The mixture was purged with nitrogen, then heated to 40° C. and triethylamine (33 mL) added in one portion. After heating at 40° C. for 2 hours, the mixture was cooled to room temperature and the mixture poured into aqueous sodium hydrogencarbonate. The aqueous layer was extracted with dichloromethane three times and the combined organics dried (MgSO₄), filtered and concentrated. The crude residue was used without further purification in the next step (6.10 g).

LCMS: C₂₂H₂₀FN₃O₂S requires 409.1; seen M/Z 410.0, [M+H]⁺, 408.2 [M−H]⁻. Retention time 5.35 min.

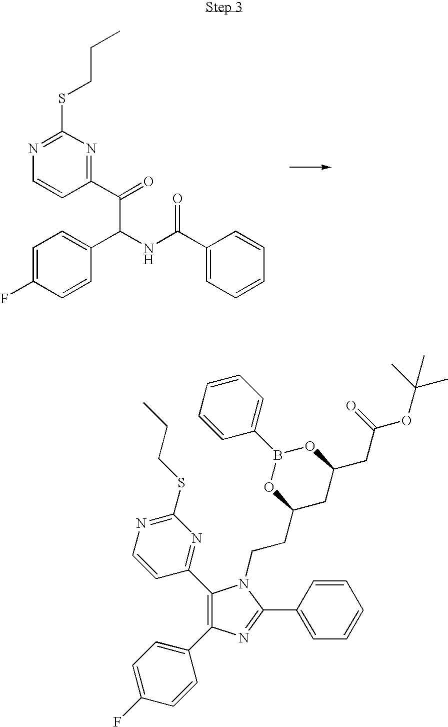

### tert-Butyl 2-((4R,6R)-6-(2-(4-(4-fluorophenyl)-2-phenyl-5-(2-(propylthio)pyrimidin-4-yl)-1H-imidazol-1-yl)ethyl)-2-phenyl-1,3,2-dioxaborinan-4-yl)acetate

A mixture of N-(1-(4-fluorophenyl)-2-oxo-2-(2-(propylthio)pyrimidin-4-yl)ethyl)benzamide (2.00 g), tert-butyl 2-((4R,6R)-6-(2-aminoethyl)-2-phenyl-1,3,2-dioxaborinan-4-yl)acetate, (4.70 g), acetic acid (0.85 mL) and ethanol (20 mL) were heated together for 16 hours at 90° C. The mixture was then partitioned between ethyl acetate and aqueous sodium hydrogencarbonate. The aqueous layer was extracted with further ethyl acetate and the combined organics dried (MgSO₄), filtered and concentrated. The crude residue was purified by flash chromatography to afford the title compound (2.80 g).

LCMS: C₃₉H₄₂BFN₄O₄S requires 692.3; seen M/Z 607.2 [M-C₆H₃B+H]⁺. Retention time 5.70 min.

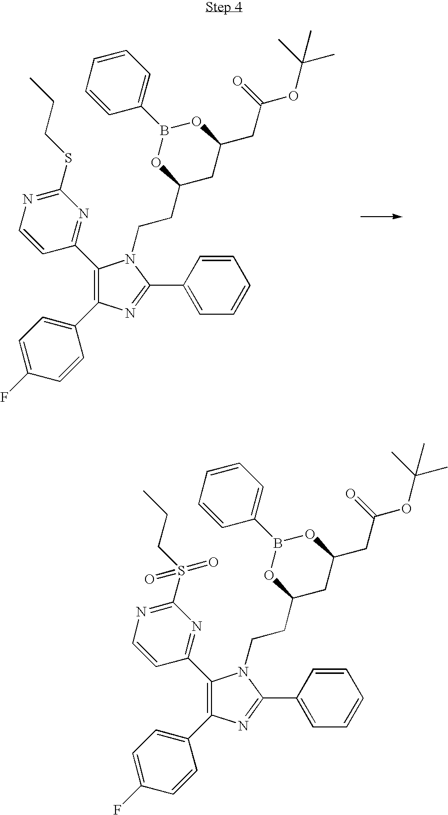

### tert-butyl 2-((4R,6R)-6-(2-(4-(4-fluorophenyl)-2-phenyl-5-(2-(propylsulfonyl)pyrimidin-4-yl)-1H-imidazol-1-yl)ethyl)-2-phenyl-1,3,2-dioxaborinan-4-yl)acetate

To solution of tert-butyl 2-((4R,6R)-6-(2-(4-(4-fluorophenyl)-2-phenyl-5-(2-(propylthio)pyrimidin-4-yl)-1H-imidazol-1-yl)ethyl)-2-phenyl-1,3,2-dioxaborinan-4-yl)acetate (1.10 g) in methanol (26 mL) was added a solution of oxone (2.93 g) in water (26 mL). The resulting suspension was stirred at room temperature for 2 hours. The methanol was removed in vacuo and the remaining aqueous portion treated with aqueous sodium hydrogencarbonate solution, then extracted with ethyl acetate. The organic layer was washed with brine, dried (MgSO₄), filtered and concentrated to afford the title compound (0.90 g) which was used without further purification in the next step.

LCMS: C₃₉H₄₂BFN₄O₆S requires 724.3; seen M/Z 639.2 [M-C₆H₃B+H]⁺, 637.3 [M-C₆H₃B—H]⁻. Retention time 4.92 min.

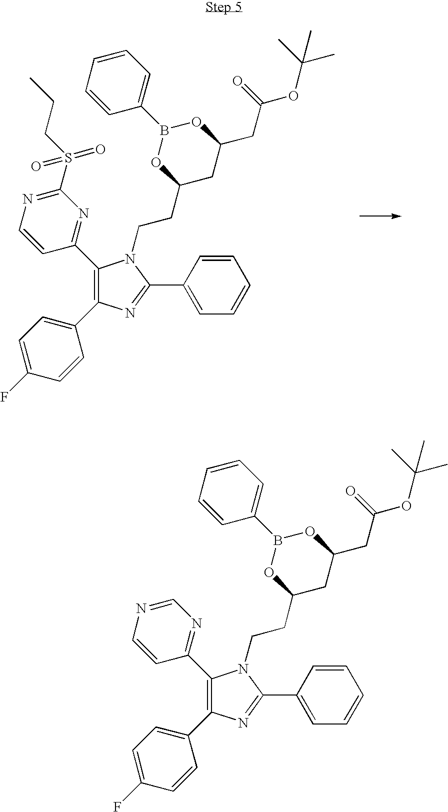

### tert-butyl 2-((4R,6R)-6-(2-(4-(4-fluorophenyl)-2-phenyl-5-(pyrimidin-4-yl)-1H-imidazol-1-yl)ethyl)-2-phenyl-1,3,2-dioxaborinan-4-yl)acetate

To a solution of tert-butyl 2-((4R,6R)-6-(2-(4-(4-fluorophenyl)-2-phenyl-5-(2-(propylsulfonyl)pyrimidin-4-yl)-1H-imidazol-1-yl)ethyl)-2-phenyl-1,3,2-dioxaborinan-4-yl)acetate (0.90 g) in ethanol (9 mL) under nitrogen was added sodium borohydride (0.047 g) and the mixture stirred at room temperature for 16 hours. A solution of hydrochloric acid (2M) was added until the mixture turned acidic, then aqueous sodium hydrogencarbonate was added to neutralise. The ethanol was removed in vacuo and the remaining aqueous portion extracted with dichloromethane, washed with brine, dried (MgSO₄), filtered and concentrated. The title compound was isolated by purification by flash chromatography (0.27 g).

LCMS: C₃₆H₃₆BFN₄O₄ requires 618.3; seen M/Z 533.2 [M-C₆H₃B+H]⁺. Retention time 4.45 min.

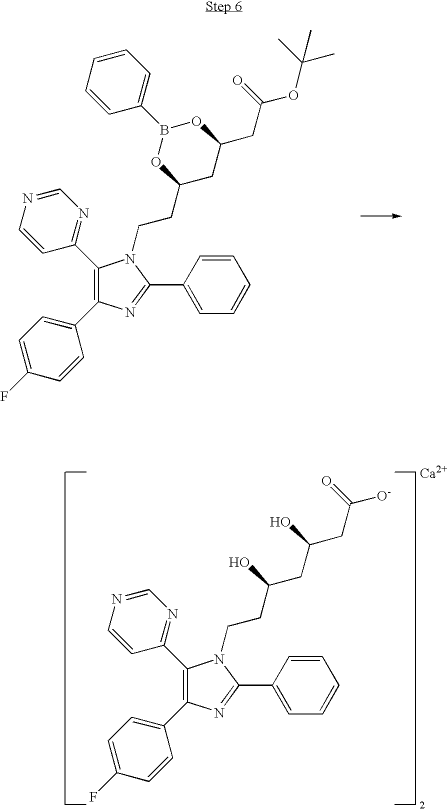

### (3R,5R)-7-(4-(4-fluorophenyl)-2-phenyl-5-(pyrimidin-4-yl)-1H-imidazol-1-yl)-3,5-dihydroxyheptanoic acid calcium salt

To a stirred solution of aqueous sodium hydroxide (1M, 0.77 mL) was added aqueous hydrogen peroxide (35% wt, 0.076 mL). The mixture was left to stir at room temperature for 30 minutes. To this was added a solution of tert-butyl 2-((4R,6R)-6-(2-(4-(4-fluorophenyl)-2-phenyl-5-(pyrimidin-4-yl)-1H-imidazol-1-yl)ethyl)-2-phenyl-1,3,2-dioxaborinan-4-yl)acetate (0.24 g) in tetrahydrofuran (2 mL) and the mixture was left to stir at room temperature for 6 hours. The reaction mixture was partitioned between ethyl acetate and water and the aqueous layer washed twice ethyl acetate, then neutralised with hydrochloric acid (1M). To the aqueous layer was added aqueous calcium chloride (0.118M, 0.50 mL) precipitating the title compound (0.031 g) which was filtered, washing with water and acetonitrile.

LCMS: C₂₆H₂₅FN₄O₄ requires 476.2; seen M/Z 477.0 [M+H]⁺, 475.2 [M−H]⁻. Retention time 2.05 min.

¹H-NMR (400 MHz, DMSO-d₆, δ)—1.05-1.17 (m, 1H), 1.21-1.30 (m, 1H), 1.36-1.55 (m, 2H), 1.87 (dd, 1H), 2.00 (dd, 1H), 3.39-3.47 (m, 1H), 3.58-3.67 (m, 1H), 4.23-4.48 (m, 2H), 7.18 (t, 2H), 7.42-7.48 (m, 3H), 7.50-7.57 (m, 3H), 7.73 (d, 2H), 8.78 (d, 1H), 9.33 (s, 1H).

### Example 40

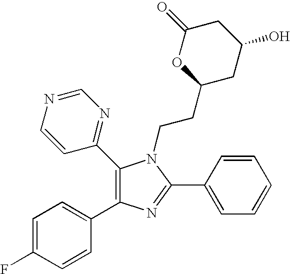

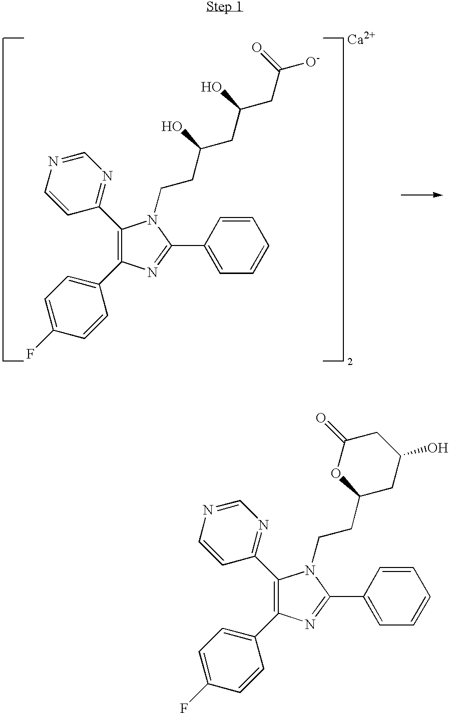

### (4R,6R)-6-(2-(4-(4-fluorophenyl)-2-phenyl-5-(pyrimidin-4-yl)-1H-imidazol-1-yl)ethyl)-4-hydroxytetrahydro-2H-pyran-2-one

(3R,5R)-7-(4-(4-fluorophenyl)-2-phenyl-5-(pyrimidin-4-yl)-1H-imidazol-1-yl)-3,5-dihydroxyheptanoic acid calcium salt (0.067 g) was dissolved in acetonitrile (1 mL) and trifluoroacetic acid (0.011 mL) was added. The mixture was heated to 70° C. for 4 hours then stirred at room temperature for 16 hours and poured into aqueous sodium hydrogencarbonate solution. After neutralising with hydrochloric acid (1M), the aqueous layer was extracted with ethyl acetate, dried (MgSO₄), filtered and concentrated. Purification by flash chromatography afforded the title compound (0.008 g).

LCMS: C₂₆H₂₃FN₄O₃ requires 458.2; seen M/Z 459.1 [M+H]⁺. Retention time 3.84 min.

¹H-NMR (270 MHz, CDCl₃, δ)—1.44-2.04 (m, 4H), 2.42-2.68 (m, 2H), 4.23-4.32 (m, 1H), 4.46-4.73 (m, 3H), 7.03 (d, 2H), 7.19-7.26 (m, 1H), 7.44-7.53 (m, 5H), 7.63-7.66 (m, 2H), 8.55 (d, 1H), 9.28 (s, 1H).

### Example 41

The biological activities of compounds of the present invention were measured in assays which include:
1) Inhibition of rat liver HMG-CoA reductase. Further details on the assay are present in Heller, R. A.; Gould, R. G. Biochem. Biophys. Research Commun. 1973, 50, 859 and Kubo, M.; Strott, C. A.; Endocrinology 1987, 120, 214.
2) Inhibition of recombinant human p38αMAPK
3) Inhibition of lipopolysaccharide (LPS)-stimulated release of tumor necrosis factor alpha (TNFα) from human peripheral blood mononuclear cells (PBMC).

Results from these measurements are presented in Table 1, below.

TABLE 1

| | HMG-CoA R | p38α MAPK | TNFα Release |
|---|---|---|---|
| Compound | IC₅₀ (μM)^{a} | IC₅₀ (μM)^{b} | IC₅₀ (μM)^{d} |
| Example 24 | NT^{d} | 0.11 | <3.0 |
| Example 25 | 0.098 | <0.10 | 9.7 |
| Example 28 | 0.0048 | 0.27 | 9.7 |
| Example 29 | NT | <0.10 | 19 |
| Example 33 | 0.011 | 6.0 | 230 |
| Example 34 | NT | 1.8 | 190 |
| Example 39 | >1.0 | 14 | >300 |
| Example 40 | NT | 1.4 | 26 |

^{a}Concentration of compound required to inhibit rat liver hydroxymethylglutaryl-CoA reductase activity by 50%.
^{b}Concentration of compound required to inhibit recombinant human p38α MAPK activity by 50%.
^{c}Concentration of compound required to inhibit lipopolysaccharide-stimulated release of tumor necrosis factor alpha (TNFα) from human peripheral blood mononuclear cells (PBMC) by 50%.
^{d}NT = Not tested.

Human p38αMAP Kinase Inhibition Assay: In vitro cell-free p38αMAP kinase inhibition assays were conducted by the method as described in Clerk et al., FEBS Lett., 426:93-96 (1998) for a number of lactones in formulas I/IIa/IIb. Briefly, human recombinant p38α protein kinase expressed in E. coli (UBI #14-251) was used. Myelin basic protein (MBP, UBI #13-110) was employed as substrate, and microtiter plate wells were coated with MBP (0.01 mg/ml) overnight at 4° C. Candidate compound and/or vehicle was preincubated with 0.075 μg/mL enzyme in modified HEPES buffer pH 7.4 at 25° C. for 15 minutes. The reaction was initiated by addition of 100 μM ATP and allowed to proceed for another 60 minutes. The reaction was terminated by aspirating the solution. Phosphorylated MBP was detected by incubation with a mouse monoclonal IgG2a anti-phosphoMBP antibody. Bound anti-phosphoMBP antibody was quantitated by incubation with a HRP conjugated goat anti-mouse IgG. The protein kinase activity was proportional to the readings of optical density at 405 nm resulting from reaction with an ABTS Microwell Peroxidase Substrate System.

Using this method, IC₅₀ data was obtained, with results illustrated in Table 1, as discussed above.

Whole Cell LPS-Stimulated TNF-α Release Assay: The procedure as described in Welker et al., Int. Arch. Allergy and Immunol. 109:110-115 (1996) was followed. Briefly, a candidate compound and/or vehicle was preincubated with human peripheral blood mononuclear leukocytes (PBML, 5×10⁵/ml) cells in AIM-V medium pH 7.4 for 2 hours. Lipopolysaccharide (LPS, 25 ng/ml) was added to stimulate the cells, which was incubated overnight at 37° C. TNF-α cytokine levels in the conditioned medium was then quantitated using a sandwich ELISA kit.

1. A method of inhibiting a MAP kinase comprising administering an effective amount of at least one compound comprising of formula XXXII: 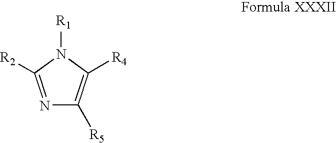
wherein R₁ is 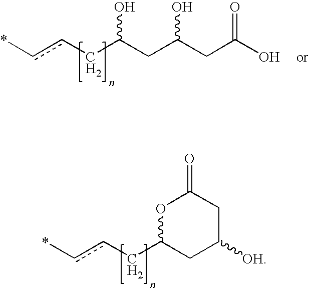
n being 0 or any integer;
R₂ is optionally substituted alkyl, cycloalkyl, aryl, or heteroaryl;
R₄ is optionally substituted 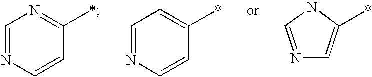
and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a pharmaceutically acceptable salt thereof.

2. The method as recited in claim 1 wherein said R₁ has the following stereochemistry:

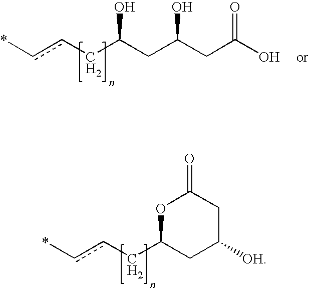

3. The method as recited in claim 1 wherein said inhibited MAP kinase is p38 MAP kinase.

4. The method as recited in claim 1, further comprising inhibiting an HMG CoA reductase.

5. The method as recited in claim 1 wherein said administering treats a MAP kinase-related condition.

6. The method as recited in claim 1 wherein said administering treats a MAP kinase-related condition and an HMG CoA reductase-related condition.

7. The method as recited in claim 1 wherein said administering treats an inflammatory condition.

8. A compound of formula XXXII: 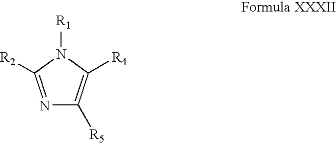
wherein R₁ is 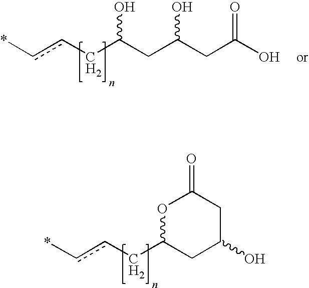
n being 0 or any integer;
R₂ is optionally substituted alkyl, cycloalkyl, aryl, or heteroaryl;
R₄ is optionally substituted 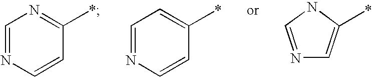
and R₅ is hydrogen, optionally substituted lower alkyl, optionally substituted cycloalkyl, optionally substituted aryl, optionally substituted heteroaryl, halogen, cyano, hydroxy, optionally substituted lower alkoxy, optionally substituted alkaryl, optionally substituted alkheteroaryl, optionally substituted amido, optionally substituted carboxamido, optionally substituted sulfonamide, optionally substituted amidosulfonyl, or optionally substituted amino, or a pharmaceutically acceptable salt thereof.

9. A compound comprising at least one structure selected from: 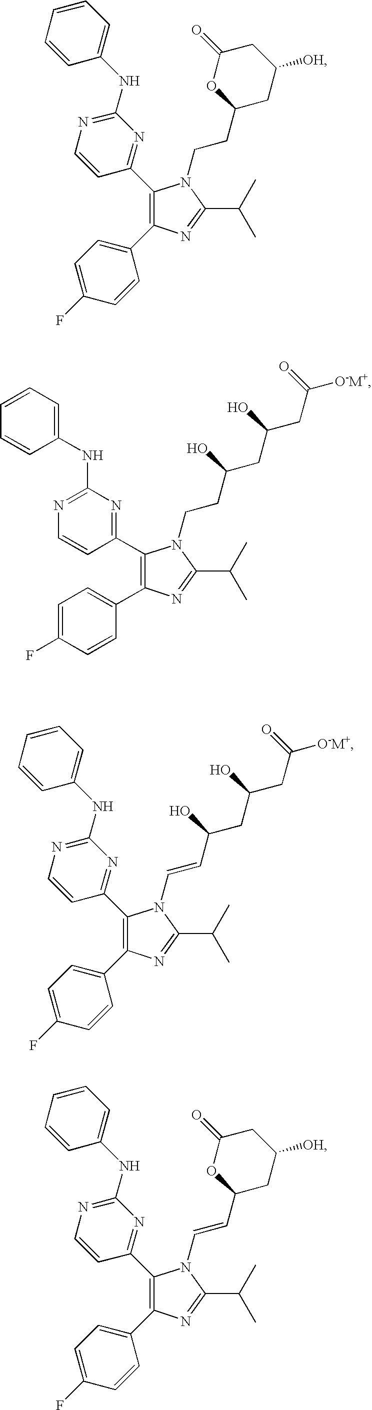 [chemical structure]
